SEQUENCE LISTING (13 sequences)

SEQ ID NO: 1 - protein, 467 aa, Artificial Sequence
  Antibody sequence
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 240 aa, Artificial Sequence
  Antibody sequence
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 474 aa, Artificial Sequence
  Antibody sequence
MRVMAPRTLILLLSGALALTETWAGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 240 aa, Artificial Sequence
  Antibody sequence
MRVMAPRTLILLLSGALALTETWAGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 5 - protein, 8 aa, Artificial Sequence
  CDR
GYTFTSYW

SEQ ID NO: 6 - protein, 8 aa, Artificial Sequence
  CDR
IYPSDSYT

SEQ ID NO: 7 - protein, 11 aa, Artificial Sequence
  CDR
TRSWRGNSFDY

SEQ ID NO: 8 - protein, 12 aa, Artificial Sequence
  CDR
QSLLNSGNQKNY

SEQ ID NO: 9 - protein, 3 aa, Artificial Sequence
  CDR
WAS

SEQ ID NO: 10 - protein, 9 aa, Artificial Sequence
  CDR
QNDYSYPFT

SEQ ID NO: 11 - protein, 19 aa, Artificial Sequence
  Secretion signal
MGWSCIILFLVATATGVHS

SEQ ID NO: 12 - protein, 20 aa, Artificial Sequence
  Secretion signal
MESQTQVLMSLLFWVSGTCG

SEQ ID NO: 13 - protein, 26 aa, Artificial Sequence
  CDR
MRVMAPRTLILLLSGALALTETWAGS

(19) United States
(12) Patent Application Publication
(10) Pub. No.: US 2024/0043527 A1
(43) Pub. Date: Feb. 8, 2024

(54) RNA COMPOSITIONS TARGETING CLAUDIN-18.2

(71) Applicant: BioNTech SE, Mainz (DE)

(72) Inventors: Ugur Sahin, Mainz (DE); Claudia Lindemann, Mainz (DE); Jan Diekmann, Mainz (DE); Kerstin Brettschneider, Mainz (DE); Hayat Bähr-Mahmud, Mainz (DE); Ursula Ellinghaus, Mainz (DE); Leyla Fischer, Mainz (DE); Christiane Stadler, Mainz (DE); Özlem Türeci, Mainz (DE)

(21) Appl. No.: 17/915,567

(22) PCT Filed: Mar. 29, 2021
(86) PCT No.: PCT/EP21/58112

§ 371 Date: Sep. 29, 2022

Related U.S. Application Data
(60) Provisional application No. 63/002,287, filed on Mar. 30, 2020.

(51) Int. Cl. C07K 16/28; A61P 35/00

(52) U.S. Cl. CPC C07K 16/28; A61P 35/00; C07K 2317/522; C07K 2317/14

(57) ABSTRACT

The present disclosure provides RNA technologies for targeting Claudin-18.2 polypeptides. In some embodiments, such RNA technologies can be useful for treatment of diseases associated with positive expression of Claudin-18.2. For example, in some embodiments, such RNA technologies can be useful for treatment of Claudin-18.2 positive cancer, including, e.g, but not limited to biliary cancers, ovarian cancers, gastric cancers, gastro-esophageal cancers, pancreatic cancers. In some embodiments, such RNA technologies can be used in combination therapy (e.g, in combination with a chemotherapeutic agent).

### BACKGROUND

Cancer is the second leading cause of death globally and is expected to be responsible for an estimated 9.6 million deaths in 2018 (Bray et al. 2018). In general, once a solid tumor has metastasized, with a few exceptions such as germ cell and some carcinoid tumors, 5-year survival rarely exceeds 25%.

Conventional therapies such as chemotherapy, radiotherapy, surgery, and targeted therapies and recent advances in immunotherapies have improved outcomes in patients with advanced solid tumors. In the last few years, the Food and Drug Administration (FDA) and European Medicines Agency (EMA) have approved eight checkpoint inhibitors (one monoclonal antibody targeting the CTLA-4 pathway, ipilimumab, and seven antibodies targeting programmed death receptor/ligand [PD/PD-L1], including atezolizumab, avelumab, durvalumab, nivolumab, cemiplimab and pembrolizumab), for the treatment of patients with multiple cancer types, mainly solid tumors. These approvals have dramatically changed the landscape of cancer treatment. However, certain cancers such as pancreatic adenocarcinoma or metastatic biliary tract cancers still do not yet benefit from existing therapies including immunotherapies.

### SUMMARY

The poor prognosis of certain cancers such as, e.g., pancreatic and biliary cancer types, highlights the need for additional treatment approaches. The present disclosure, among other things, provides an insight that Claudin-18.2 (CLDN-18.2) represents a particularly useful tumor-associated antigen against which therapies may be targeted. Without wishing to be bound by any particular theory, the present disclosure notes that CLDN-18.2's tissue expression pattern, including its particularly limited expression in non-cancer tissues, may contribute to its usefulness as a target as described herein. To date, no therapy targeting CLDN-18.2 has been approved for any cancer indication.

The present disclosure further provides an insight that, in some embodiments, therapy targeting CLDN-18.2, as described herein, may usefully involve administration of an antibody agent that targets CLDN-18.2. Moreover, the present disclosure provides a particular insight that a particularly beneficial strategy for delivering such an antibody agent may be by administration of a nucleic acid encoding the antibody agent. Still further, the present disclosure provides a particular insight that delivery of RNA (e.g., ssRNA such as mRNA encoding the antibody agent) via lipid nanoparticles targeting liver cells may be a particularly beneficial strategy for delivering such an antibody agent.

Those skilled in the art will be aware of the burgeoning field of nucleic acid therapeutics, and moreover of RNA (e.g., ssRNA such as mRNA) therapeutics (see, for example, mRNA-encoding proteins and/or cytokines). Various embodiments of technologies provided herein may utilize particular features of developed RNA (e.g., ssRNA such as mRNA) therapeutic technologies and/or delivery systems. For example, in some embodiments, an administered RNA (e.g., ssRNA such as mRNA) may comprise one or more modified nucleotides (e.g., but not limited to pseudouridine), nucleosides, and/or linkages. Alternatively or additionally, in some embodiments, an administered RNA (e.g., ssRNA such as mRNA) may comprise a modified polyA sequence (e.g., a disrupted polyA sequence) that enhances stability and/or translation efficiency. Alternatively or additionally, in some embodiments, an administered RNA (e.g., ssRNA such as mRNA) may comprise a specific combination of at least two 3′UTR sequences (e.g., a combination of a sequence element of an amino terminal enhancer of split RNA and a sequence derived from a mitochondrially encoded 12S RNA). Alternatively or additionally, in some embodiments, an administered RNA (e.g., ssRNA such as mRNA) may comprise a ‘5 UTR sequence that is derived from human α-globin mRNA. Alternatively or additionally, in some embodiments, an administered RNA (e.g., ssRNA such as mRNA) may comprise a 5’ cap analog, e.g., for co-transcriptionally capping. Alternatively or additionally, in some embodiments, an administered RNA (e.g., ssRNA such as mRNA) may comprise a secretion signal-coding region with reduced immunogenicity (e.g., a human secretion signal-coding sequence) such that an encoded antibody agent is expressed and secreted. In some embodiments, an administered RNA may be formulated in or with one or more delivery vehicles (e.g., nanoparticles such as lipid nanoparticles, etc.). Alternatively or additionally, in some embodiments, an administered RNA may be formulated in or with liver-targeting lipid nanoparticles (e.g., cationic lipid nanoparticles).

The present disclosure further provides an insight that a RiboMab format (as illustrated for example, in FIG. 13) may be particularly useful for RNA (e.g., ssRNA such as mRNA) that delivers a CLDN-18.2-targeting agent (e.g., a CLDN-18.2-targeted antibody agent) as described herein.

The present disclosure, among other things, provides insights and technologies for treating cancer, particularly, cancers that are associated with expression of Claudin-18.2 (CLDN-18.2). In some embodiments, the present disclosure provides technologies for treating a cancer selected from the group consisting of pancreatic cancers, gastric or gastro-esophageal cancers, biliary cancers, ovarian cancers, etc. In some embodiments, the present disclosure provides technologies for administration of therapy to locally advanced tumors. In some embodiments, the present disclosure provides technologies for treatment of unresectable tumors. In some embodiments, provided technologies provide technologies for treatment of metastatic tumors. Thus, for example, in some embodiments, provided therapy may be administered to a subject or population of subjects suffering from or susceptible to cancer (e.g., to a cancer selected from pancreatic cancers, gastric or gastro-esophageal cancers, biliary cancers, ovarian cancers, and/or otherwise involves one or more pancreatic, gastric, gastroesophageal, biliary, and/or ovarian tumors), which cancer may be or comprise one or more locally advanced tumors, one or more unresectable tumors and/or one or more metastases.

Zolbetuximab (development code IMAB362), which is a monoclonal antibody that targets isoform 2 of Claudin-18, has been under investigation for the treatment of gastrointestinal adenocarcinomas and pancreatic tumors. In the Phase 2a MONO trial with IMAB362 (NCT01197885), IMAB362 treatment emergent adverse events (“TEAE”s) occurred in 82% (n=44/54) of the patients; nausea (61%), vomiting (50%) and fatigue (22%) were the most frequent TEAEs. Grade 3 vomiting was reported in 12 patients (22%) and grade 3 nausea in eight patients (15%). These patients received the 600 mg/m² dose. The nausea and vomiting observed in such IMAB362 study were managed by pausing or slowing infusion of IMAB362 indicating that the AEs are Cₘₐₓ related (Türeci et al. 2019).

The present disclosure, among other things, provides an insight that administration of a nucleic acid such as RNA (e.g., ssRNA such as mRNA) encoding a CLDN-18.2-targeting agent, and in particular a CLDN-18.2-targeting antibody agent, and specifically IMAB362 may represent a particularly desirable strategy for CLDN-18.2-targeted therapy. Without wishing to be bound by any particular theory, the present disclosure proposes that such delivering modality may achieve one or more improvements such as effective administration with reduced incidence (e.g., frequency and/or severity) of TEAEs, and/or with improved relationship between efficacy level and TEAE level (e.g., improved therapeutic window) relative to those observed when a corresponding (e.g., encoded) protein (e.g., antibody) agent itself is administered. In particular, the present disclosure teaches that such improvements in particular may be achieved by delivering IMAB362 via administration of a nucleic acid, and in particular of RNA(s) (e.g., ssRNA(s) such as mRNA(s)) encoding it.

In some embodiments, the present disclosure, among other things, provides insights that mRNA(s) encoding an antibody agent (e.g., IMAB362) or a functional portion thereof that is/or formulated with lipid nanoparticles (LNP) for intravenous (IV) administration can be taken up by target cells (e.g., liver cells) for efficient production of the encoded antibody agent (e.g., IMAB362) at therapeutically relevant plasma concentrations, for example, as illustrated in FIG. 14 for the described RiboMab targeting CLDN-18.2.

In some embodiments, the present disclosure utilizes RiboMabs as CLDN-18.2-targeting agents. In some embodiments, such RiboMabs are antibody agents encoded by mRNA, e.g., engineered for minimal immunogenicity, and/or formulated in lipid nanoparticles (LNPs).

Moreover, the present disclosure, among other things, provides an insight that the capability of a CLDN-18.2-targeted antibody agent as described herein to induce antibody-dependent cellular cytotoxicity (ADCC) and/or complement-dependent cytotoxicity (CDC) against target cells (e.g., tumor cells) while leveraging immune system of recipient subjects can augment cytotoxic effect(s) of chemotherapy and/or other anti-cancer therapy. In some embodiments, such a combination therapy may prolong progression-free and/or overall survival, e.g., relative to the individual therapies administered alone and/or to another appropriate reference.

Without wishing to be bound by a particular theory, the present disclosure observes that certain chemotherapeutic agents, for example such as gemcitabine, oxaliplatin, and 5-fluorouracil were shown to upregulate existing CLDN-18.2 expression levels in pancreatic cancer cell lines; moreover, these agents were not observed to increase de novo expression in CLDN-18.2—negative cell lines. See, for example, Tureci et al. (2019) “Characterization of zolbetuximab in pancreatic cancer models” In Oncoimmunology 8 (1), pp. e1523096.

The present disclosure, among other things, provides an insight that CLDN-18.2-targeted therapy as described herein may be particularly useful and/or effective when administered to tumor(s) (e.g., tumor cells, subjects in whom such tumor(s) and/or tumor cell(s) are suspected and/or have been detected, etc.) characterized by (e.g., that have been determined to display and/or that are expected or predicted to display) elevated expression and/or activity of CLDN-18.2 expression in tumor cells (e.g., as may result or have resulted from exposure to one or more chemotherapeutic agents). Indeed, among other things, the present disclosure teaches that provided CLDN-18.2-targeted therapy (e.g., administration of a nucleic acid such as an RNA and, more particularly an mRNA encoding a CLDN-18.2-targeting antibody agent) as described herein may provide synergistic therapeutic when administered in combination with (e.g., to a subject who has received and/or is receiving or has otherwise been exposed to) one or more CDLN18.2-enhancing agents (e.g., one or more certain chemotherapeutic agents). Accordingly, in some embodiments, CLDN-18.2-targeted therapy as described herein can be useful in combination with other anti-cancer agents that are expected to and/or have been demonstrated to up-regulate CLDN-18.2 expression in tumor cells.

In some aspects, provided herein are pharmaceutical compositions targeting CLDN-18.2. In some embodiments, such a pharmaceutical composition comprises: (a) at least one single-stranded RNA (ssRNA) comprising one or more coding regions that encode an antibody agent that binds preferentially to a Claudin-18.2 (CLDN-18.2) polypeptide relative to a Claudin-18.1 (CLDN18.1) polypeptide (“CLDN-18.2-targeting antibody agent”); and (b) lipid nanoparticles; wherein the at least one single-stranded RNA is encapsulated within at least one of the lipid nanoparticles. In some embodiments, such a pharmaceutical composition can comprise and/or deliver one or more ssRNAs encoding an antibody that binds preferentially to CLDN-18.2 polypeptide relative to a CLND18.1 polypeptide. In some embodiments, such a pharmaceutical composition can comprise and/or deliver one or more ssRNAs encoding an antigen binding fragment that that binds preferentially to CLDN-18.2 polypeptide relative to a CLND18.1 polypeptide.

In some embodiments, an antibody agent that targets CLDN-18.2 (and may be encoded by an RNA such as an ssRNA, e.g., an mRNA as described herein) specifically binds to a first extracellular domain (ECD1) of a CLDN-18.2 polypeptide. For example, in some embodiments, such an antibody agent specifically binds to an epitope of ECD1 that is exposed in cancer cells.

In some embodiments, at least one ssRNA (e.g., mRNA) encodes a variable heavy chain (V_{H}) domain of a CLDN-18.2-targeting antibody agent and a variable light chain (V_{L}) domain of the antibody agent. In some embodiments, such V_{H} domain(s) and V_{L} domain(s) of a CLDN-18.2-targeting antibody agent may be encoded by a single ssRNA construct; alternatively in some embodiments they may be encoded separately by at least two individual ssRNA constructs. For example, in some embodiments, an ssRNA as utilized herein comprises two or more coding regions, which comprises a heavy chain-coding region that encodes at least a V_{H} domain of the antibody agent; and a light chain-coding region that encodes at least a V_{L} domain of the antibody agent. In alternative embodiments, a pharmaceutical composition may comprise: (i) a first ssRNA comprising a heavy chain-coding region that encodes at least a V_{H} domain of the antibody agent; and (ii) a second ssRNA comprising a light chain-coding region that encodes at least a V_{L} domain of the antibody agent.

In some embodiments, a heavy chain-coding region can further encode a constant heavy chain (C_{H}) domain; and/or a light chain-coding region can further encode a constant light chain (C_{L}) domain. For example, in some embodiments, a heavy chain-coding region may encode a V_{H} domain, a C_{H1} domain, a C_{H2} domain, and a C_{H3} domain of an antibody agent in an immunoglobulin G (IgG) form; and/or a light chain-coding region may encode a V_{L} domain and a C_{L} domain of an antibody agent in an IgG form. In some embodiments, an antibody agent in an IgG form is IgG1.

In some embodiments, a heavy chain-coding region of an ssRNA consists of or comprises a nucleotide sequence that encodes a full-length heavy chain of Zolbetuximab or Claudiximab. In some embodiments, a light chain-coding region of an ssRNA consists of or comprises a nucleotide sequence that encodes a full-length light chain of Zolbetuximab or Claudiximab.

In some embodiments, ssRNA(s) that encode a CLDN-18.2-targeting antibody agent may comprise a secretion signal-encoding region. In some embodiments, such a secretion signal-encoding region allows a CLDN-18.2-targeting antibody agent encoded by one or more RNAs to be secreted upon translation by cells, e.g., present in a subject to be treated, thus yielding a plasma concentration of a biologically active CLDN-18.2-targeting antibody agent.

In some embodiments, ssRNA(s) that encode a CLDN-18.2-targeting antibody agent may comprise at least one non-coding sequence element (e.g., to enhance RNA stability and/or translation efficiency). Examples of non-coding sequence elements include but are not limited to a 3′ untranslated region (UTR), a 5′ UTR, a cap structure for co-transcriptional capping of mRNA, a poly adenine (polyA) tail, and any combinations thereof. For example, in some embodiments, ssRNA(s) (e.g., a first ssRNA and/or a second ssRNA) each independently comprise, in a 5′ to 3′ direction: (a) a 5′UTR-coding region; (b) a secretion signal-coding region; (c) the heavy chain-coding region; (d) a 3′ UTR-coding region; and (e) a polyA tail-coding region. In some embodiments, a polyA tail-coding region included in an ssRNA is or comprises a modified polyA sequence.

In some embodiments, ssRNA(s) that encode a CLDN-18.2-targeting antibody agent may comprise a 5′ cap.

In some embodiments, ssRNA(s) that encode a CLDN-18.2-targeting antibody agent may comprise at least one modified ribonucleotide. For example, in some embodiments, at least one of A, U, C, and G ribonucleotide of ssRNA(s) s may be replaced by a modified ribonucleotide. In some embodiments, such a modified ribonucleotide may be or comprise pseudouridine.

In some embodiments where a pharmaceutical composition comprises a first ssRNA encoding a variable heavy chain (V_{H}) domain of a CLDN-18.2-targeting antibody agent and a second ssRNA encoding a variable light chain (V_{L}) domain of the antibody agent, such a first ssRNA and a second ssRNA may be present in a molar ratio of about 1.5:1 to about 1:1.5. In some embodiments, such a first ssRNA and a second ssRNA may be present in a weight ratio of 3:1 to 1:1. In some embodiments, such a first ssRNA and a second ssRNA may be present in a weight ratio of about 2:1.

In some embodiments, RNA content (e.g., one or more ssRNAs encoding a CLDN-18.2-targeting antibody agent) of a pharmaceutical composition described herein is present at a concentration of 0.5 mg/mL to 1.5 mg/mL.

In some embodiments, lipid nanoparticles provided in pharmaceutical compositions described herein are liver-targeting lipid nanoparticles. In some embodiments, lipid nanoparticles provided in pharmaceutical compositions described herein are cationic lipid nanoparticles. In some embodiments, lipid particles provided in pharmaceutical compositions described herein may have an average size of about 50-150 nm.

In some embodiments, lipids that form the lipid nanoparticles comprise: a polymer-conjugated lipid; a cationic lipid; and a neutral lipid. In some such embodiments, a polymer-conjugated lipid is be present in about 1-2.5 mol % of the total lipids; a cationic lipid is present in 35-65 mol % of the total lipids; and a neutral lipid is present in 35-65 mol % of the total lipids.

Various lipids (including, e.g., polymer-conjugated lipids, cationic lipids, and neutral lipids) are known in the art and can be used herein to form lipid nanoparticles, e.g., lipid nanoparticles targeting a specific cell type (e.g., liver cells). In some embodiments, a polymer-conjugated lipid included in pharmaceutical compositions described herein may be a PEG-conjugated lipid (e.g., 2-[(polyethylene glycol)-2000]-N,N-ditetradecylacetamide or a derivative thereof). In some embodiments, a cationic lipid included in pharmaceutical compositions described herein may be ((3-hydroxypropyl)azanediyl)bis(nonane-9,1-diyl) bis(2-butyloctanoate) or a derivative thereof. In some embodiments, neutral lipid included in pharmaceutical compositions described herein may be or comprise a phospholipid or derivative thereof (e.g., 1,2-Distearoyl-sn-glycero-3-phosphocholine (DPSC)) and/or cholesterol.

In some embodiments, a pharmaceutical composition described herein may further comprise one or more additives, for example, in some embodiments that may enhance stability of such a composition under certain conditions. For example, in some embodiments, a pharmaceutical composition may further comprise a cryoprotectant (e.g., sucrose) and/or an aqueous buffered solution, which may in some embodiments include one or more salts (e.g., sodium salts).

In some embodiments, a pharmaceutical composition described herein may further comprises one or more active agents other than RNA (e.g., an ssRNA such as an mRNA) encoding a CLDN-18.2-targeting agent (e.g., antibody agent). For example, in some embodiments, such an other active agent may be or comprise a chemotherapeutic agent. An exemplary chemotherapeutic agent may be or comprise a chemotherapeutic agent indicated for treatment of pancreatic cancer.

In some embodiments, pharmaceutical compositions described herein can be taken up by target cells for production of an encoded CLDN-18.2-targeting antibody agent at therapeutically relevant plasma concentrations. In some embodiments, such pharmaceutical compositions described herein can induce antibody-dependent cellular cytotoxicity (ADCC) and complement-dependent cytotoxicity (CDC) against target cells (e.g., tumor cells).

Accordingly, another aspect of the present disclosure relates to methods of using pharmaceutical compositions described herein. For example, one aspect provided herein relates to a method comprising administering a provided pharmaceutical composition to a subject suffering from a CLDN-18.2-positive solid tumor. Examples of a CLDN-18.2-positive solid tumor are but are not limited to a biliary tract tumor, a gastric tumor, a gastro-esophageal tumor, an ovarian tumor, a pancreatic tumor, and a tumor that expresses or exhibits a certain level of a CLDN-18.2 polypeptide. In some embodiments, a CLDN-18.2-positive tumor may be characterized in that ≥50% of tumor cells show ≥2+ CLDN-18.2 protein staining intensity as assessed by an immunohistochemistry assay in formalin-fixed, paraffin-embedded neoplastic tissue from a subject to be administered. In some embodiments, a subject suffering from a CLDN-18.2-positive solid tumor may have a locally advanced, unresectable, or metastatic tumor. In some embodiments, a subject suffering from a CLDN-18.2 positive solid tumor may have received a pre-treatment sufficient to increase CLDN-18.2 level such that his/her solid tumor is characterized as a CLDN-18.2-positive solid tumor.

In some embodiments, a pharmaceutical composition described herein may be administered as monotherapy. In some embodiments, a pharmaceutical composition may be administered as part of combination therapy comprising such a pharmaceutical composition and a chemotherapeutic agent. Accordingly, in some embodiments, a subject who is receiving a provided pharmaceutical composition has received a chemotherapeutic agent. In some embodiments, a subject who is receiving a provided pharmaceutical composition is administered a chemotherapeutic agent such that such a subject is receiving both as a combination therapy. In some embodiments, a provided pharmaceutical composition and a chemotherapeutic agent may be administered concurrently or sequentially. For example, in some embodiments, a chemotherapeutic agent may be administered after (e.g., at least four hours after) administration of a provided pharmaceutical composition.

In some embodiments, technologies provided herein are useful for treatment of a CLDN-18.2 positive pancreatic tumor. In some embodiments involving administration of a provided pharmaceutical composition to a subject suffering from a CLDN-18.2-positive pancreatic tumor, such a subject may be receiving such a provided composition as a monotherapy or as part of a combination therapy comprising such a provided pharmaceutical composition and a chemotherapeutic agent indicated for treatment of pancreatic tumor. In some embodiments, such a chemotherapeutic agent may be or comprise gemcitabine and/or paclitaxel (e.g., nab-paclitaxel). In some embodiments, such a chemotherapeutic agent may be or comprise FOLFIRINOX, which is a combination of cancer drugs including: folinic acid (FOL), fluorouracil (F), irinotecan (IRIN), and oxalipatin (OX).

In some embodiments, technologies provided herein are useful for treatment of a CLDN-18.2 positive biliary tract tumor. In some embodiments involving administration of a provided pharmaceutical composition to a subject suffering from a CLDN-18.2-positive biliary tract tumor, such a subject may be receiving such a provided composition as a monotherapy or as part of a combination therapy comprising such a provided pharmaceutical composition and a chemotherapeutic agent indicated for treatment of biliary tract tumor. In some embodiments, such a chemotherapeutic agent may be or comprise gemcitabine and/or cisplatin.

Pharmaceutical compositions and methods described herein may be applicable to a subject of any age suffering from a CLDN-18.2 positive solid tumor. In some embodiments, a subject suffering from a CLDN-18.2 positive solid tumor is an adult subject.

Pharmaceutical compositions described herein may be administered to a subject in need thereof by appropriate methods known in the art. For example, in some embodiments, a provided pharmaceutical composition may be administered to a subject suffering from a CLDN-18.2 positive solid tumor by intravenous injection.

Dosage of pharmaceutical compositions described herein may vary with a number of factors including, e.g., but not limited to body weight of a subject to be treated, cancer types and/or cancer stages, and/or monotherapy or combination therapy. In some embodiments, a pharmaceutical composition described herein is administered to a subject suffering from a CLDN-18.2 positive solid tumor in at least one or more (including, e.g., at least two, at least three, at least four, at least five, at least six, at least seven, at least eight, or more) dosing cycles. In some embodiments, each dosing cycle may be a three-week dosing cycle. In some embodiments, a pharmaceutical composition described herein is administered is at least one dose per dosing cycle. In some embodiments, a dosing cycle involves administration of a set number and/or pattern of doses; in some embodiments, a dosing cycle involves administration of a set cumulative dose, e.g., over a particular period of time, and optionally via multiple doses, which may be administered, for example, at set interval(s) and/or according to a set pattern. In some embodiments, each dose or a cumulative dose of a pharmaceutical composition described herein may comprise one or more ssRNAs encoding a CLDN-18.2-targeting antibody agent (whether encoded by a single ssRNA or two or more ssRNAs) in an amount within a range of 0.1 mg/kg to 5 mg/kg body weight of a subject to be administered.

Another aspect of the present disclosure relates to certain improvement in a method of delivering a CLDN-18.2-targeting antibody agent for cancer treatment in a subject, which method comprises administering to a cancer subject a provided pharmaceutical composition. In some embodiments, pharmaceutical compositions described herein may achieve one or more improvements such as effective administration with reduced (e.g., frequency and/or severity) of TEAEs, and/or with improved relationship between efficacy level and TEAE level (e.g., improved therapeutic window) relative to those observed when a corresponding (e.g., encoded) protein (e.g., antigen) agent itself is administered. In particular, the present disclosure teaches that such improvements in particular may be achieved by delivering IMAB362 via administration of a nucleic acid, and in particular of RNA(s) (e.g., ssRNA(s) such as mRNA(s))) encoding it.

Methods of producing a CLDN-18.2-targeting antibody agent are also within the scope of the present disclosure. In some embodiments, a method of producing a CLDN-18.2-targeting antibody agent comprises administering to cells a composition comprising at least one ssRNA (e.g., ones as described herein) comprising one or more coding regions that encode a CLDN-18.2-targeting antibody agent so that such cells express and secrete a CLDN-18.2-targeting antibody agent encoded by such ssRNA(s). In some embodiments, cells to be administered or targeted are or comprise liver cells.

In some embodiments, cells are present in a cell culture.

In some embodiments, cells are present in a subject. In some such embodiments, a pharmaceutical composition described herein may be administered to a subject in need thereof. In some embodiments, such a pharmaceutical composition may be administered to a subject such that a CLDN-18.2-targeting antibody agent is produced at a therapeutically relevant plasma concentration. In some embodiments, a therapeutically relevant plasma concentration is sufficient to mediate cancer cell death through antibody-dependent cellular cytotoxicity (ADCC). For example, in some embodiments, a therapeutically relevant plasma concentration is 0.3-28 μg/mL.

Among other things, the present disclosure also provides methods of characterizing one or more features of an ssRNA or composition thereof, which ssRNA encodes part or all of an antibody agent. In some embodiments, a method comprising a step of: determining one or more features of an antibody agent expressed from at least one mRNA introduced into cells, wherein such at least one mRNA comprises one or more of features of at least one or more ssRNA comprising a coding region that encodes an antibody agent that binds preferentially to a Claudin-18.2 (CLDN-18.2) polypeptide relative to a Claudin-18.1 polypeptide, wherein such one or more features comprises: (i) protein expression level of an antibody agent; (ii) binding specificity of an antibody agent to CLDN-18.2; (iii) efficacy of an antibody agent to mediate target cell death through ADCC; and (iv) efficacy of an antibody agent to mediate target cell death through complement dependent cytotoxicity (CDC).

In some embodiments, provided herein is a method of characterizing a pharmaceutical composition targeting CLDN-18.2. Such a method comprises steps of: (a) contacting cells with at least one composition or pharmaceutical composition described herein (which encodes part or all of a CLDN-18.2-targeting antibody agent); and detecting an antibody agent produced by the cells. In some embodiments, the cells may be or comprise liver cells.

In some embodiments, such a method may further comprise determining one or more features of an antibody agent expressed from one or more ssRNAs described herein, wherein such one or more features comprises: (i) protein expression level of the antibody agent; (ii) binding specificity of the antibody agent to a CLDN-18.2 polypeptide; (iii) efficacy of the antibody agent to mediate target cell death through ADCC; and (iv) efficacy of the antibody agent to mediate target cell death through complement dependent cytotoxicity (CDC). In some embodiments, a step of determining one or more features of an antibody agent expressed from one or more ssRNAs described herein may comprise comparing such features of the CLDN-18.2-targeting antibody agent with that of a reference CLDN-18.2-targeting antibody.

In some embodiments, a step of determining one or more features of an antibody agent expressed from one or more ssRNAs described herein may comprise assessing the protein expression level of the antibody agent above a threshold level. For example, in some embodiments, a threshold level corresponds to a therapeutically relevant plasma concentration.

In some embodiments, a step of determining one or more features of an antibody agent expressed from one or more ssRNAs described herein may comprise assessing binding of the antibody agent to a CLDN-18.2 polypeptide. In some embodiments, such binding assessment may comprise determining binding of the antibody agent to a CLDN-18.2 polypeptide relative to its binding to a CLDN18.1 polypeptide. In some embodiments, such binding assessment may comprise determining a binding preference profile of the antibody agent at least comparable to that of a reference CLDN-18.2-targeting antibody. For example, in some embodiments, a reference CLDN-18.2-targeting antibody is Zolbetuximab or Claudiximab.

In some embodiments, a provided method of characterizing a pharmaceutical composition targeting CLDN-18.2 or components thereof may further comprise characterizing an antibody agent expressed from one or more ssRNAs described herein as a CLDN-18.2-targeting antibody agent if the antibody agent comprises the following features: (a) protein level of the antibody agent expressed by the cells above a threshold level; (b) preferential binding of the antibody agent to CLDN-18.2 relative to CLDN18.1; and (c) killing of at least 50% target cells (e.g., cancer cells) mediated by ADCC and/or CDC.

In some embodiments, a provided method of characterizing a pharmaceutical composition targeting CLDN-18.2 or components thereof may further comprise characterizing an antibody agent expressed from one or more ssRNAs described herein as a Zolbetuximab or Claudiximab-equivalent antibody if tested features of the antibody are at least comparable to that of Zolbetuximab or Claudiximab.

In some embodiments involving a step of determining one or more features of an antibody agent expressed from one or more ssRNAs described herein, such a step may comprise determining one or more of the following features:
whether, when assessed 48 hours after contacting or administering, cells express a CLDN-18.2-targeting antibody agent encoded by at least one ssRNA;
whether the antibody agent expressed by the cells binds preferentially to a CLDN-18.2 polypeptide relative to a CLDN18.1 polypeptide;
whether the antibody agent expressed by the cells exhibit comparable target specificity to CLDN-18.2 as observed in a flow cytometric binding assay with a reference CLDN-18.2-targeting monoclonal antibody;
whether, when assessed 48 hours after incubating immune effector cells (e.g., PBMC cells) and CLDN-18.2 positive cells or CLDN-18.2 negative control cells in the presence of the antibody agent, the CLDN-18.2 positive cells, not the control cells, were lysed;
whether the antibody agent expressed by the cells exhibit at least comparable ADCC profile of targeted CLDN-18.2 positive cells as observed with a reference CLDN-18.2-targeting monoclonal antibody in the same concentration; and
whether, when assessed 2 hours after incubating CLDN-18.2 positive cells or CLDN-18.2 negative control cells with human serum in the presence of the antibody agent, the CLDN-18.2 positive cells, not the control cells, were lysed.

In some embodiments, cells used in provided methods of characterizing a pharmaceutical composition targeting CLDN-18.2 or components thereof are present in vivo, e.g., in a subject (e.g., a mammalian subject such as a mammalian non-human subject, e.g., a mouse or monkey subject). In some such embodiments, a step of determining one or more features of an antibody agent expressed from one or more ssRNAs described herein may include determining antibody level in one or more tissues in such a subject. In some embodiments, such a method of characterizing may further comprise administering a composition or pharmaceutical composition described herein to a group of animal subjects each bearing a human CLDN-18.2 positive xenograft tumor to determine anti-tumor activity, if such a composition or pharmaceutical composition is characterized as a CLDN-18.2-targeting antibody agent.

Also within the scope of the present disclosure includes a method of manufacture, which comprises steps of:
(A) determining one or more features of an ssRNA or composition thereof, which ssRNA encodes part or all of an antibody agent, which one or more features are selected from the group consisting of:
   (i) length and/or sequence of the ssRNA;
   (ii) integrity of the ssRNA;
   (iii) presence and/or location of one or more chemical moieties of the ssRNA;
   (iv) extent of expression of the antibody agent when the ssRNA is introduced into a cell;
   (v) stability of the ssRNA or composition thereof;
   (vi) level of antibody agent in a biological sample from an organism into which the ssRNA has been introduced;
   (vii) binding specificity of the antibody agent expressed from the ssRNA, optionally to CLDN-18.2 and optionally relative to CLDN18.1;
   (viii) efficacy of the antibody agent to mediate target cell death through ADCC;
   (ix) efficacy of the antibody agent to mediate target cell death through complement dependent cytotoxicity (CDC);
   (x) lipid identity and amount/concentration within the composition;
   (xi) size of lipid nanoparticles within the composition;
   (xii) polydispersity of lipid nanoparticles within the composition;
   (xiii) amount/concentration of the ssRNA within the composition;
   (xiv) extent of encapsulation of the ssRNA within lipid nanoparticles; and
   (xv) combinations thereof;
(B) comparing such one or more features of the ssRNA or composition thereof with that of an appropriate reference standard; and
(C) (i) designating the ssRNA or composition thereof for one or more further steps of manufacturing and/or distribution if the comparison demonstrates that the ssRNA or composition thereof meets or exceeds the reference standard; or
   (ii) taking an alternative action if the comparison demonstrates that the ssRNA or composition thereof does not meet or exceed the reference standard.

In some embodiments of a method of manufacture, when an ssRNA (e.g., ones described herein) is assessed and one or more features of the ssRNA meets or exceeds an appropriate reference standard, such an ssRNA is designated for formulation, e.g., in some embodiments involving formulation with lipid particles described herein.

In some embodiments of a method of manufacture, when a composition comprising an ssRNA (e.g., ones described herein) is assessed and one or more features of the composition meets or exceeds an appropriate reference standard, such a composition is designated for release and/or distribution of the composition.

In some embodiments of a method of manufacture, when an ssRNA (e.g., ones described herein) is designated for formulation, and/or a composition comprising an ssRNA (e.g., ones described herein) is designated for release and/or distribution of the composition, such a method may further comprise administering the formulation and/or composition to a group of animal subjects each bearing a human CLDN-18.2 positive xenograft tumor to determine anti-tumor activity.

Provided herein is also a method of determining a dosing regimen of a pharmaceutical composition targeting CLDN-18.2. For example, in some embodiments, such a method comprises steps of: (A) administering a pharmaceutical composition (e.g., ones described herein) to a subject suffering from a CLDN-18.2 positive solid tumor under a pre-determined dosing regimen; (B) monitoring or measuring tumor size of the subject periodically over a period of time; (C) evaluating the dosing regimen based on the tumor size measurement(s). For example, a dose and/or dosage frequency can be increased if reduction in tumor size after the administration of a pharmaceutical composition (e.g., ones described herein) is not therapeutically relevant; or a dose and/or dosage frequency can be decreased if reduction in tumor size after the administration of a pharmaceutical composition (e.g., ones described herein) is therapeutically relevant, but adverse effect (e.g., toxicity effect) is shown in the subject. If reduction in tumor size after the administration of a pharmaceutical composition (e.g., ones described herein) is therapeutically relevant, and no adverse effect (e.g., toxicity effect) is shown in the subject, no changes is made to a dosage regimen.

In some embodiments, such a method of determining a dosing regimen of a pharmaceutical composition targeting CLDN-18.2 may be performed in a group of animal subjects (e.g., mammalian non-human subjects) each a bearing a human CLDN-18.2 positive xenograft tumor. In some such embodiments, a dose and/or dosage frequency can be increased if less than 30% of the animal subjects exhibit reduction in tumor size after the administration of a pharmaceutical composition (e.g., ones described herein) and/or extent of reduction in tumor size exhibited by the animal subjects is not therapeutically relevant; or a dose and/or dosage frequency can be decreased if reduction in tumor size after the administration of a pharmaceutical composition (e.g., ones described herein) is therapeutically relevant, but significant adverse effect (e.g., toxicity effect) is shown in at least 30% of the animal subjects. If reduction in tumor size after the administration of a pharmaceutical composition (e.g., ones described herein) is therapeutically relevant, and no significant adverse effect (e.g., toxicity effect) is shown in the animal subjects, no changes is made to a dosage regimen.

### BRIEF DESCRIPTION OF THE DRAWINGS

FIG. 1 shows that a CLDN-18.2-targeting antibody (RiboMab01) encoded by two RNAs encoding a heavy chain and a light chain, respectively, of a CLDN-18.2-targeting antibody is expressed in primary human hepatocytes and CHO-K1 cells. (Panel A) Primary human hepatocytes were lipofected with 0.22-55.50 μg/mL a composition comprising two or more RNAs encoding heavy chain and light chain, respectively, of a CLDN-18.2-targeting antibody (RB_RMAB01). (Left) ELISA analyses of RiboMab01 concentrations 48 hours post transfection. (Right) Western Blot analysis of cell culture supernatant from indicated lipofections. Recombinant purified IMAB362 served as reference for Western Blot analysis. Analysis was performed under non-reducing conditions and with HRP-conjugated anti-human antibodies. A mixture of Fcγ-fragment specific and anti-kappa light chain specific antibodies was used for detection of full length IgG, free heavy (HC) and free light chains (LC). Supernatant of untransfected primary human hepatocytes served as mock control. (Panel B) CHO-K1 cells were lipofected with 2.00-182.00 ng/mL RB_RMAB01. RiboMab01 concentration determined via ELISA 48 hours post transfection are shown. Error bars are standard errors of the mean (n=3).

FIG. 2 shows that RiboMab01 binds target specific to CLDN-18.2. Targeted binding of RiboMab01 to CLDN-18.2 was determined by flow cytometric binding assays visualized using a fluorescently labeled antibody directed against the F(ab′)2 fragment of human IgG (H+L). A dilution row of RiboMab01-containing CHO-K1 cell culture supernatant (Panels A and B, left) or IMAB362 reference protein (Panels A and B, right) was incubated with 5×10⁵ (Panel A) CLDN-18.2+ or (Panel B) CLDN18.1+HEK293 transfectants.

FIG. 3 shows high target specific cell cytotoxicity mediated by in vitro expressed RiboMab01. RiboMab01-containing cell culture supernatant from CHO-K1 cells lipofected with RB_RMAB01 was subjected to (Panel A) ADCC and (Panel B) CDC assays. (Panel A) For ADCC assays, CLDN-18.2+ NUG-C4 transfectants served as target cells and CLDN-18.2-negative MDA-MB-231 cells as control cells. Human PBMCs of three different healthy donors were utilized as effector cells (E:T ratio 30:1). Target or control and effector cells were incubated for 48 hours with the indicated RiboMab01 and IMAB362 reference protein concentrations. Specific cell lysis as determined in a luciferase-based assay is shown. (Panel B) For CDC assays, CLDN-18.2+ CHO-K1 transfectants (solid lines) served as target cells and CLDN-18.2-negative CHO-K1 (dotted lines) as control cells. Target and control cells were incubated with human serum and RiboMab01 concentrations as indicated for 2 hours. CDC determined in a luciferase-based assay is shown. Error bars are standard errors of the mean (n=3).

FIG. 4 shows specific tumor cell lysis mediated by RiboMab01 generated in mice. Plasma of mice dosed with five repetitive injections of either 1 μg (˜0.04 mg/kg), 3 μg (˜0.10 mg/kg), 10 μg (˜0.40 mg/kg) and 30 μg (˜1.20 mg/kg) RB_RMAB01 or 80 μg (˜3.20 mg/kg) of IMAB362 was sampled 24 hours post 5th injection and directed to luciferase-based ex vivo ADCC assays. Plasma of untreated mice spiked with IMAB362 served as assay reference. CLDN-18.2+ NUG-C4 transfectants served as target and human PBMCs as effector cells. (Panel A) RiboMab01 mediated ADCC of NUG-C4 cells after 48 hours incubation with 1% of plasma is shown. (Panel B) No unspecific lysis on target-negative MDA-MB-231 cells. Error bars are standard errors of the mean (n=3).

FIG. 5 shows that RiboMab01 expressed by non-human primates mediates dose-dependent ADCC. Non-Human Primates (NHP) received three repetitive doses of 0.1, 0.4 or 1.6 mg/kg RB_RMAB01 once weekly. RiboMab01-containing serum of all monkeys sampled 24 hours (black bars) and 168 hours (white bars) post 1st injection was directed to luciferase-based ex vivo ADCC assays. CLDN-18.2+ NUG-C4 transfectants served as target cells. Human PBMCs from two different healthy donors (24 h, donor 1, 168 h, donor 2) served as effector cells. (Panel A) RiboMab01-mediated ADCC of NUG-C4 cells after 48 hours incubation is shown. (Panel B) Unspecific lysis on target-negative MDA-MB-231 cells is shown. Error bars are standard error of the mean (n=3). (Panel C) Serum of NHP No. 14 (1.6 mg/kg RB_RMAB01, RiboMab01 serum concentration 232 μg/mL) collected 48 hours after the 3rd dosing was used for a luciferase-based ex vivo ADCC assays. CLDN-18.2+ NUG-C4 transfectants (solid lines) served as target cells, CLDN-18.2-negative MDA-MB-231 cells (dotted lines) as control cells. Human PBMCs of a healthy donor served as effector cells. ADCC of NUG-C4 cells mediated by RiboMab01-containing serum (solid red line) or by the recombinant—IMAB362 reference protein (solid black line)—with an EC50 of 66 pM and 151 pM respectively—is shown. Dotted red and black lines represent weak unspecific lysis on MDA-MB-231 control cells. Incubation time was 48 hours. Error bars are standard errors of the mean (n=3).

FIG. 6 shows that systemic availability of RiboMab01 mediates tumor growth inhibition in vivo. Mice bearing subcutaneous CLDN-18.2+ NCI-N87 xenograft tumors received IV injections of 1 μg (˜0.04 mg/kg), 3 μg (˜0.10 mg/kg), 10 μg (˜0.40 mg/kg) and 30 μg (˜1.20 mg/kg) RB_RMAB01, 800 μg (˜32 mg/kg) IMAB362 reference protein, 30 μg (˜1.20 mg/kg) luciferase mRNA or saline only on test days 15, 22, 29, 36, 43 and 50 post tumor cell inoculation. Median tumor growth of treatment and control groups is shown. Dotted lines indicate injections. Significance was calculated by Two-way ANOVA. Ns indicates not significant.

FIG. 7 shows concentration-time profile of RiboMab01 in mouse serum after single dosing. Balb/cJRj mice received a single IV injection of 1 μg (0.040 mg/kg), 3 μg (˜0.10 mg/kg), 10 μg (˜0.40 mg/kg) or 30 μg (˜1.20 mg/kg) RB_RMAB01 drug product and μg (˜1.60 mg/kg) IMAB362 reference protein. Plasma was sampled 6, 24, 96, 168, 264, 336 and 504 hours post administration. RiboMab01 concentrations in plasma measured via ELISA are shown. Error bars are standard errors of the mean (n=3).

FIG. 8 shows concentration-time profile of RiboMab01 in rat serum after single dosing. RjHan:Wister rats received a single IV injection of 0.04, 0.10, 0.40 or 1.20 mg/kg of RB_RMAB01 and 3.60 mg/kg of IMAB362 reference protein. Plasma was sampled 2, 6, 8, 10, 22, 24, 27, 30, 48, 72, 96, 168, 216, 264 and 336 hours post administration. RiboMab01 concentrations in plasma measured via ELISA are shown. Error bars are standard errors of the mean (n=3).

FIG. 9 shows kinetics of RB_RMAB01 expression in mice after weekly injection. Balb/cJRj mice received IV injections of 1 μg (˜0.04 mg/kg), 3 μg (˜0.10 mg/kg), 10 μg (˜0.40 mg/kg) or 30 μg (˜1.20 mg/kg) RB_RMAB01 and 80 μg (˜3.20 mg/kg) IMAB362 reference protein at test days 1, 8, 15, 21 and 29. Plasma was sampled 24 hours pre- and 24 hours post-dosing. RiboMab01 concentrations in plasma measured via ELISA are shown. Dotted lines indicate injections. Error bars are standard errors of the mean (n=3).

FIG. 10 shows kinetics of RB_RMAB01 expression after repetitive dosing in NHP. NHP received IV injections of 0.1, 0.4 or 1.6 mg/kg RB_RMAB01 at test days 1, 8 and Plasma was sampled 6, 24, 48, 72, 96 and 168 hours post 1st and 3rd dosing and 48, 72 and 168 hours post 2nd dosing as well as 264, 336 and 504 hours post 3rd dosing. RiboMab01 concentrations in plasma measured via ELISA are shown. Error bars are standard errors of the mean (n=3).

FIG. 11 shows liver targeting of LNP formulated mRNA in vivo. Mice received a single IV injection of LNP formulated firefly luciferase mRNA. Bioluminescence was monitored 6, 24, 48, 72 and 144 hours after administration. (Panel A) Bioluminescent images 6 hours post administration are shown for (left) individual mice in ventral position (n=5) and (right) single organs of mice #1 and 2. (Panel B) Quantification of luciferase signals (photons/second) is shown for all time points of analysis (n=5 or 3, mean). LN indicates lymph nodes.

FIG. 12 illustrates exemplary embodiments of RNA technology useful for encoding various antibody agent formats (“RiboMab”) and formulations thereof as well as its applications. (Panel A) The RiboMab® platform is applicable to provide RNA constructs encoding various antibody formats, including, e.g., but not limited to monospecific antibody IgG, bispecific antibody bi-(scFv)₂, and bispecific antibody Fab-(svFv)₂. (Panel B) In some embodiments, therapeutic antibodies such as IgG can be encoded by purified mRNA comprising modified ribonucleotides (e.g., uridines replaced by pseudouridines) mRNA and encapsulated in lipid nanoparticles (mRNA/LNP). Such an mRNA construct may further comprise one or more non-coding sequence elements (e.g., to enhance RNA stability and/or translation efficiency). In some embodiments, exemplary non-coding sequence elements include but are not limited to a cap structure, 5′ UTR, 3′ UTR, a polyadenyl tail, and any combinations thereof. In some embodiments, lipid nanoparticles may comprises a conjugated lipid (e.g., PEG-conjugated lipid), a cationic lipid, and a neutral helper lipid. Such mRNA/LNP drug product formulation can be administered to a subject in vivo such that the mRNA is translated in vivo to express an antibody. (Panel C) The patient's own body cells administered with mRNA/LNP drug product formulations described herein are capable to produce active drug encoded by mRNA (e.g., IgG RiboMab). For example, in some embodiments, upon IV injection, antibody-encoding mRNA/LNP are internalized and translated by liver cells, yielding systemic plasma concentrations of the biologically active RiboMab. Abbreviations: A30L70, Poly(A) tail, measuring 100 adenosines abrogated by a linker at position 30; bi, bispecific; C, C-terminus; CDS, coding sequence; CH, constant heavy domain; CL, constant light domain; Fab, antigen-binding fragment; IgG, immunoglobulin G; LNP, lipid nanoparticle; m1Ψ, 1-methylpseudouridine; N, N-terminus; scFv, single-chain variable fragment; TAA, tumor-associated antigen; UTR, untranslated region; VH, variable heavy domain; VL, variable light domain.

FIG. 13 is a schematic representation of exemplary RNA constructs encoding a heavy chain (HC) and a light chain (LC), respectively, of an antibody agent. As presented in FIG. 13, such HC- and LC-encoding RNA constructs form an RNA composition (RB_RMAB01), which in some embodiments may be formulated into lipid nanoparticles to form a RNA/LNP drug product formulation. Abbreviations: Poly A, poly adenine tail; CH, constant heavy domain; CL, constant light domain; Sec, secretion signal; UTR, untranslated region; VH, variable heavy domain; VL, variable light domain

FIG. 14 is a graph showing dose-exposure correlation of RB_RMAB01 in cynomolgus monkey at tₘₐₓ. Cynomolgus monkeys (n=3) received IV injections of 0.1, 0.4 or 1.6 mg/kg RB_RMAB01. Dose-dependent RiboMab01 concentrations (mean, n=3) in plasma measured via ELISA at Cmax are depicted. A green line indicates a dose that can be administered to a human subject and its corresponding anticipated serum concentration.

FIG. 15 is an example electropherogram of an exemplary RNA mixture comprising a first RNA encoding a heavy chain (HC) of an antibody and a second RNA encoding a light chain (LC) of the antibody. The electropherogram depicts two peaks for LC and HC, respectively. A: area under the peak, h: height of the peak.

### CERTAIN DEFINITIONS

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Associated with: Two events or entities are “associated” with one another, as that term is used herein, if the presence, level and/or form of one is correlated with that of the other. For example, a particular biological phenomenon (e.g., expression of CLDN-18.2) is considered to be associated with a particular disease, disorder, or condition (e.g., cancer), if its presence correlates with incidence of and/or susceptibility of the disease, disorder, or condition (e.g., across a relevant population), or likelihood of responsiveness to a treatment.

Blood-derived sample: The term “blood-derived sample,” as used herein, refers to a sample derived from a blood sample (i.e., a whole blood sample) of a subject in need thereof. Examples of blood-derived samples include, but are not limited to, blood plasma (including, e.g., fresh frozen plasma), blood serum, blood fractions, plasma fractions, serum fractions, blood fractions comprising red blood cells (RBC), platelets, leukocytes, etc., and cell lysates including fractions thereof (for example, cells, such as red blood cells, white blood cells, etc., may be harvested and lysed to obtain a cell lysate). In some embodiments, a blood-derived sample that is used for characterization described herein is a plasma sample.

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. In some embodiments, cancer may be characterized by a solid tumor. In some embodiments, cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, ovarian cancer, breast cancer, glioblastomas, colorectal cancer, gastro-intestinal cancers and nervous system cancers, benign lesions such as papillomas, and the like.

Cap: As used herein, the term “cap” refers to a structure comprising or essentially consisting of a nucleoside-5 ‘-triphosphate that is typically joined to a 5’-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template, wherein, in addition to the GTP, an IVT system also contains a dinucleotide cap analog (including, e.g., a m7GpppG cap analog or an N7-methyl, 2′-O-methyl-GpppG ARCA cap analog or an N7-methyl, 3′-O-methyl-GpppG ARCA cap analog) using methods known in the art.

CLDN-18.2 positive: As used herein, the term “CLDN-18.2 positive” or “CLDN-18.2+” refers to clinically relevant CLDN-18.2 expression and/or activity, e.g., as may be associated with a particular disease, disorder, or condition and/or as may be detected in or on a sample that may be or comprise one or more cells or tissue samples. In some embodiments, CLDN-18.2+ refers to cancer that is associated with clinically relevant CLDN-18.2 expression and/activity. In certain exemplary embodiments, CLDN-18.2 positive expression and/or activity may be or comprise de novo CLDN-18.2 overexpression, e.g., in cancer cells; alternatively or additionally, in some embodiments, CLDN-18.2 positive expression and/or activity may be or have been associated with exposure to one or more agents or conditions, such as one or more chemotherapeutic agents (including, e.g., gemcitabine and/or cisplatin). In some embodiments, CLDN-18.2 “positivity” is assessed relative to an appropriate reference (e.g., a “negative control” such as a CLDN-18.2 level and/or activity in appropriately comparable non-cancer cell(s) and/or tissue(s); a “positive control” such as a CLDN-18.2 level and/or activity as may have been determined for known CLDN-18.2-positive cell(s) and/or tissue(s); and/or an established threshold for CLDN-18.2 level and/or activity associated with normal (e.g., healthy, non-cancer) vs non-normal (e.g., cancer) status. In some embodiments, the term “CLDN-18.2+” is used herein to refer to a tumor sample from a cancer patient when that has been determined to show elevated detectable CLDN-18.2 protein expression relative to an appropriate reference (e.g., that level observed in a sample determined or otherwise known to be negative for CLDN-18.2 expression). In some embodiments, a sample is considered to be CLDN-18.2+ when ≥50% of tumor cells in the sample are determined to have ≥2+ CLDN-18.2 protein staining-intensity as assessed by an immunohistochemistry assay in formalin-fixed, paraffin-embedded (FFPE) neoplastic tissues; those skilled in the art are aware that pathologists commonly use such a scoring system for interpretation of IHC data obtained with respect to tumor sample(s). See, e.g., Fedchenko and Reifenrath, Diagnostic Pathology (2014) 9:221, which describes different approaches for interpretation and reporting of IHC analysis results including a scoring system. See also, Zimmermann et al., Cancer Cytopathology (2014) 48-58. Thus, pathologists will readily recognize that 2+ refers to a grading score of 2 or higher, which indicates that such an immunohistochemistry assay result is unambitious. More precisely 2+ describes a moderate or strong staining in a qualitative scale from negative” (0), “weak” (1), “moderate” (2), “strong” (3).

Co-administration: As used herein, the term “co-administration” refers to use of a pharmaceutical composition described herein in combination with another therapy (e.g., surgery, radiation, and/or administration of an another therapeutic agent such as a chemotherapeutic agent described herein, and/or an agent that relieves one or more symptoms or attributes of the relevant disease, disorder or condition and/or of administered therapy [e.g., chemotherapy]), so that a subject receives both. The combined administration of a pharmaceutical composition described herein and such other therapy may be performed concurrently (e.g., via overlapping protocols) or separately (e.g., sequentially in any order). In some embodiments, a pharmaceutical composition described herein may include two or more active agents combined in one pharmaceutically-acceptable carrier (e.g., in a single dosage form). Alternatively, in some embodiments, co-administration involves administration of two or more physically distinct pharmaceutical compositions, each of which may contain a different active agent or combination of agents; in some such embodiments, one or more (and, in some embodiments, all) doses of such distinct pharmaceutical compositions may be administered substantially simultaneously. In some embodiments, one or more (and, in some embodiments, all) doses of such distinct pharmaceutical compositions may be administered separately, e.g., according to overlapping regimens or sequential regimens. In general, two or more therapies may be considered to be “co-administered” when delivered or administered sufficiently close in time that there is at least some temporal overlap in biological effect(s) generated by each on a target cell or a subject to which they are administered.

Combination therapy: As used herein, the term “combination therapy” refers to those situations in which a subject is simultaneously exposed to two or more therapeutic regimens (e.g., two or more therapeutic agents). In some embodiments, two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Contacting: As used interchangeably herein, the term “delivery,” “delivering,” or “contacting” refers to exposing a relevant target (e.g., cell, tissue, organism, etc.) to an ssRNA(s) or a composition that comprises or delivers the same as described herein, so that the ssRNA is delivered into a target cell (e.g., cytosol of a target cell). A target cell can be cultured in vitro or ex vivo or be present in a subject (in vivo). Those skilled in the art will appreciate that different methods of contacting may be utilized to achieve such delivery to a target cell in in vitro, ex vivo, or in vivo applications. In some embodiments, contacting cells in culture may be or comprise in vitro transfection. In some embodiments, contacting may utilize one or more delivery vehicles (e.g., lipid nanoparticles described herein). In some embodiments, contacting may be or comprise administering a pharmaceutical composition described herein to a subject.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Disease: As used herein, the term “disease” refers to a disorder or condition that typically impairs normal functioning of a tissue or system in a subject (e.g., a human subject) and is typically manifested by characteristic signs and/or symptoms. In some embodiments, an exemplary disease is cancer.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an ssRNA (e.g., an mRNA) encodes a polypeptide if transcription and translation of mRNA corresponding to that gene produces the polypeptide in a cell or other biological system. In some embodiments, a coding region of an ssRNA encoding a CLDN-18.2-targeting antibody agent refers to a coding strand, the nucleotide sequence of which is identical to the mRNA sequence of such a CLDN-18.2-targeting antibody agent. In some embodiments, a coding region of an ssRNA encoding a CLDN-18.2-targeting antibody agent refers to a non-coding strand of such a CLDN-18.2-targeting antibody agent, which may be used as a template for transcription of a gene or cDNA.

Epitope: As used herein, the term “epitope” includes any moiety that is specifically recognized by an immunoglobulin (e.g., antibody or receptor) binding component or an aptamer. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an mRNA molecule that begins at the transcription start site and ends one nucleotide (nt) before the start codon (usually AUG) of the coding region of an RNA.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Locally advanced tumor: As used herein, the term “locally advanced tumor” or “locally advanced cancer” refers to its art-recognized meaning, which may vary with different types of cancer. For example, in some embodiments, a locally advanced tumor refers to a tumor that is large but has not yet spread to another body part. In some embodiments, a locally advanced tumor is used to describe cancer that has grown outside the tissue or organ it started but has not yet spread to distant sites in the body of a subject. By way of example only, in some embodiments, locally advanced pancreatic cancer typically refers to stage III disease with tumor extension to adjacent organs (e.g., lymph nodes, liver, duodenum, superior mesenteric artery, and/or celiac trunk) but no signs of metastatic disease; yet complete surgical excision with negative pathologic margins is not possible.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes cancer, or presence of one or more tumors. In some embodiments, a patient is receiving or has received certain therapy to diagnose and/or to treat a disease, disorder, or condition. In some embodiments, a patient is a cancer patient.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Reference/Reference standard: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. In some embodiments, a reference or control is or comprises a set specification (e.g., relevant acceptance criteria). Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety. In some embodiments, a CLDN-18.2-targeting antibody agent encoded by one or more ssRNAs (e.g., ones described herein) does not detectably bind to a competing alternative target (e.g., CLDN18.1 polypeptide) under conditions of binding to a CLDN-18.2 polypeptide. In some embodiments, a CLDN-18.2-targeting antibody agent binds with higher on-rate, lower off-rate, increased affinity, decreased dissociation, and/or increased stability to CLDN-18.2 polypeptide as compared with its competing alternative target(s), including, e.g., CLDN18.1 polypeptide

Specific binding: As used herein, the term “specific binding” refers to an ability to discriminate between possible binding partners in the environment in which binding is to occur. An antibody agent that interacts with one particular target when other potential targets are present is said to “bind specifically” to the target with which it interacts. In some embodiments, specific binding is assessed by detecting or determining degree of association between CDRs of an antibody agent and their partners; in some embodiments, specific binding is assessed by detecting or determining degree of dissociation of an antibody agent-partner complex; in some embodiments, specific binding is assessed by detecting or determining ability of an antibody agent to compete an alternative interaction between its partner and another entity. In some embodiments, specific binding is assessed by performing such detections or determinations across a range of concentrations.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., cancer). In some embodiments, a subject is susceptible to a disease, disorder, or condition (e.g., cancer). In some embodiments, a subject displays one or more symptoms or characteristics of a disease, disorder, or condition (e.g., cancer). In some embodiments, a subject displays one or more non-specific symptoms of a disease, disorder, or condition (e.g., cancer). In some embodiments, a subject does not display any symptom or characteristic of a disease, disorder, or condition (e.g., cancer). In some embodiments, a subject is someone with one or more features characteristic of susceptibility to or risk of a disease, disorder, or condition (e.g., cancer). In some embodiments, a subject is a patient. In some embodiments, a subject is an individual to whom diagnosis and/or therapy is and/or has been administered.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition; carrier of a genetic marker or other biomarker associated with the disease, disorder or condition, etc.).

Suffering from: An individual who is “suffering from” a disease, disorder, and/or condition has been diagnosed with and/or displays one or more symptoms of a disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapeutic agent: As used interchangeably herein, the phrase “therapeutic agent” or “therapy” refers to an agent or intervention that, when administered to a subject or a patient, has a therapeutic effect and/or elicits a desired biological and/or pharmacological effect. In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to the sequence of an mRNA molecule that begins following the stop codon of the coding region of an open reading frame sequence. In some embodiments, the 3′ UTR begins immediately after the stop codon of the coding region of an open reading frame sequence. In other embodiments, the 3′ UTR does not begin immediately after stop codon of the coding region of an open reading frame sequence

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Unresectable tumor: As used herein, the term “unresectable tumor” typically refers to a tumor characterized by one or more features that, in accordance with sound medical judgement, are considered to indicate that the tumor cannot safely (e.g., without undue harm to the subject) be removed by surgery, and/or with respect to which a competent medical profession has determined that risk to the subject of tumor removal outweighs benefits associated with such removal. In some embodiments, an unresectable tumor refers to a tumor that involves and/or has grown into an essential organ or tissue (including blood vessels that may not be reconstructable) and/or that is otherwise in a location that cannot readily be surgically accessed without unreasonable risk of damage to one or more other critical or essential organs and/or tissues (including blood vessels). In some embodiments, “unresectability” of a tumor refers to the likelihood of achieving a margin-negative (RO) resection. In the context of pancreatic cancer, encasement of major vessels by a tumor such as superior mesenteric artery (SMA) or celiac axis, portal vein occlusion, and the presence of celiac or para-aortic lymphadenopathy are generally acknowledged as findings that preclude RO surgery. Those skilled in the art will understand parameters that determine whether a tumor is unresectable or not.

Those skilled in the art, reading the present specification, will appreciate that, in many embodiments, standard techniques are available and may be used for recombinant DNA, oligonucleotide synthesis, tissue culture and/or transformation (e.g., electroporation, lipofection, transfection). Enzymatic reactions and/or purification techniques may typically be performed according to manufacturer's specifications or as commonly accomplished in the art or as described herein. In many embodiments, foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

Outcomes of Standard of Care (SOC) therapy remain poor for many cancer patients, and particularly for those with relapsed or refractory advanced solid tumors. Treatment options typically include further palliative chemotherapy, which might be less tolerated after previous repeated exposure to cytotoxic compounds, or best supportive care, and investigational treatments without proven benefit. Therapy in this population is not curative, with an expected overall survival of a few months. Immunotherapy has emerged as an effective treatment option in some cancers with high unmet medical need. Specifically, immune checkpoint inhibitors are approved for treatment across various cancer indications and act by invigorating pre-existent anti-tumor-specific T cells. The medical need is still high for various cancer types. The present disclosure, among other things, provides insights and technologies for treating cancer (e.g., pancreatic cancer and/or biliary cancer) with a therapy targeting Claudin-18.2 (CLDN-18.2).

In some embodiments, the present disclosure, among other things, provides RNA technologies to deliver a monoclonal antibody targeting CLDN-18.2 that combines both potent anti-tumoral features and an excellent safety profile, skipping the hurdle of slow and cumbersome antibody manufacturing process. Without wishing to be bound by any particular theory, the present disclosure proposes that such RNA delivering modality may achieve one or more improvements such as effective administration with reduced incidence (e.g., frequency and/or severity) of treatment emergent adverse events (“TEAEs”), and/or with improved relationship between efficacy level and TEAE level (e.g., improved therapeutic window) relative to those observed when a corresponding (e.g., encoded) protein (e.g., antibody) agent itself is administered. In particular, the present disclosure teaches that such improvements in particular may be achieved by delivering IMAB362 via administration of a nucleic acid, and in particular of RNA(s) (e.g., ssRNA(s) such as mRNA(s))) encoding it.

In some embodiments, the present disclosure, among other things, provides insights that mRNA(s) encoding an antibody agent (e.g., IMAB362) or a functional portion thereof, optionally formulated with lipid nanoparticles (LNP) for intravenous (IV) administration to a subject (e.g., a human patient, a model organism, etc.), can be taken up by target cells (e.g., liver cells) for efficient production of the encoded antibody agent (e.g., IMAB362) at therapeutically relevant plasma concentrations, for example, as illustrated in FIG. 14 for a CLDN-18.2-targeting antibody agent expressed from ssRNAs (e.g., ones described herein). In some embodiments, antibody agents are expressed from mRNA, e.g., engineered for minimal immunogenicity, and/or formulated in lipid nanoparticles (LNPs). In some embodiments, mRNA that encodes an antibody agent may comprise modified nucleotides (e.g., but not limited to pseudouridine).

Moreover, the present disclosure, among other things, provides an insight that the capability of a CLDN-18.2-targeting antibody agent delivered as described herein can induce antibody-dependent cellular cytotoxicity (ADCC) and/or complement-dependent cytotoxicity (CDC) against target cells (e.g., tumor cells) while leveraging immune system of recipient subjects can augment cytotoxic effect(s) of chemotherapy and/or other anti-cancer therapy. In some embodiments, such a combination therapy may prolong progression-free and/or overall survival, e.g., relative to the individual therapies administered alone and/or to another appropriate reference.

Without wishing to be bound by a particular theory, the present disclosure observes that certain chemotherapeutic agents, for example such as gemcitabine, oxaliplatin, and 5-fluorouracil were shown to upregulate existing CLDN-18.2 expression levels in pancreatic cancer cell lines; moreover, these agents were not observed to increase de novo expression in CLDN-18.2-negative cell lines. See, for example, Tureci et al., (2019) “Characterization of Zolbetuximab in pancreatic cancer models.” In Oncoimmunology 8 (1), pp. e1523096.

The present disclosure, among other things, provides an insight that CLDN-18.2-targeted therapy as described herein may be particularly useful and/or effective when administered to tumor(s) (e.g., tumor cells, subjects in whom such tumor(s) and/or tumor cell(s) are suspected and/or have been detected, etc.) characterized by (e.g., that have been determined to display and/or that are expected or predicted to display) elevated expression and/or activity of CLDN-18.2 expression in tumor cells (e.g., as may result or have resulted from exposure to one or more chemotherapeutic agents). Indeed, among other things, the present disclosure teaches that provided CLDN-18.2-targeted therapy (e.g., administration of a nucleic acid such as an RNA and, more particularly an mRNA encoding a CLDN-18.2-targeting antibody agent) as described herein may provide synergistic therapeutic when administered in combination with (e.g., to a subject who has received and/or is receiving or has otherwise been exposed to) one or more CDLN-18.2-enhancing agents (e.g., one or more certain chemotherapeutic agents). Accordingly, in some embodiments, CLDN-18.2-targeted therapy as described herein can be useful in combination with other anti-cancer agents that are expected to and/or have been demonstrated to up-regulate CLDN-18.2 expression and/or activity in tumor cells.

Accordingly, the present disclosure, among other things, provides insights and technologies for treating cancer, particularly, cancers that are associated with expression of CLDN-18.2. In some embodiments, provided technologies are effective for treatment of pancreatic cancers. In some embodiments, provided technologies are effective for treatment of gastric or gastro-esophageal cancers. In some embodiments, provided technologies are effective for treatment of biliary cancers. In some embodiments, provided technologies are effective for treatment of ovarian cancers. In some embodiments, provided technologies are effective when applied to locally advanced tumors. In some embodiments, provided technologies are effective when applied to unresectable tumors. In some embodiments, provided technologies are effective when applied to metastatic tumors.

### I. Claudin-18.2 Polypeptide

Claudin-18.2 (CLDN-18.2) is a cancer-associated splice variant of Claudin-18. CLDN-18.2 is a member of the Claudin family of more than 20 structurally related proteins that are involved in the formation of tight junctions in epithelia and endothelia.

CLDN18 expression in healthy tissues. Claudin18.2 is a 27.8 kDa protein with four membrane-spanning domains and two small extracellular loops (Niimi et al. 2001). CLDN-18.2 is a tight junction molecule of the gastric epithelia. Gastric tight junctions are highly specialized on repelling gastric acid, which may injure the gastric lining.

CLDN-18.2 is a highly selective gastric lineage antigen (Sahin et al. 2008). Typically, its expression is restricted to short-lived differentiated cells of gastric epithelia in the pit and base regions of gastric glands. The stem cell zone, from which differentiated epithelial cells of the gastric glands are continuously replenished, is CLDN-18.2-negative. Without wishing to be bound by theory, it is commonly believed that no other normal cell type of the human body expresses CLDN-18.2 at transcript level or at protein level.

CLDN18 expression in cancer. CLDN-18.2 is expressed in various human cancers including gastric, gastroesophageal (GE) and pancreatic cancers (PC) (Karanjawala et al. 2008; Coati et al. 2019) and precancerous lesions (Woll et al. 2014; Tanaka et al. 2011). Tumor-associated expression of CLDN-18.2 has also been detected in ovarian (Sahin et al. 2008), biliary (Shinozaki et al. 2011) and lung cancers (Micke et al. 2014).

About 77% of primary gastric adenocarcinomas (GAC) are CLDN-18.2+. 56% of GAC display strong CLDN-18.2 expression defined as staining intensity ≥2+ by immunohistochemical analysis in at least 60% of tumor cells. CLDN-18.2 expression is more frequent in diffuse than in intestinal gastric cancers. The CLDN-18.2 protein is also frequently detected in lymph node metastases of gastric cancer and in distant metastases into the ovaries (so-called Krukenberg tumors). Moreover, 50% of esophageal adenocarcinomas display significant expression of CLDN-18.2.

In pancreatic cancer, CLDN-18.2 is expressed with a prevalence of 60-90% in pancreatic ductal adenocarcinoma (PDAC) (Karanjawala et al. 2008; Wöll et al. 2014). PDAC, accounting for over 80% of all pancreatic neoplasms, is the seventh most frequent cancer in Europe and fourth of cancer-related causes of death in the European Union (Ferlay et al. 2010; Jemal et al. 2011; Seufferlein et al. 2012). Almost 60% of patients with PDAC express membrane-bound CLDN-18.2 and in 20% of patients with pancreatic neuroendocrine neoplasms CLDN-18.2 is ectopically activated. CLDN-18.2 is expressed in primary and metastatic PDAC lesions (Wöll et al. 2014).

Down-regulation of CLDN-18.2 by siRNA technology has shown to result in inhibition of proliferation of gastric cancer cells (Niimi et al. 2001), indicating an involvement in proliferation of CLDN-18.2+ tumor cells.

### II. Exemplary Antibody Agents Targeting Claudin-18.2 Polypeptides

In some embodiments, an antibody agent targeting CLDN-18.2 specifically binds to a CLDN-18.2 polypeptide. In some embodiments, an antibody agent targeting CLDN-18.2 specifically binds to a first extracellular domain (ECD1) of a CLDN-18.2 polypeptide. For example, in some embodiments, such an antibody agent specifically binds to an epitope of ECD1 that is exposed in cancer cells. In some embodiments, such an antibody agent may have a binding affinity (e.g., as measured by a dissociation constant) for a CLDN-18.2 polypeptide, e.g., an epitope of ECD1 of a CLDN-18.2 polypeptide) of at least about 10-4 M, at least about 10-5 M, at least about 10⁻⁶ M, at least about 10⁻⁷ M, at least about 10⁻⁸ M, at least about 10⁻⁹ M, or lower. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, gel-shift assays, pull-down assays, equilibrium dialysis, analytical ultracentrifugation, surface plasmon resonance (SPR), bio-layer interferometry, grating-coupled interferometry, and spectroscopic assays.

In some embodiments, an antibody targeting CLDN-18.2 may bind specifically to a CLDN-18.2 polypeptide relative to a CLDN18.1 polypeptide. In some embodiments, an antibody targeting CLDN-18.2 does not bind to any other claudin family member including the closely related splice variant 1 of Claudin-18 (CLDN18.1) that is predominantly express in tissues, e.g., lung.

In some embodiments, an antibody agent targeting CLDN-18.2 may be any one of CLDN-18.2-targeting antibodies described in WO 2007/059997, WO2008/145338, and WO2013/174510, the contents of each of which are incorporated herein by reference in their entirety for the purposes described herein.

In some embodiments, an antibody agent targeting CLDN-18.2 comprises (a) a variable heavy chain domain having at least one CDR (including, e.g., 1 CDR, 2 CDRs, and 3 CDRs) selected from the group consisting of: (i) CDR1 represented by amino acid residues (GYTFTSYW); (ii) CDR2 represented by amino acid residues (IYPSDSYT); and (iii) CDR3 represented by amino acid residues (TRSWRGNSFDY); and/or (b) a variable light chain domain having at least one CDR (including, e.g., 1 CDR, 2 CDRs, and 3 CDRs) selected from the group consisting of (i) CDR1 represented by amino acid residues (QSLLNSGNQKNY); (ii) CDR2 represented by amino acid residues (WAS); and (iii) CDR3 represented by amino acid residues (QNDYSYPFT).

In some embodiments, an antibody agent targeting CLDN-18.2 has a heavy chain amino acid sequence and a light chain amino acid sequence, that is or includes relevant sequences (e.g., variable region sequences, e.g., CDR and/or framework (FR) sequences) as described in U.S. Pat. No. 9,751,934. For example, in some embodiments, an antibody agent targeting CLDN-18.2 has a heavy chain consisting of or comprising an amino acid sequence represented by amino acid residues 20-467 of SEQ ID NO: 1 as set forth below (wherein SEQ ID NO: 1 here corresponds to SEQ ID NO: 118 of U.S. Pat. No. 9,751,934 and the underlined amino acid sequence of SEQ ID NO: 1 corresponds to a secretion signal sequence), and a light chain consisting of or comprising an amino acid represented by amino acid residues 21-240 of SEQ ID NO: 2 as set forth below (wherein SEQ ID NO: 2 here corresponds to SEQ ID NO: 125 of U.S. Pat. No. 9,751,934 and the underlined amino acid sequence of SEQ ID NO: 2 corresponds to a secretion signal sequence).

(SEQ ID NO: 1)
MGWSCIILFLVATATGVHSQVQLQQPGAELVRPGASVKLSCKASGYTFTS
YWINWVKQRPGQGLEWIGNIYPSDSYTNYNQKFKDKATLTVDKSSSTAYM
QLSSPTSEDSAVYYCTRSWRGNSFDYWGQGTTLTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK
(SEQ ID NO: 2)
MESQTQVLMSLLFWVSGTCGDIVMTQSPSSLTVTAGEKVTMSCKSSQSLL
NSGNQKNYLTWYQQKPGQPPKLLIYWASTRESGVPDRFTGSGSGTDFTLT
ISSVQAEDLAVYYCQNDYSYPFTFGSGTKLEIKRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGEC

In some embodiments, an antibody agent targeting CLDN-18.2 comprises (a) a variable heavy chain domain having at least one CDR (including, e.g., 1 CDR, 2 CDRs, and 3 CDRs) selected from the group consisting of: (i) CDR1 represented by amino acid residues 45-52 of SEQ ID NO: 1; (ii) CDR2 represented by amino acid residues 70-77 of SEQ ID NO: 1; and (iii) CDR3 represented by amino acid residues 116-126 of SEQ ID NO: 1; and/or (b) a variable light chain domain having at least one CDR (including, e.g., 1 CDR, 2 CDRs, and 3 CDRs) selected from the group consisting of (i) CDR1 represented by amino acid residues 47-58 of SEQ ID NO: 2; (ii) CDR2 represented by amino acid residues 76-78 of SEQ ID NO: 2; and (iii) CDR3 represented by amino acid residues 115-123 of SEQ ID NO: 2.

In some embodiments, an antibody agent targeting CLDN-18.2 has a heavy chain consisting of or comprising the amino acid sequence of SEQ ID NO: 1 and a light chain consisting of or comprising the amino acid sequence of SEQ ID NO: 2.

In some embodiments, an antibody agent targeting CLDN-18.2 can be engineered to decrease potential immunogenicity and/or improve secretion. For example, in some embodiments, a murine secretion signal sequence of an antibody agent targeting CLDN-18.2 can be replaced by a human one.

In some embodiments, an antibody agent targeting CLDN-18.2 has a heavy chain consisting of or comprising an amino acid sequence represented by amino acid residues 27-474 of SEQ ID NO: 3 as set forth below (wherein the underlined amino acid sequence corresponds to a secretion signal sequence); and a light chain consisting of or comprising an amino acid represented by amino acid residues 27-246 of SEQ ID NO: 4 as set forth below (wherein the underlined amino acid sequence corresponds to a secretion signal sequence).

(SEQ ID NO: 3)
MRVMAPRTLILLLSGALALTETWAGSQVQLQQPGAELVRPGASVKLSCKA
SGYTFTSYWINWVKQRPGQGLEWIGNIYPSDSYTNYNQKFKDKATLTVDK
SSSTAYMQLSSPTSEDSAVYYCTRSWRGNSFDYWGQGTTLTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGK
(SEQ ID NO: 4)
MRVMAPRTLILLLSGALALTETWAGSDIVMTQSPSSLTVTAGEKVTMSCK
SSQSLLNSGNQKNYLTWYQQKPGQPPKLLIYWASTRESGVPDRFTGSGSG
TDFTLTISSVQAEDLAVYYCQNDYSYPFTFGSGTKLEIKRTVAAPSVFIF
PPSDEQLKSGTASVVCLLNNFYPREAKVQWKVDNALQSGNSQESVTEQDS
KDSTYSLSSTLTLSKADYEKHKVYACEVTHQGLSSPVTKS

In some embodiments, an antibody agent targeting CLDN-18.2 has a heavy chain consisting of or comprising the amino acid sequence of SEQ ID NO: 3 and a light chain consisting of or comprising the amino acid sequence of SEQ ID NO: 4.

In some embodiments, an antibody targeting CLDN-18.2 is IMAB362 (also known as Zolbetuximab, Claudiximab). IMAB362, an antibody targeting CLDN-18.2, is in advanced clinical development (NCT01630083, NCT03816163, NCT03653507, NCT03505320, NCT03504397) and known in the art (see, e.g., Sahin et al. 2018; Sahin et al. 2017; Al-Batran et al. 2017a; Al-Batran et al. 2017b; TUreci et al. 2019; Trarbach et al. 2014; Morlock et al. 2018a; Schuler et al. 2016; Lordick et al. 2016; Morlock et al. 2018b). Its target CLDN-18.2 is a highly selective tumor-associated surface marker.

IMAB362, developed by Ganymed Pharmaceuticals GmbH and acquired by Astellas Pharma Inc., is a full IgG1 antibody targeting the tight junction protein CLDN-18.2 and mediates cell death through antibody-dependent cellular cytotoxicity (ADCC) and complement-dependent cytotoxicity (CDC). IMAB362 recognizes the first extracellular domain (ECD1) of CLDN-18.2 with high affinity and specificity (Sahin et al. 2008; Tureci et al. 2011). The epitope is not accessible in normal epithelial barriers to the antibody. Disruption of tight junctions and loss of cell polarization are early hallmarks of cancer. In this process, the epitope of IMAB362 is exposed. IMAB362 does not bind to any other claudin family member including the closely related splice variant 1 of Claudin 18 (CLDN18.1) that is predominantly expressed in tissues, e.g., lung.

IMAB362 plus epirubicin, oxaliplatin, and capecitabine (EOX) were tested in phase 2 FAST trial (NCT01630083) against EOX in first-line patients with gastric and gastro-esophageal cancer (Morlock et al. 2018a; Schuler et al. 2016; Al-Batran et al. 2016; Lordick et al. 2016; Morlock et al. 2018b). The FAST patient population included patients whose tumors had ≥40% of tumor cells expressing CLDN-18.2 with a moderate-to-strong (≥2+) staining intensity. The subset of patients whose tumors had ≥70% of tumor cells with ≥2+ CLDN-18.2 staining intensity derived the greatest benefit from IMAB362 treatment at the 800/600 mg/kg 2 dose with near-doubling of their median overall survival (OS) (Al-Batran et al. 2016; Lordick et al. 2016). The benefit of IMAB362 in OS in the ≥70% CLDN-18.2 expression (+33.1 weeks; p<0.0005) was accompanied by a significant delay in central independent reviewed progression (+14.5 weeks; p<0.0005) and a higher objective response rate (ORR) (35.1% vs 27.1%). Addition of IMAB362 to EOX did not negatively impact patient-related outcome. No significant differences between the treatment arms were observed in the Mixed effect Model Repeat Measurement for global health state or total ST022 score throughout the study, but IMAB362 plus EOX significantly delayed deterioration of the global health score by 2.6 months vs EOX alone (p=0.008).

IMAB362 is also tested by Astellas Pharma Inc. in a global development program in Phase 2 and 3 trials in patients with CLDN-18.2+ gastric/gastroesophageal and pancreatic cancer.

IMAB362 has been tested in various clinical trials as shown in Table 1 below.

TABLE 1
Summary of certain clinical trials involving administration of IMAB362

| Phase | | | |
|---|---|---|---|
| Identifier | | | |
| Name | Patient population | Treatment | Ref. |
| Ph1 | CLDN-18.2+ gastroesophageal/gastric | IMAB362, single | (Sahin et al. 2018; |
| NCT00909025 | adenocarcinoma; | agent, single dose | Sahin et al. 2017) |
| FIH trial | inoperable locally advanced refractory | | |
| | or recurrent to standard therapy | | |
| Ph1/Ph2a | CLDN-18.2+ gastric, | IMAB362, | (Al-Batran et al. 2017b) |
| NCT01671774 | gastroesophageal/GEJ carcinoma | repeated | |
| PILOT trial | | dose + IL-2 + | |
| | | Zoledronic acid | |
| Ph2a | CLDN-18.2+ gastric, | IMAB362 | (Tureci et al. 2019; |
| NCT01197885 | gastroesophageal/GEJ adenocarcinoma; | single agent, | Trarbach et al. 2014; |
| MONO trial | metastatic, refractory or recurrent | repeated dose | Morlock et al. 2018a) |
| Ph2 RCT 3-arm | CLDN-18.2+ gastric, | IMAB362, | (Morlock et al. 2018a; |
| NCT01630083 | gastroesophageal/GEJ adenocarcinoma; | repeated dose. + | Schuler et al. 2016; |
| FAST trial | inoperable locally advanced, resections | EOX versus EOX | Al-Batran et al. 2016; |
| | with R2 outcome or metastatic disease - | alone (epirubicin, | Lordick et al. 2016; |
| | 1st line | oxaliplatin, | Morlock et al. 2018b) |
| | | capecitabine) | |
| Ph1 | Gastric or Gastro-esophageal Junction | IMAB362 single | |
| NCT04086758 | (GEJ) Adenocarcinoma | agent | |
| Ph1 | Advanced or metastatic CLDN-18.2+ | IMAB362 single | |
| NCT03528629 | Gastric and Gastro-esophageal Junction | agent | |
| | (GEJ) Cancer | | |
| Ph2 | Metastatic pancreatic cancer/ | IMAB362 + | |
| NCT03816163 | adenocarcinoma | nab-paclitaxel + | |
| | | gemcitabine | |
| Ph2 | CLDN-18.2 Positive, Metastatic or | IMAB362 single | |
| NCT03505320 | Advanced Unresectable Gastric and | agent vs | |
| ILUSTRO | Gastroesophageal Junction (GEJ) | IMAB362 + | |
| trial | Adenocarcinoma | mFOLFOX6 | |
| Ph3 RCT | Locally advanced unresectable or | IMAB362 + | |
| NCT03653507 | metastatic Gastric or GEJ, | CAPOX | |
| GLOW trial | Adenocarcinoma or Cancer; | (Capecitabine + | |
| | CLDN-18.2+, HER2-negative, | Oxaliplatin); | |
| | 1^{st} line treatment | Placebo + CAPOX | |
| Ph3 | Locally advanced unresectable or | IMAB362 + | |
| NCT03504397 | metastatic gastric or GEJ; | mFOLFOX6; | |
| SPOTLIGHT | Adenocarcinoma or Cancer; | Placebo + | |
| trial | CLDN-18.2+, HER2-negative | mFOLFOX6 | |

The safety profile of IMAB362 in patients is well characterized and repeated doses up to 1000 mg/m² q3w (cₘₐₓ of up to 603 μg/mL) have been tolerated without dose limiting toxicities (Sahin et al. 2018; TUreci et al. 2019).

Without wishing to be bound by a particular theory, a main pharmacological mode of action of IMAB362 for executing tumor cell killing involves antibody-dependent cellular cytotoxicity (ADCC). Based on dose-response curves obtained by in vitro ADCC testing the concentration of a drug that gives 95% response is observed at IMAB362 concentrations of 0.3-28 μg/mL in serum (Sahin et al. 2018). For example, efficient lysis of CLDN-18.2+ cells through ADCC with an EC₉₅ of 0.3-28 μg/mL has been reported (Sahin et al. 2018).

Across various trials, IMAB362 was well tolerated, with nausea and vomiting being the dominant adverse events (AE), with no observed dose limiting toxicity (DLT) and clinical activity as a single agent and in combination with chemotherapy.

Among other things, the present disclosure provides an insight that IMAB362 or a variant thereof (e.g., a variant that shares one or more features of IMAB362, including, e.g., one or more (and in many embodiments all) CDR sequences, one or more (and in many embodiments all) FR sequences, and/or heavy and/or light chain variable sequences, etc., and/or that is a class variant such as IgG1, IgM, IgA, etc.) may represent a particularly desirable antibody for delivery via administration of a ribonucleic acid as described herein. Without wishing to be bound by any particular theory, the present disclosure proposes that such delivering modality may achieve effective administration with reduced incidence (e.g., frequency and/or severity) of IMAB362 treatment-related adverse events (TEAEs) relative to those observed when IMAB362 antibody itself is administered. In the Phase 2a MONO trial with IMAB362 (NCT01197885), TEAEs occurred in 82% (n=44/54) of the patients; nausea (61%), vomiting (50%) and fatigue (22%) were the most frequent TEAEs. Grade 3 vomiting was reported in 12 patients (22%) and grade 3 nausea in eight patients (15%). These patients received the 600 mg/m² dose. The nausea and vomiting observed in this study were managed by pausing or slowing infusion of IMAB362 indicating that the AEs are Cₘₐₓ related (Türeci et al. 2019).

In particular, the present disclosure, among other things, demonstrates that the pharmacokinetic (PK) profile of IMAB362 delivered as a ribonucleic acid (“RiboMab01”) described herein showed a gradual increase in antibody concentrations and a notably lower Cₘₐₓ than IMAB362 between 48-72 hours post administration. The altered PK profile of RiboMab01 may reduce the Cₘₐₓ-related AEs seen in patients after treatment with IMAB362. The present disclosure also provides non-human primate study data, which shows that no systemic side effects such as diarrhea were observed.

Among other things, the present disclosure appreciates the favorable risk/benefit profile observed for administered IMB362 antibody, particularly in certain indications with high medical need, and proposes that delivery as described herein may be effective and/or particularly desirable.

### III. RNA Technologies for Delivery of Antibody-Based Therapeutics

Recombinant protein antibodies are widely used biologics for the treatment of diseases or disorders (e.g., cancer) but show a number of limitations, including, e.g., lengthy manufacturing process development and, for antibody derivatives, short serum half-life. The present disclosure, among other things, provides technologies that address certain limitations of recombinant antibody technologies, including for example, lengthy manufacturing process development, and for antibody derivatives, short serum half-life, by utilizing RNA technologies as a modality to express antibody agents, called RiboMabs, directly in the patient's cells as a novel class of antibody-based therapeutics. In some embodiments, the present disclosure, among other things, provides insights that RiboMabs that are formulated with lipid nanoparticles (LNP) for intravenous (IV) administration can be taken up by cells (e.g., liver cells) for efficient production of the encoded RiboMab antibody at therapeutically relevant plasma concentrations (FIG. 14). In some embodiments, RiboMabs are antibody agents encoded by mRNA, e.g., engineered for minimal immunogenicity, and/or formulated in lipid nanoparticles (LNPs). In some embodiments, mRNA that encodes an antibody agent may comprise modified nucleotides (e.g., but not limited to pseudouridine).

RiboMab technology can be utilized to deliver various antibody formats. For example, in some embodiments, RiboMab technology can be used to express a full immunoglobulin (Ig), including, e.g., but not limited to IgG. In some embodiments, a full immunoglobulin (Ig) may be encoded by a single ssRNA comprising a first coding region that encodes a heavy chain of an antibody and a second coding region that encodes a light chain variable domain of the antibody, wherein the single ssRNA comprises or encodes either an internal ribosome entry sides (IRES) or another internal promoter or peptide sequence such as “self-cleaving” 2A or 2A-like sequences (see, e.g., Szymczak et al. Nat Biotechnol 22:589, May 2004; ePub Apr. 4, 2004) to yield a respective heavy chain and light chain, which can then be processed to form a full IgG. In some embodiments, a full Ig may be encoded by two separate ssRNAs: a first ssRNA comprising a coding region that encodes a heavy chain of an antibody; and a second ssRNA comprising a coding region that encodes a light chain of the antibody. Such first and second ssRNAs are then translated into respective chains of an antibody and form a full Ig antibody in target cells.

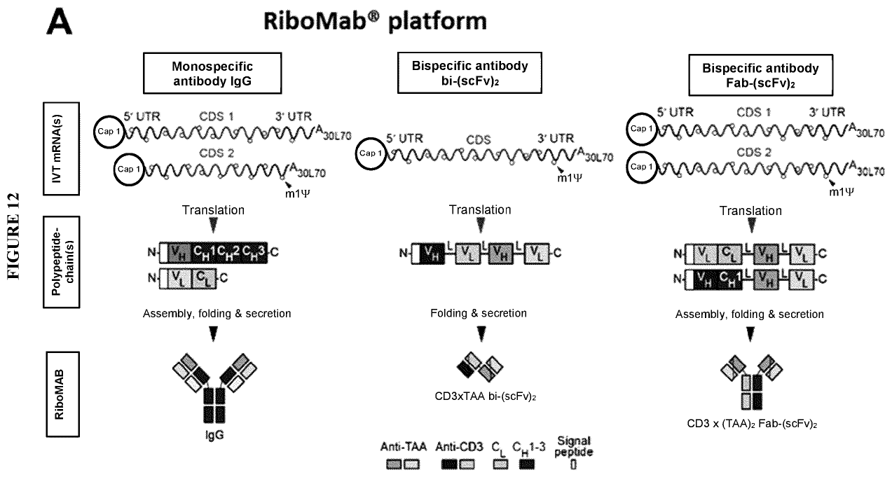

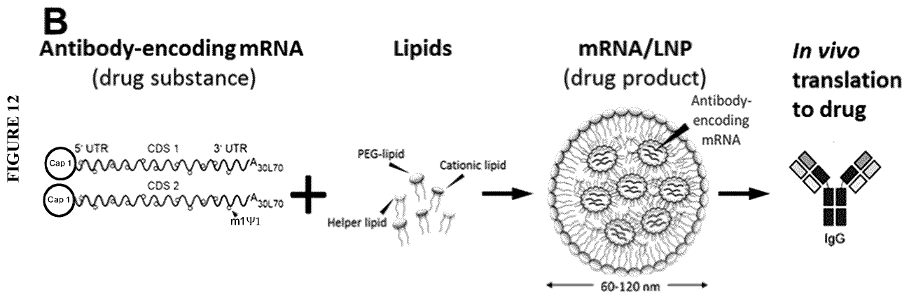

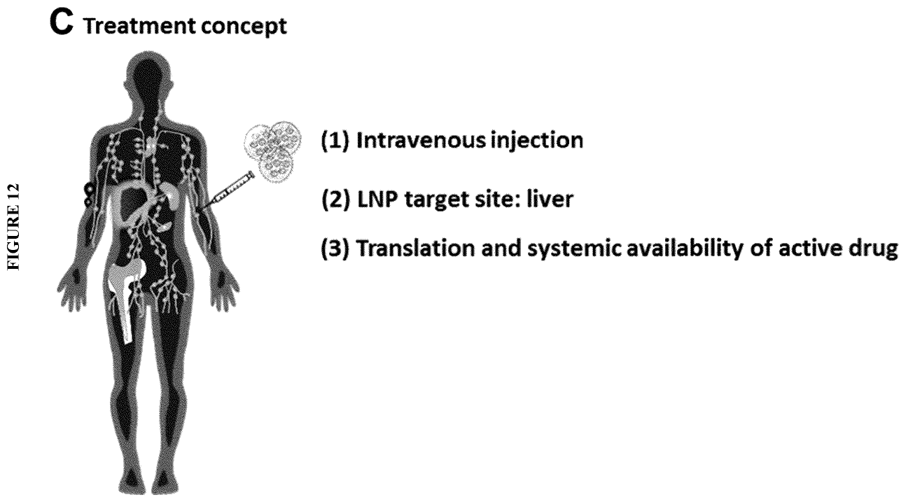

In some embodiments, RiboMab technology can be used to express a bispecific antibody variant, e.g., as illustrated in FIG. 12 (Panel A) or described in Stadler et al. (2016) Oncoimmunology 5(3): e1091555; and/or in Stadler et al. (2017) Nature Medicine 23(7): 815-817. For example, in some embodiments, a bivalent antibody agent may be encoded by a single ssRNA comprising a first coding region that encodes a single-chain variable fragment (scFv) for a first target and a second coding region that encodes a scFv for a second target. In some embodiments, a bivalent antibody agent may be encoded by two separate ssRNAs: a first ssRNA comprising a coding region that encodes a scFv for a first target and a coding region that encodes a heavy chain antigen binding fragment (Fab) for a second target; and a second ssRNA comprising a coding region that encodes a scFv for the same first target and a coding region that encodes a light chain Fab for the same second target. Such first and second ssRNAs are then translated into subunits of an antibody and form a bispecific antibody in target cells.

In some embodiments, RNA agents (e.g., ssRNAs described herein) may be delivered with a carrier. In some embodiments, RNA/LNP is intravenously (IV) administered and taken up by target cells (e.g., liver cells) for efficient production of the encoded RiboMab antibody at therapeutically relevant plasma concentrations.

A. Provided Single-Stranded RNAs (ssRNAs) Encoding Antibody Agents Directed to Claudin-18.2 Polypeptides and Compositions Thereof

In some embodiments, at least one single-stranded RNA (ssRNA) comprises one or more coding regions that encode an antibody agent as described in the section entitled “Exemplary antibody agents targeting Claudin-18.2 polypeptides” above. In some embodiments, at least one ssRNA comprises one or more coding regions that encode an antibody agent IMAB362 as described above or exemplified herein.

Without wishing to be bound by any particular theory, the present disclosure, among other things, provides an insight that, in some embodiments, an antibody agent IMAB362 may be particularly useful and/or effective at least in part because it binds specifically to CLDN-18.2 and, moreover, binds preferentially to CLDN-18.2 relative to CLDN18.1. In some embodiments, teachings provided herein may be applicable to other antibody agents specific to CLDN-18.2, and in particular to such antibodies that bind preferentially to CLDN-18.2 even relative to CLDN18.1. For example, in some embodiments, at least one single-stranded RNA (ssRNA) comprises one or more coding regions that encode an antibody agent that binds preferentially to a CLDN-18.2 polypeptide relative to a CLDN18.1 polypeptide. In some embodiments, such an antibody agent has a binding affinity for a CLDN-18.2 polypeptide higher than that for a CLDN18.1 polypeptide by at least 50% or more including, e.g., at least 60%, at least 70%, at least 80%, at least 90%, at least 95% or higher. In some embodiments, such an antibody agent has a binding affinity for a CLDN-18.2 polypeptide higher than that for a CLDN18.1 polypeptide by at least 1.1-fold or more including, e.g., at least 2-fold, at least 5-fold, at least 10-fold, at least 25-fold, at least 50-fold, at least 75-fold, at least 100-fold, at least 500-fold, at least 1000-fold, at least 5000-fold, at least 10,000-fold or higher. In some embodiments, such an antibody agent does not detectably bind to any other claudin family member including CLDN18.1. In some embodiments, an antibody agent may be or comprise an antibody. In some embodiments, an antibody agent may be or comprise an antigen binding fragment.

In some embodiments, an antibody agent that targets CLDN-18.2 (and may be encoded by an RNA such as an ssRNA, e.g., an mRNA as described herein) specifically binds to a first extracellular domain (ECD1) of a CLDN-18.2 polypeptide. For example, in some embodiments, such an antibody agent specifically binds to an epitope of ECD1 that is exposed in cancer cells.

In some embodiments, at least one ssRNA encodes a variable heavy chain (V_{H}) domain of a CLDN-18.2-targeting antibody agent and a variable light chain (V_{L}) domain of the antibody agent. In some embodiments, such V_{H} domain(s) and V_{L} domain(s) of a CLDN-18.2-targeting antibody agent may be encoded by a single ssRNA construct; alternatively in some embodiments they may be encoded separately by at least two individual ssRNA constructs. For example, in some embodiments, an ssRNA as utilized herein comprises two or more coding regions, which comprises a heavy chain-coding region that encodes at least a V_{H} domain of a CLDN-18.2-targeting antibody agent; and a light chain-coding region that encodes at least a V_{L} domain of a CLDN-18.2-targeting antibody agent. In alternative embodiments, a composition comprises (i) a first ssRNA comprising a heavy chain-coding region that encodes at least a V_{H} domain of a CLDN-18.2-targeting antibody agent; and (ii) a second ssRNA comprising a light chain-coding region that encodes at least a V_{L} domain of a CLDN-18.2-targeting antibody agent.

In some embodiments, a heavy chain-coding region can further encode a constant heavy chain (C_{H}) domain; and/or a light chain-coding region can further encode a constant light chain (C_{L}) domain. For example, in some embodiments, a heavy chain-coding region may encode a V_{H} domain, a C_{H1} domain, a C_{H2} domain, and a C_{H3} domain of a CLDN-18.2-targeting antibody agent in an immunoglobulin form (e.g., IgG); and/or a light chain-coding region may encode a V_{L} domain and a C_{L} domain of a CLDN-18.2-targeting antibody agent in an Ig form (e.g., IgG). For example, in some embodiments, a full immunoglobulin (Ig) may be encoded by a single ssRNA comprising a first coding region that encodes a heavy chain of a CLDN-18.2 Ig antibody (e.g., IgG) and a second coding region that encodes a light chain variable domain of the CLDN-18.2 Ig antibody (e.g., IgG), which single ssRNA requires protein translation to yield a fusion protein comprising a heavy chain and a light chain of the antibody and post-translational cleavage of the fusion protein by a suitable protease into respective heavy chain and light chain, which can then be processed to form a full Ig (e.g., IgG). In some embodiments, a full Ig may be encoded by two separate ssRNAs: a first ssRNA comprising a coding region that encodes a heavy chain of a CLDN-18.2 Ig antibody (e.g., IgG); and a second ssRNA comprising a coding region that encodes a light chain of the CLDN-18.2 Ig antibody (e.g., IgG). Such first and second ssRNAs are then translated into respective chains of an antibody and form a full Ig antibody (e.g., IgG) in target cells. In some embodiments, an antibody agent encoded by one or more ssRNAs in an IgG form is IgG1.

In some embodiments, a heavy chain-coding region of an ssRNA consists of or comprises a nucleotide sequence that encodes at least one CDR (including, e.g., 1 CDR, 2 CDRs, and 3 CDRs) selected from the group consisting of: (i) CDR1 represented by amino acid residues (GYTFTSYW); (ii) CDR2 represented by amino acid residues (IYPSDSYT); and (iii) CDR3 represented by amino acid residues (TRSWRGNSFDY). In some embodiments, a light chain-coding region of an ssRNA consists of or comprises a nucleotide sequence that encodes at least one CDR (including, e.g., 1 CDR, 2 CDRs, and 3 CDRs) selected from the group consisting of (i) CDR1 represented by amino acid residues (QSLLNSGNQKNY); (ii) CDR2 represented by amino acid residues (WAS); and (iii) CDR3 represented by amino acid residues (QNDYSYPFT).

In some embodiments, a heavy-chain coding region of an ssRNA consists of or comprises a nucleotide sequence that encodes an amino acid sequence represented by amino acid residues 20-467 of SEQ ID NO: 1. In some embodiments, one or more amino acid modifications (e.g., to reduce immunogenicity and/or stability) may be present to one or more non-CDR regions of SEQ ID NO: 1. For example, in some embodiments, SEQ ID NO: 1 may comprise at least one or more (including, e.g., at least 1, at least 2, at least 3, at least 4, at least 5, at least 6, at least 7, at least 8, at least 9, at least 10, at least 15, at least 20, or more) amino acid modifications (including, e.g., amino acid insertions, deletions, and/or substitutions) to one or more non-CDR regions. In some embodiments, no more than 50 (including, e.g., no more than 40, no more than 30, no more than 20, no more than 10, or no more 5, or less) amino acid modifications may be present in one or more non-CDR regions of SEQ ID NO: 1. In some embodiments, a light-chain coding region of an ssRNA consists of or comprises a nucleotide sequence that encodes an amino acid sequence represented by amino acid residues 21-240 of SEQ ID NO: 2. In some embodiments, one or more amino acid modifications (e.g., to reduce immunogenicity and/or stability) may be present to one or more non-CDR regions of SEQ ID NO: 2. For example, in some embodiments, SEQ ID NO: 2 may comprise at least one or more (including, e.g., at least 1, at least 2, at least 3, at least 4, at least 5, at least 6, at least 7, at least 8, at least 9, at least 10, at least 15, at least 20, or more) amino acid modifications (including, e.g., amino acid insertions, deletions, and/or substitutions) to one or more non-CDR regions. In some embodiments, no more than 50 (including, e.g., no more than 40, no more than 30, no more than 20, no more than 10, or no more 5, or less) amino acid modifications may be present in one or more non-CDR regions of SEQ ID NO: 2.

In some embodiments, a heavy-chain coding region of an ssRNA consists of or comprises a nucleotide sequence that encodes the amino acid sequence of SEQ ID NO: 1. In some embodiments, a light-chain coding region of an ssRNA consists of or comprises a nucleotide sequence that encodes the amino acid sequence of SEQ ID NO: 2.

In some embodiments, a heavy-chain coding region of an ssRNA consists of or comprises a nucleotide sequence that encodes an amino acid sequence represented by amino acid residues 27-474 of SEQ ID NO: 3. In some embodiments, one or more amino acid modifications (e.g., to reduce immunogenicity and/or stability) may be present to one or more non-CDR regions of SEQ ID NO: 3. For example, in some embodiments, SEQ ID NO: 3 may comprise at least one or more (including, e.g., at least 1, at least 2, at least 3, at least 4, at least 5, at least 6, at least 7, at least 8, at least 9, at least 10, at least 15, at least 20, or more) amino acid modifications (including, e.g., amino acid insertions, deletions, and/or substitutions) to one or more non-CDR regions. In some embodiments, no more than 50 (including, e.g., no more than 40, no more than 30, no more than 20, no more than 10, or no more 5, or less) amino acid modifications may be present in one or more non-CDR regions of SEQ ID NO: 3. In some embodiments, a light-chain coding region of an ssRNA consists of or comprises a nucleotide sequence that encodes an amino acid sequence represented by amino acid residues 27-246 of SEQ ID NO: 4. In some embodiments, one or more amino acid modifications (e.g., to reduce immunogenicity and/or stability) may be present to one or more non-CDR regions of SEQ ID NO: 4. For example, in some embodiments, SEQ ID NO: 4 may comprise at least one or more (including, e.g., at least 1, at least 2, at least 3, at least 4, at least 5, at least 6, at least 7, at least 8, at least 9, at least 10, at least 15, at least 20, or more) amino acid modifications (including, e.g., amino acid insertions, deletions, and/or substitutions) to one or more non-CDR regions. In some embodiments, no more than 50 (including, e.g., no more than 40, no more than 30, no more than 20, no more than 10, or no more 5, or less) amino acid modifications may be present in one or more non-CDR regions of SEQ ID NO: 4.

In some embodiments, a heavy-chain coding region of an ssRNA consists of or comprises a nucleotide sequence that encodes the amino acid sequence of SEQ ID NO: 3. In some embodiments, a light-chain coding region of an ssRNA consists of or comprises a nucleotide sequence that encodes the amino acid sequence of SEQ ID NO: 4.

In some embodiments, a heavy chain-coding region of an ssRNA consists of or comprises a nucleotide sequence that encodes a full-length heavy chain of Zolbetuximab or Claudiximab (e.g., as described and/or exemplified herein). In some embodiments, a light chain-coding region of an ssRNA consists of or comprises a nucleotide sequence that encodes a full-length light chain of Zolbetuximab or Claudiximab.

In some embodiments, one or more ssRNAs can be used to encode a bispecific or multispecific antibody agent, which binds to two or more target molecules, e.g., one of which is a CLDN-18.2 polypeptide. For example, FIG. 12A illustrates exemplary bispecific antibody encoded by one or more ssRNAs. See also, e.g., Stadler et al. (2016) Oncoimmunology 5(3): e1091555; and/or in Stadler et al. (2017) Nature Medicine 23(7): 815-817. In some embodiments, a bivalent antibody agent may be encoded by a single ssRNA comprising a first coding region that encodes a single-chain variable fragment (scFv) that preferentially binds to a CLDN-18.2 polypeptide (relative to a CLDN18.1 polypeptide) and a second coding region that encodes a scFv for a second target (e.g., in some embodiments which may be a T cell receptor). In some embodiments, a bivalent antibody agent may be encoded by two separate ssRNAs: a first ssRNA comprising a coding region that encodes a scFv that preferentially binds to a CLDN-18.2 polypeptide (relative to a CLDN18.1 polypeptide) and a coding region that encodes a heavy chain antigen binding fragment (Fab) for a second target (e.g., in some embodiments which may be a T cell receptor); and a second ssRNA comprising a coding region that encodes a scFv targeting the CLDN-18.2 polypeptide and a coding region that encodes a light chain Fab for the same second target. In some embodiments, a bivalent antibody agent may be encoded by two separate ssRNAs: a first ssRNA comprising a coding region that encodes a scFv for a first target (e.g., in some embodiments which may be a T cell receptor) and a coding region that encodes a heavy chain antigen binding fragment (Fab) that preferentially binds to a CLDN-18.2 polypeptide (relative to a CLDN18.1 polypeptide); and a second ssRNA comprising a coding region that encodes a scFv for the same first target and a coding region that encodes a light chain Fab targeting the CLDN-18.2 polypeptide. Such first and second ssRNAs are then translated into subunits of an antibody and form a bispecific antibody in target cells.

Secretion signal-encoding region: In some embodiments, ssRNA(s) that encode a CLDN-18.2-targeting antibody agent) may comprise a secretion signal-encoding region. In some embodiments, such a secretion signal-encoding region allows a CLDN-18.2-targeting antibody agent encoded by one or more ssRNAs to be secreted upon translation by cells, e.g., present in a subject to be treated, thus yielding a plasma concentration of a biologically active a CLDN-18.2-targeting antibody agent. In some embodiments, a secretion signal-encoding region included in an ssRNA consists of or comprises a nucleotide sequence that encodes a non-human secretion signal. For example, in some embodiments, such a non-human secretion signal may be a murine secretion signal, which may in some embodiments be or comprises the amino acid sequence of MGWSCIILFLVATATGVHS or MESQTQVLMSLLFWVSGTCG. In some embodiments, a secretion signal-encoding region included in an ssRNA consists of or comprises a nucleotide sequence that encodes a human secretion signal, which may in some embodiments be or comprises the amino acid sequence of MRVMAPRTLILLLSGALALTETWAGS. In some embodiments, a secretion signal-encoding region included in an ssRNA encoding a heavy chain domain of a CLDN-18.2-targeting antibody agent may comprise a nucleotide sequence (i) that encodes a murine secretion signal amino acid sequence, which in some embodiments may be or comprise the amino acid sequence of MGWSCIILFLVATATGVHS; or that (ii) encodes a human secretion signal amino acid sequence, which in some embodiments may be or comprise the amino acid sequence of MRVMAPRTLILLLSGALALTETWAGS. In some embodiments, a secretion signal-encoding region included in an ssRNA encoding a light chain domain of a CLDN-18.2-targeting antibody agent may comprise a nucleotide sequence (i) that encodes a murine secretion signal amino acid sequence, which in some embodiments may be or comprise the amino acid sequence of MESQTQVLMSLLFWVSGTCG; or that (ii) encodes a human secretion signal amino acid sequence, which in some embodiments may be or comprise the amino acid sequence of MRVMAPRTLILLLSGALALTETWAGS.

In some embodiments, ssRNA(s) that encode a CLDN-18.2-targeting antibody agent may comprise at least one non-coding sequence element (e.g., to enhance RNA stability and/or translation efficiency). Examples of non-coding sequence elements include but are not limited to a 3′ untranslated region (UTR), a 5′ UTR, a cap structure for co-transcriptional capping of mRNA, a poly adenine (polyA) tail, and any combinations thereof.

UTRs (5′ UTRs and/or 3′UTRs): In some embodiments, a provided ssRNA can comprise a nucleotide sequence that encodes a 5′UTR of interest and/or a 3′ UTR of interest. One of skill in the art will appreciate that untranslated regions (e.g., 3′ UTR and/or 5′ UTR) of a mRNA sequence can contribute to mRNA stability, mRNA localization, and/or translational efficiency.

In some embodiments, a provided ssRNA can comprise a 5′ UTR nucleotide sequence and/or a 3′ UTR nucleotide sequence. In some embodiments, such a 5′ UTR sequence can be operably linked to a 3′ of a coding sequence (e.g., encompassing one or more coding regions). Additionally or alternatively, in some embodiments, a 3′ UTR sequence can be operably linked to 5′ of a coding sequence (e.g., encompassing one or more coding regions).

In some embodiments of any aspects described herein, 5′ and 3′ UTR sequences included in an ssRNA can consist of or comprise naturally occurring or endogenous 5′ and 3′ UTR sequences for an open reading frame of a gene of interest. Alternatively, in some embodiments, 5′ and/or 3′ UTR sequences included in an ssRNA are not endogenous to a coding sequence (e.g., encompassing one or more coding regions); in some such embodiments, such 5′ and/or 3′ UTR sequences can be useful for modifying the stability and/or translation efficiency of an RNA sequence transcribed. For example, a skilled artisan will appreciate that AU-rich elements in 3′ UTR sequences can decrease the stability of mRNA. Therefore, as will be understood by a skilled artisan, 3′ and/or 5′ UTRs can be selected or designed to increase the stability of the transcribed RNA based on properties of UTRs that are well known in the art.

For example, one skilled in the art will appreciate that, in some embodiments, a nucleotide sequence consisting of or comprising a Kozak sequence of an open reading frame sequence of a gene or nucleotide sequence of interest can be selected and used as a nucleotide sequence encoding a 5′ UTR. As will be understood by a skilled artisan, Kozak sequences are known to increase the efficiency of translation of some RNA transcripts, but are not necessarily required for all RNAs to enable efficient translation. In some embodiments, a provided ssRNA polynucleotide can comprise a nucleotide sequence that encodes a 5′ UTR derived from an RNA virus whose RNA genome is stable in cells. In some embodiments, various modified ribonucleotides (e.g., as described herein) can be used in the 3′ and/or 5′ UTRs, for example, to impede exonuclease degradation of the transcribed RNA sequence.

In some embodiments, a 5′ UTR included in an ssRNA may be derived from human α-globin mRNA combined with Kozak region.

In some embodiments, an ssRNA may comprise one or more 3′UTRs. For example, in some embodiments, an ssRNA may comprise two copies of 3′-UTRs derived from a globin mRNA, such as, e.g., alpha2-globin, alpha1-globin, beta-globin (e.g., a human beta-globin) mRNA. In some embodiments, two copies of 3′UTR derived from a human beta-globin mRNA may be used, e.g., in some embodiments which may be placed between a coding sequence of an ssRNA and a poly(A)-tail, to improve protein expression levels and/or prolonged persistence of an mRNA. In some embodiments, a 3′ UTR included in an ssRNA may be or comprise one or more (e.g., 1, 2, 3, or more) of the 3′UTR sequences disclosed in WO 2017/060314, the entire content of which is incorporated herein by reference for the purposes described herein. In some embodiments, a 3′-UTR may be a combination of at least two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see WO 2017/060314, herein incorporated by reference).

PolyA tail: In some embodiments, a provided ssRNA can comprise a nucleotide sequence that encodes a polyA tail. A polyA tail is a nucleotide sequence comprising a series of adenosine nucleotides, which can vary in length (e.g., at least 5 adenine nucleotides) and can be up to several hundred adenosine nucleotides. In some embodiments, a polyA tail is a nucleotide sequence comprising at least 30 adenosine nucleotides or more, including, e.g., at least 35, at least 40, at least 45, at least 50, at least 55, at least 60, at least 65, at least 70, at least 75, at least 80, at least 85, at least 90, at least 95, at least 100, or more adenosine nucleotides. In some embodiments, a polyA tail is or comprises a polyA homopolymeric tail. In some embodiments, a polyA tail may comprise one or more modified adenosine nucleosides, including, but not limited to, cordiocipin and 8-azaadenosine. In some embodiments, a polyA tail may comprise one or more non-adensoine nucleotides. In some embodiments, a polyA tail may be or comprise a disrupted or modified polyA tail as described in WO 2016/005324, the entire content of which is incorporated herein by reference for the purpose described herein. For example, in some embodiments, a polyA tail included in an ssRNA described herein may be or comprise a modified polyA sequence comprising: a linker sequence; a first sequence of at least 20 A consecutive nucleotides, which is 5′ of the linker sequence; and a second sequence of at least 20 A consecutive nucleotides, which is 3′ of the linker sequence. In some embodiments, a modified polyA sequence may comprise: a linker sequence comprising at least ten non-A nucleotides (e.g., T, G, and/or C nucleotides); a first sequence of at least 30 A consecutive nucleotides, which is 5′ of the linker sequence; and a second sequence of at least 70 A consecutive nucleotides, which is 3′ of the linker sequence.

5′ cap: In some embodiments, an ssRNA described herein may comprise a 5′ cap, which may be incorporated into such an ssRNA during transcription, or joined to such an ssRNA post-transcription. In some embodiments, an ssRNA may comprise a 5′ cap structure for co-transcriptional capping of mRNA. Examples of a cap structure for co-transcriptional capping are known in the art, including, e.g., as described in WO 2017/053297, the entire content of which is incorporated herein by reference for the purposes described herein. In some embodiments, a 5′ cap included in an ssRNA described herein is or comprises m7G(5′)ppp(5′)(2′OMeA)pG. In some embodiments, a 5′ cap included in an ssRNA described herein is or comprises a cap1 structure [m₂^{7,3′-O}Gppp(m₁^{2′-O})ApG].

In some embodiments, ssRNA(s) that encode a CLDN-18.2-targeting antibody agent may comprise at least one modified ribonucleotide, for example, in some embodiments to increase the stability of such ssRNA(s) and/or to decrease cytotoxicity of such ssRNAs. For example, in some embodiments, at least one of A, U, C, and G ribonucleotide of ssRNA(s) may be replaced by a modified ribonucleotide. For example, in some embodiments, some or all of cytidine residues present in an ssRNA may be replaced by a modified cytidine, which in some embodiments may be, e.g., 5-methylcytidine. Alternatively or additionally, in some embodiments, some or all of uridine residues present in an ssRNA may be replaced by a modified uridine, which in some embodiments may be, e.g., pseudoridine, such as, e.g., 1-methylpseudouridine. In some embodiments, all uridine residues present in an ssRNA is replaced by pseudouridine, e.g., 1-methylpseudouridine.

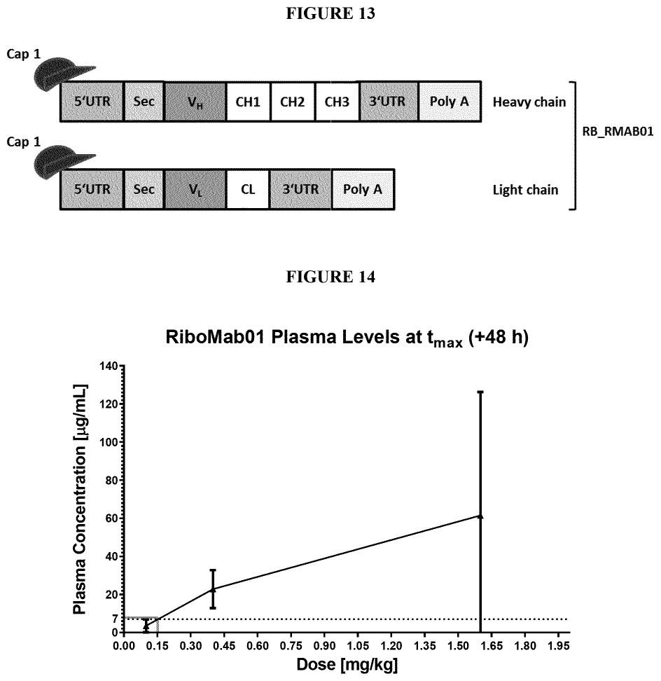

In some embodiments, an ssRNA encoding a heavy chain of a CLDN-18.2-targeting antibody agent comprises, in a 5′ to 3′ direction: (a) a 5′UTR-coding region; (b) a secretion signal-coding region; (c) a heavy chain-coding region; (d) a 3′ UTR-coding region; and (e) a polyA tail-coding region. See, for example, FIG. 13. In some embodiments, a 5′UTR-coding region is or comprises a sequence derived from human α-globin mRNA combined with Kozak region. In some embodiments, a secretion signal-coding region is or comprises a nucleotide sequence that encodes the amino acid sequence of MRVMAPRTLILLLSGALALTETWAGS. In some embodiments, a heavy chain-coding region encodes a V_{H} domain, a C_{H1} domain, a C_{H2} domain, and a C_{H3} domain of a CLDN-18.2-targeting antibody agent in an IgG form (e.g., ones as described herein, such as IMAB262, or an amino acid sequence represented by amino acid residues 27-474 of SEQ ID NO: 3. In some embodiments, a 3′ UTR-coding region is or comprises a combination of at least two sequence elements (FI element) derived from the “amino terminal enhancer of split” (AES) mRNA (called F) and the mitochondrial encoded 12S ribosomal RNA (called I). In some embodiments, a polyA tail-coding region is or comprises a modified polyA sequence (e.g., a polyA sequence of 100 adenosines disrupted by a linker sequence inserted immediately following 30 consecutive adenosines). In some embodiments, such an ssRNA comprises a 5′ cap structure comprising a CAP1 structure, or m₂^{7,3′-O}Gppp(m₁^{2′-O})ApG. In some embodiments, such an ssRNA comprises all uridines replaced by N1-methylpseudouridine.

In some embodiments, an ssRNA encoding a light chain of a CLDN-18.2-targeting antibody agent comprises, in a 5′ to 3′ direction: (a) a 5′UTR-coding region; (b) a secretion signal-coding region; (c) a light chain-coding region; (d) a 3′ UTR-coding region; and (e) a polyA tail-coding region. See, for example, FIG. 13. In some embodiments, a 5′UTR-coding region is or comprises a sequence derived from human α-globin mRNA combined with Kozak region. In some embodiments, a secretion signal-coding region is or comprises a nucleotide sequence that encodes the amino acid sequence of MRVMAPRTLILLLSGALALTETWAGS. In some embodiments, a light chain-coding region encodes a V_{L} domain and a C_{L} domain of a CLDN-18.2-targeting antibody agent in an IgG form (e.g., ones as described herein, such as IMAB262, or an amino acid sequence represented by amino acid residues 27-246 of SEQ ID NO: 4. In some embodiments, a 3′ UTR-coding region is or comprises a combination of at least two sequence elements (FI element) derived from the “amino terminal enhancer of split” (AES) mRNA (called F) and the mitochondrial encoded 12S ribosomal RNA (called I). In some embodiments, a polyA tail-coding region is or comprises a modified polyA sequence (e.g., a polyA sequence of 100 adenosines disrupted by a linker sequence inserted immediately following 30 consecutive adenosines). In some embodiments, such an ssRNA comprises a 5′ cap structure comprising a CAP1 structure, or m₂^{7,3′-O}Gppp(m₁^{2′-O})ApG. In some embodiments, such an ssRNA comprises all uridines replaced by N1-methylpseudouridine.

In some embodiments, ssRNA(s) is or comprises one or more single-stranded mRNAs.

In some embodiments, a composition comprises a single-stranded mRNA encoding a heavy chain (e.g., open reading frame, ORF) of an antibody agent targeting CLDN-18.2 (e.g., ones described herein) and a single-stranded mRNA encoding a light chain (e.g., open reading frame, ORF) of an antibody agent targeting CLDN-18.2 (e.g., ones described herein), which upon introduction into target cells, are translated into respective subunits and form a full IgG antibody in target cells. An exemplary drug substance is schematically presented in FIG. 13.

In some embodiments, an RNA drug substance is or comprises a combination of two ssRNAs, respectively, encoding a heavy (HC) and a light chain (LC) of an IgG CLDN-18.2 targeting antibody. In some embodiments, each of such two ssRNAs can be manufactured separately and an RNA drug substance can be prepared by mixing ssRNAs, respectively, encoding HC and LC of an IgG CLDN-18.2-targeting antibody in an appropriate weight ratio, e.g., a weight ratio such that the resulting molar ratio of HC- and LC-encoding single-stranded RNAs is about 1.5:1-1:1.5 for proper IgG formation.

In some embodiments, single-stranded RNAs encoding the HC and/or LC of a CLDN-18.2-targeting IgG antibody can comprise one or more non-coding sequence elements, for example, to enhance RNA stability and/or translational efficiency. For example, in some embodiments, such a single-stranded RNA oligonucleotide can comprise a cap structure, for example, a cap structure that can increase the resistance of RNA molecules to degradation by extracellular and intracellular RNases and leads to higher protein expression. In some embodiments, an exemplary cap structure is or comprises (m₂^{7,3′-O}Gppp(m₁^{2′-O}))ApG (cap1). In some embodiments, such a single-stranded RNA oligonucleotide can comprise one or more non-coding sequence elements at one or both of 5′ and 3′ untranslated regions (UTRs), for example, a naturally occurring sequence element at 5′ and 3′ UTRs that can significantly increase the intracellular half-life and the translational efficiency of the molecule (see, e.g., Holtkamp et al. 2006; Orlandini von Niessen et al. 2019). In some embodiments, an exemplary 5′ UTR sequence element is or comprises a characteristic sequence from human α-globin and a Kozak consensus sequence. In some embodiments, an exemplary 3′ UTR sequence element is or comprises a combination of two sequence elements (FI element) derived from the “amino terminal enhancer of split” (AES) mRNA (called F) and a mitochondrial encoded 12S ribosomal RNA (called I). See, e.g., WO 2017/060314, the entire content of which is incorporated herein by reference, for sequence information of exemplary 3′ UTR sequence element. In some embodiments, such a single-stranded RNA oligonucleotide can comprise a poly(A)-tail, for example, one that is designed to enhance RNA stability and/or translational efficiency. In some embodiments, an exemplary poly(A)-tail is or comprises a modified poly(A) sequence of 110 nucleotides in length including a stretch of 30 adenosine residues, followed by a 10 nucleotide linker sequence and another stretch of 70 adenosine residues (A30L70). In some embodiments, such a single-stranded RNA oligonucleotide can comprise one or more modified ribonucleotides. By way of example, only, in some embodiments, uridine of single-stranded RNAs can be replaced with a modified analog (e.g., N1-methylpseudouridine) to reduce and/or inhibit immune-modulatory activity and therefore enhances translation of the in vitro transcribed RNA.

In some embodiments, an RNA drug substance is or comprises a combination of a first single-stranded RNA having a construct of RNA-HC as disclosed in Table 2 below) and a second single-stranded RNA having a construct of RNA-LC as disclosed in Table 2 below. In some such embodiments, an RNA drug substance can be prepared by mixing the first and second single-stranded RNAs in a weight ratio of about 2:1.

TABLE 2
Exemplary constructs of single-stranded RNAs
encoding a CLDN-18.2-targeting IgG antibody

| | |
|---|---|
| Code DSI | RNA-HC (m₂^{7,3′-O}Gppp(m₁^{2′-O})ApG)-hAg-Kozak- |
| | aCLDN-18.2_HC-FI-A30L70 |
| Chemical class | Ribonucleic Acid (RNA) |
| Encoded protein | Heavy chain (ORF) of IgG CLDN-18.2-targeting |
| | antibody (e.g., IMAB362) |
| Laboratory code | RNA-LC (m₂^{7,3′-O}Gppp(m₁^{2′-O}))ApG)-hAg-Kozak- |
| DSI | aCLDN-18.2_LC -FI-A30L70 |
| Chemical class | Ribonucleic Acid (RNA) |
| Encoded protein | Light chain (ORF) of IgG CLDN-18.2-targeting |
| Laboratory code DS | antibody (e.g., IMAB362) RB_RMAB01 |
| Chemical class | Ribonucleic Acid (RNA) |
| Encoded protein | IgG CLDN-18.2-targeting RiboMab |

B. EXEMPLARY MANUFACTURING PROCESSES

Individual single-stranded RNAs can be produced by methods known in the art. For example, in some embodiments, single-stranded RNAs can be produced by in vitro transcription, for example, using a DNA template. A plasmid DNA used as a template for in vitro transcription to generate an ssRNA described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, ssRNAs (e.g., ones described herein) can be synthesized in the presence of modified ribonucleotide triphosphates. By way of example only, in some embodiments, N1-methylpseudouridine triphosphate (^{m1}ΨFTP) can be used to replace uridine triphosphate (UTP). As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where an ssRNA comprises a polyA tail, one of those skill in the art will appreciate that such a polyA tail may be encoded in a DNA template, e.g., by using an appropriately tailed PCR primer, or it can be added to an ssRNA after in vitro transcription, e.g., by enzymatic treatment (e.g., using a poly(A) polymerase such as an E. coli Poly(A) polymerase).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into an ssRNA during transcription (also known as co-transcriptional capping). In some embodiments, a 5′ cap analog for co-transcriptionally capping (e.g., ones described herein such as, e.g., m₂^{7,3′-O}Gppp(m₁^{2′-O})ApG) can be used during in vitro transcription. During polymerization, RNA is capped at the 5′-end with a 5′ cap analog (e.g., m₂^{7,3′-O}Gppp(m₁^{2′-O})ApG). In some embodiments, a GTP fed-batch procedure with multiple additions in the course of the reaction may be used to maintain a low concentration of GTP in order to effectively cap the RNA.

Following RNA transcription, a DNA template is digested. In some embodiments, digestion can be achieved with the use of DNase I under appropriate conditions.

In some embodiments, in-vitro transcribed single-stranded RNAs may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of single-stranded RNAs may further include one or more of the following steps: purification, mixing, filtration, and/or filling.

In some embodiments, ssRNAs can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, ssRNAs may be purified using magnetic bead-based purification, which in some embodiments may be or comprise magnetic bead-based chromatography. In some embodiments, ssRNAs may be purified using hydrophobic interaction chromatography (HIC) and/or diafiltration. In some embodiments, ssRNAs may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify ssRNAs according to methods described in WO 2017/182524, the entire content of which is incorporated herein by reference.

In some embodiments, a batch of ssRNAs may be further processed by one or more steps of filtration and/or concentration. For example, in some embodiments, ssRNA(s), for example, after removal of dsRNA contamination, may be further subject to diafiltration (e.g., in some embodiments by tangential flow filtration), for example, to adjust the concentration of ssRNAs to a desirable RNA concentration and/or to exchange buffer to a drug substance buffer.

In some embodiments where a CLDN-18.2-targeting antibody agent is encoded by a first ssRNA encoding a heavy chain of a CLDN-18.2-targeting antibody agent and a second ssRNA encoding a light chain of a CLDN-18.2-targeting antibody agent such that both, when both translated and expressed, form a full antibody, a batch of a first ssRNA and a batch of a second ssRNA, each after purification (e.g., as described herein) can be mixed in an appropriate ratio. For example, in some embodiments, such a first ssRNA batch and a second ssRNA batch may be mixed in a molar ratio of about 1:1.5 to about 1.5:1, e.g., in some embodiments in molar ratio of about 1:1.

In some embodiments, ssRNAs may be processed through 0.2 μm filtration before they are filled into appropriate containers.

In some embodiments, ssRNAs and compositions thereof may be manufactured in accordance with a process as described herein, or as otherwise known in the art.

In some embodiments, ssRNAs and compositions thereof may be manufactured at a large scale. For example, in some embodiments, a batch of ssRNAs can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of ssRNAs and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of an ssRNA manufacturing process, e.g., after in vitro transcription, and/or each purification step.

In some embodiments, the stability of ssRNAs (e.g., produced by in vitro transcription) and/or compositions comprising two or more RNAs (e.g., one encoding a HC of an antibody and another encoding a LC of the antibody) can be assessed under various test storage conditions, for example, at room temperatures vs. fridge or sub-zero temperatures over a period of time (e.g., at least 3 months, at least 6 months, at least 9 months, at least 12 months, or longer). In some embodiments, ssRNAs (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, ssRNAs (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, ssRNAs (e.g., ones described herein) and/or compositions thereof may be stored stable at room temperature (e.g., at about 25° C.) for at least 1 month or longer.

In some embodiments, one or more assessments as described in Example 11 may be utilized during manufacture, or other preparation or use of ssRNAs (e.g., as a release test).

In some embodiments, one or more quality control parameters may be assessed to determine whether ssRNAs described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of ssRNAs may be assessed for one or more features as described herein to determine next action step(s). For example, a batch of single stranded RNAs can be designated for one or more further steps of manufacturing and/or formulation and/or distribution if RNA quality assessment indicates that such a batch of single stranded RNAs meet or exceed the relevant acceptance criteria. Otherwise, an alternative action can be taken (e.g., discarding the batch) if such a batch of single stranded RNAs does not meet or exceed the acceptance criteria.

In some embodiments, a batch of ssRNAs that satisfy assessment results can be utilized for one or more further steps of manufacturing and/or formulation and/or distribution.

### IV. RNA Delivery Technologies

Provided ssRNAs (e.g., mRNA) may be delivered for therapeutic applications described herein using any appropriate methods known in the art, including, e.g., delivery as naked RNAs, or delivery mediated by viral and/or non-viral vectors, polymer-based vectors, lipid-based vectors, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery ssRNAs described herein.

In some embodiments, one or more ssRNAs can be formulated with lipid nanoparticles for delivery (e.g., in some embodiments by intravenous injection).

In some embodiments, lipid nanoparticles can be designed to protect ssRNAs (e.g., mRNA) from extracellular RNases and/or engineered for systemic delivery of the RNA to target cells (e.g., liver cells). In some embodiments, such lipid nanoparticles may be particularly useful to deliver ssRNAs (e.g., mRNA) when ssRNAs are intravenously administered to a subject in need thereof.

A. Lipid Nanoparticles

In some embodiments, provided ssRNAs (e.g., mRNA) may be formulated with lipid nanoparticles. In various embodiments, such lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles that may be useful in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles that may be useful in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, nucleic acids (e.g., ssRNAs), when present in provided lipid nanoparticles, are resistant in aqueous solution to degradation with a nuclease.

In some embodiments, lipid nanoparticles are liver-targeting lipid nanoparticles

In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

1. Helper Lipids

In some embodiments, a lipid nanoparticle for delivery of ssRNA(s) described herein comprises at least one helper lipid, which may be a neutral lipid, a positively charged lipid, or a negatively charged lipid. In some embodiments, a helper lipid is a lipid that are useful for increasing the effectiveness of delivery of lipid-based particles such as cationic lipid-based particles to a target cell. In some embodiments, a helper lipid may be or comprise a structural lipid with its concentration chosen to optimize LNP particle size, stability, and/or encapsulation.

In some embodiments, a lipid nanoparticle for delivery of ssRNA(s) described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid nanoparticles described herein. In some embodiments, a lipid nanoparticle for delivery of ssRNA(s) described herein comprises DSPC and/or cholesterol.

In some embodiments, a lipid nanoparticle for delivery of ssRNA(s) described herein comprises at least two helper lipids (e.g., ones described herein). In some such embodiments, a lipid nanoparticle may comprise DSPC and cholesterol.

2. Cationic Lipids

In some embodiments, a lipid nanoparticle for delivery of ssRNA(s) described herein comprises a cationic lipid. A cationic lipid is typically a lipid having a net positive charge. In some embodiments, a cationic lipid may comprise one or more amine group(s) which bear a positive charge. In some embodiments, a cationic lipid may comprise a cationic, meaning positively charged, headgroup. In some embodiments, a cationic lipid may have a hydrophobic domain (e.g., one or more domains of a neutral lipid or an anionic lipid) provided that the cationic lipid has a net positive charge. In some embodiments, a cationic lipid comprises a polar headgroup, which in some embodiments may comprise one or more amine derivatives such as primary, secondary, and/or tertiary amines, quaternary ammonium, various combinations of amines, amidinium salts, or guanidine and/or imidazole groups as well as pyridinium, piperizine and amino acid headgroups such as lysine, arginine, ornithine and/or tryptophan. In some embodiments, a polar headgroup of a cationic lipid comprises one or more amine derivatives. In some embodiments, a polar headgroup of a cationic lipid comprises a quaternary ammonium. In some embodiments, a headgroup of a cationic lipid may comprise multiple cationic charges. In some embodiments, a headgroup of a cationic lipid comprises one cationic charge. Examples of monocationic lipids include, but are not limited to 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradecoxy)propyl-(2-hydroxyethyl)-dimethylazanium bromide (DMRIE), didodecyl(dimethyl)azanium bromide (DDAB), 1,2-dioleyloxypropyl-3-dimethyl-hydroxyethyl ammonium bromide (DOME), 3P-[N-(N\N′-dimethylamino-ethane)carbamoyl]cholesterol (DC-Choi) and/or dioleyl ether phosphatidylcholine (DOEPC).

In some embodiments, a positively charged lipid structure described herein may also include one or more other components that may be typically used in the formation of vesicles (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the lipid into the lipid assembly. Examples of sterols include cholesterol, cholesteryl hemisuccinate, cholesteryl sulfate, or any other derivatives of cholesterol. Preferably, the at least one cationic lipid comprises DMEPC and/or DOTMA.

In some embodiments, a cationic lipid is ionizable such that it can exist in a positively charged form or neutral form depending on pH. Such ionization of a cationic lipid can affect the surface charge of the lipid particle under different pH conditions, which in some embodiments may influence plasma protein absorption, blood clearance, and/or tissue distribution as well as the ability to form endosomolytic non-bilayer structures. Accordingly, in some embodiments, a cationic lipid may be or comprise a pH responsive lipid. In some embodiments a pH responsive lipid is a fatty acid derivative or other amphiphilic compound which is capable of forming a lyotropic lipid phase, and which has a pKa value between pH 5 and pH 7.5. This means that the lipid is uncharged at a pH above the pKa value and positively charged below the pKa value. In some embodiments, a pH responsive lipid may be used in addition to or instead of a cationic lipid for example by binding one or more ssRNAs to a lipid or lipid mixture at low pH. pH responsive lipids include, but are not limited to, 1,2-dioieyioxy-3-dimethylamino-propane (DODMA).

In some embodiments, a lipid nanoparticle may comprise one or more cationic lipids as described in WO 2017/075531 (e.g., as presented in Tables 1 and 3 therein) and WO 2018/081480 (e.g., as presented in Tables 1-4 therein), the entire contents of each of which are incorporated herein by reference for the purposes described herein.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKₐ of about 6.0-6.5 (e.g., in one embodiment with an apparent pKₐ of approximately 6.25), resulting in an essentially fully positively charged molecule at an acidic pH (e.g., pH 5). In some embodiments, such an amino lipid, when incorporated in LNP, can confer distinct physicochemical properties that regulate particle formation, cellular uptake, fusogenicity and/or endosomal release of ssRNA(s). In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting LNP to a more neutral pH (e.g., pH 7.4) results in neutralization of the surface charge of the LNP. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders LNP comprising such an amino lipid fusogenic and allows the release of the RNA into the cytosol of the target cell.

In some embodiments, a cationic lipid that may be useful in accordance with the present disclosure has one of the structures set forth in Table 3 below:

TABLE 3
Exemplary cationic lipids

| No. | Structure |
|---|---|
| I-1 | 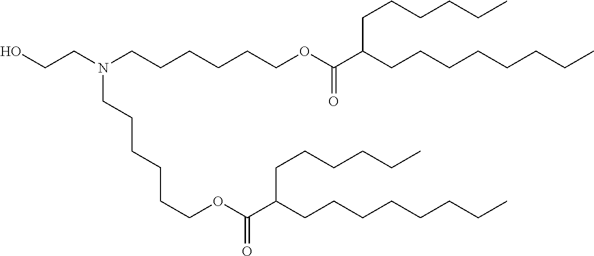 [SMILES: CCCCCCCCC(CCCCCC)C(=O)OCCCCCCN(CCO)CCCCCCOC(=O)C(CCCCCC)CCCCCCCC] |
| I-2 | 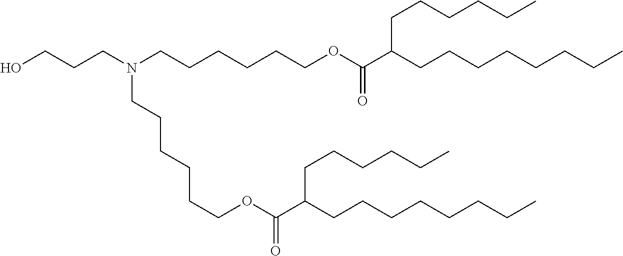 [SMILES: CCCCCCCCC(CCCCCC)C(=O)OCCCCCCN(CCCO)CCCCCCOC(=O)C(CCCCCC)CCCCCCCC] |
| I-3 | 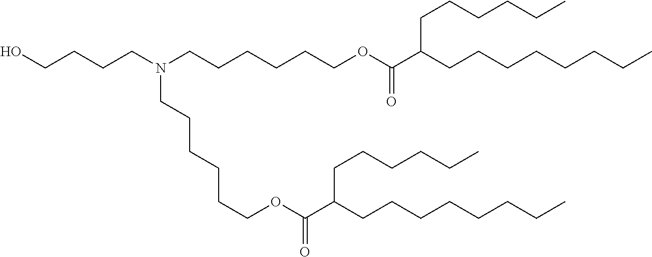 [SMILES: CCCCCCCCC(CCCCCC)C(=O)OCCCCCCN(CCCCO)CCCCCCOC(=O)C(CCCCCC)CCCCCCCC] |
| I-4 | 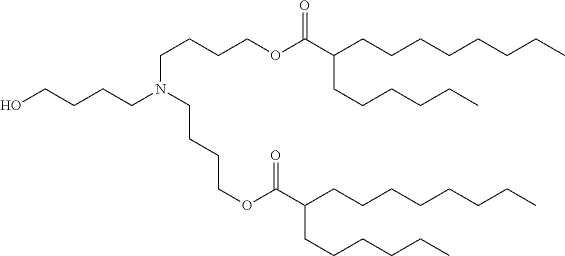 [SMILES: CCCCCCCCC(CCCCCC)C(=O)OCCCCN(CCCCO)CCCCOC(=O)C(CCCCCC)CCCCCCCC] |
| I-5 | 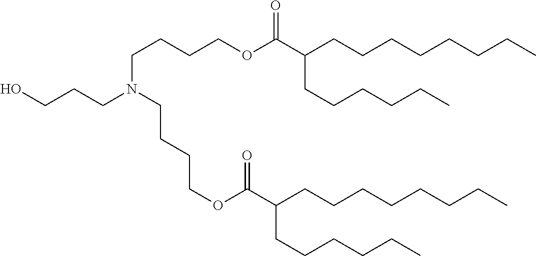 [SMILES: CCCCCCCCC(CCCCCC)C(=O)OCCCCN(CCCO)CCCCOC(=O)C(CCCCCC)CCCCCCCC] |
| I-6 | 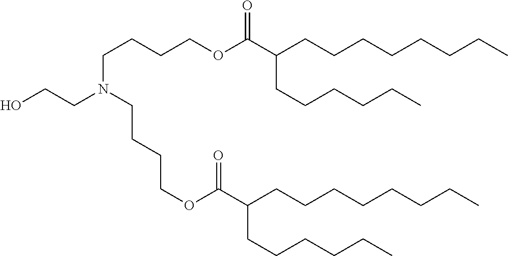 [SMILES: CCCCCCCCC(CCCCCC)C(=O)OCCCCN(CCO)CCCCOC(=O)C(CCCCCC)CCCCCCCC] |
| I-7 | 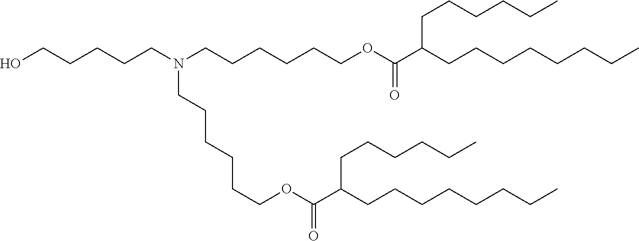 [SMILES: CCCCCCCCC(CCCCCC)C(=O)OCCCCCCN(CCCCCO)CCCCCCOC(=O)C(CCCCCC)CCCCCCCC] |
| I-8 | 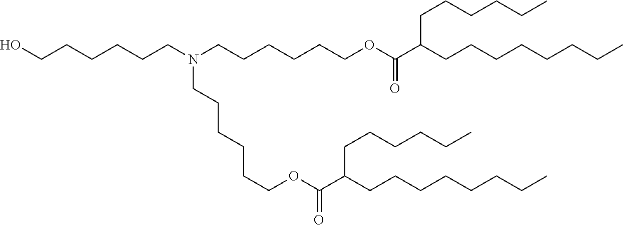 [SMILES: CCCCCCCCC(CCCCCC)C(=O)OCCCCCCN(CCCCCCO)CCCCCCOC(=O)C(CCCCCC)CCCCCCCC] |
| I-9 | 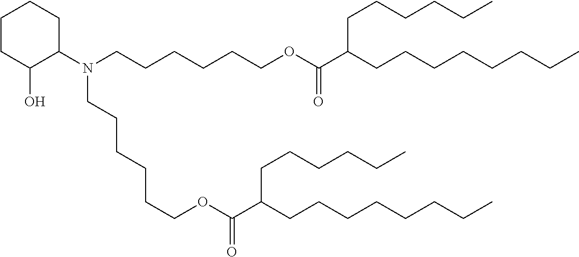 [SMILES: CCCCCCCCC(CCCCCC)C(=O)OCCCCCCN(CCCCCCOC(=O)C(CCCCCC)CCCCCCCC)C1CCCCC1O] |
| I-10 | 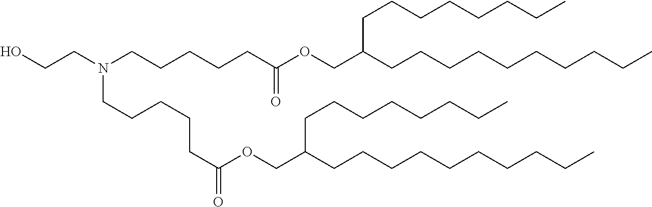 [SMILES: CCCCCCCCCCC(CCCCCCCC)COC(=O)CCCCCN(CCO)CCCCCC(=O)OCC(CCCCCCCC)CCCCCCCCCC] |
| I-11 | 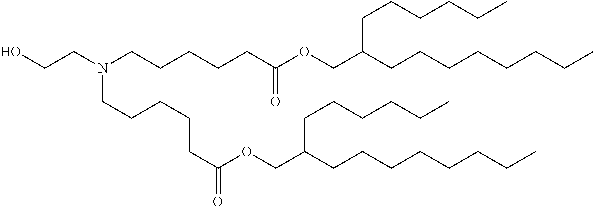 [SMILES: CCCCCCCCC(CCCCCC)COC(=O)CCCCCN(CCO)CCCCCC(=O)OCC(CCCCCC)CCCCCCCC] |
| I-12 | 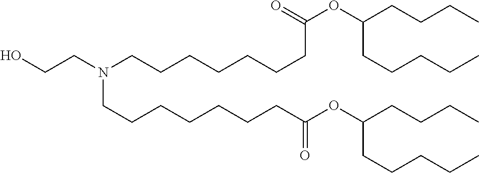 [SMILES: CCCCCC(CCCC)OC(=O)CCCCCCCN(CCO)CCCCCCCC(=O)OC(CCCC)CCCCC] |
| I-13 | 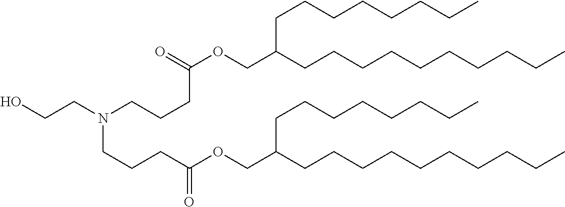 [SMILES: CCCCCCCCCCC(CCCCCCCC)COC(=O)CCCN(CCO)CCCC(=O)OCC(CCCCCCCC)CCCCCCCCCC] |
| I-14 | 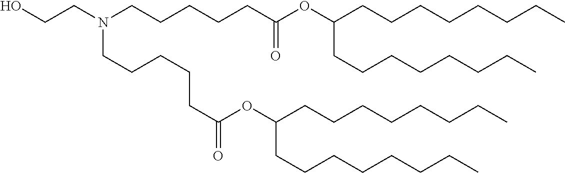 [SMILES: CCCCCCCCC(CCCCCCCC)OC(=O)CCCCCN(CCO)CCCCCC(=O)OC(CCCCCCCC)CCCCCCCC] |
| I-15 | 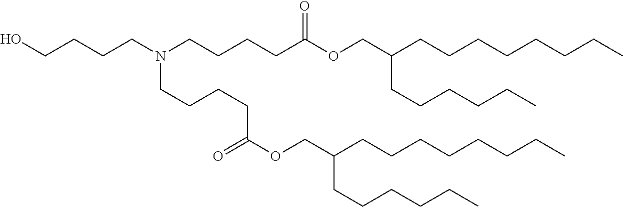 [SMILES: CCCCCCCCC(CCCCCC)COC(=O)CCCCN(CCCCO)CCCCC(=O)OCC(CCCCCC)CCCCCCCC] |
| I-16 | 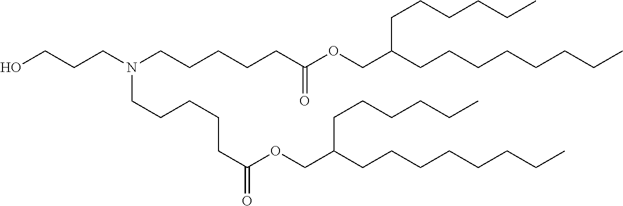 [SMILES: CCCCCCCCC(CCCCCC)COC(=O)CCCCCN(CCCO)CCCCCC(=O)OCC(CCCCCC)CCCCCCCC] |
| I-17 | 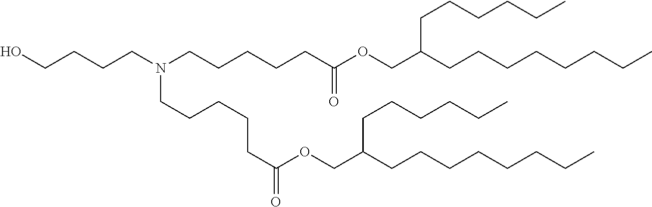 [SMILES: CCCCCCCCC(CCCCCC)COC(=O)CCCCCN(CCCCO)CCCCCC(=O)OCC(CCCCCC)CCCCCCCC] |
| I-18 | 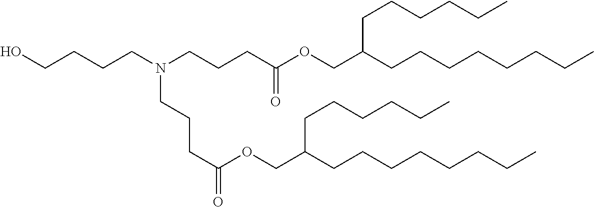 [SMILES: CCCCCCCCC(CCCCCC)COC(=O)CCCN(CCCCO)CCCC(=O)OCC(CCCCCC)CCCCCCCC] |
| I-19 | 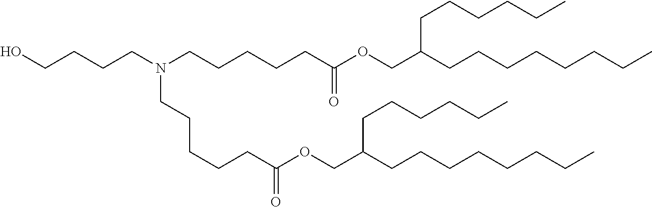 [SMILES: CCCCCCCCC(CCCCCC)COC(=O)CCCCCN(CCCCO)CCCCCC(=O)OCC(CCCCCC)CCCCCCCC] |
| I-20 | 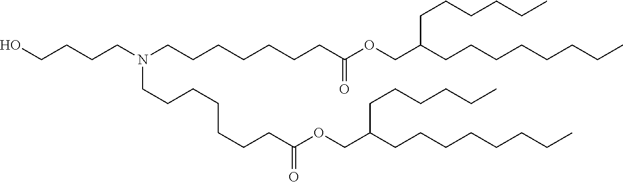 [SMILES: CCCCCCCCC(CCCCCC)COC(=O)CCCCCCCN(CCCCO)CCCCCCCC(=O)OCC(CCCCCC)CCCCCCCC] |
| I-21 | 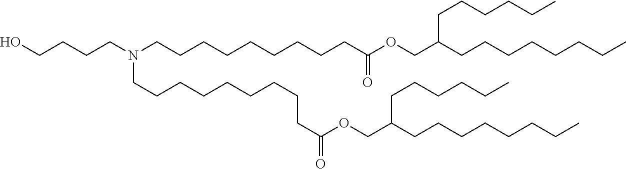 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 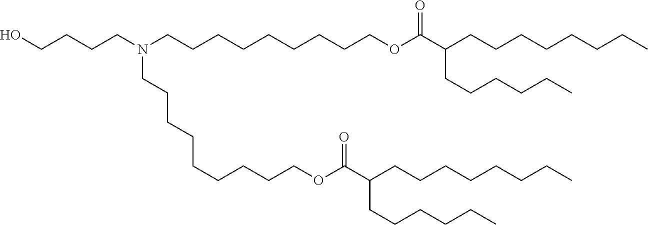 [SMILES: CCCCCCCCC(CCCCCC)C(=O)OCCCCCCCCCN(CCCCO)CCCCCCCCCOC(=O)C(CCCCCC)CCCCCCCC] |
| I-23 | 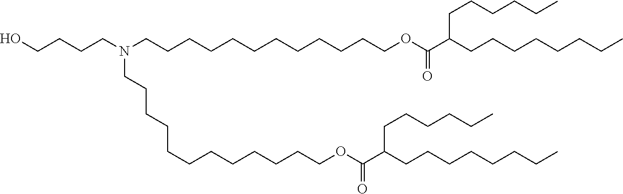 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 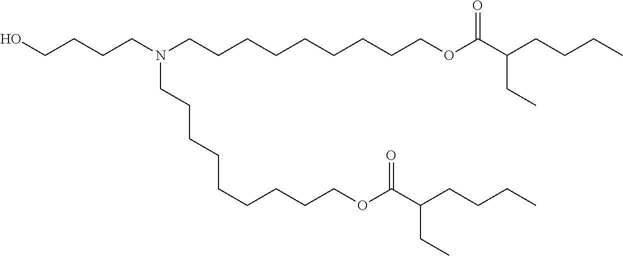 [SMILES: CCCCC(CC)C(=O)OCCCCCCCCCN(CCCCO)CCCCCCCCCOC(=O)C(CC)CCCC] |
| I-25 | 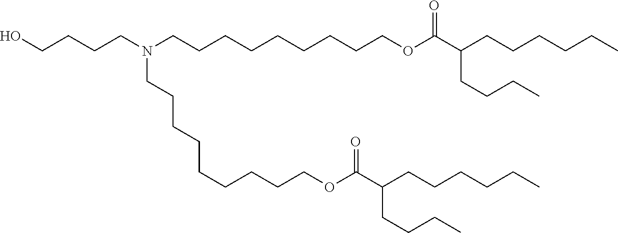 [SMILES: CCCCCCC(CCCC)C(=O)OCCCCCCCCCN(CCCCO)CCCCCCCCCOC(=O)C(CCCC)CCCCCC] |
| I-26 | 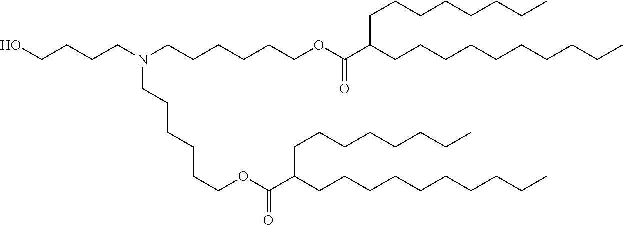 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 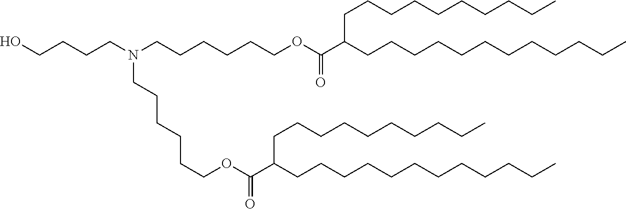 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 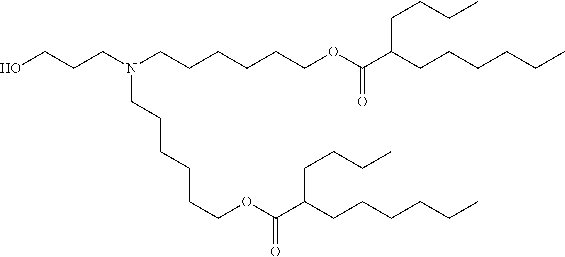 [SMILES: CCCCCCC(CCCC)C(=O)OCCCCCCN(CCCO)CCCCCCOC(=O)C(CCCC)CCCCCC] |
| I-29 | 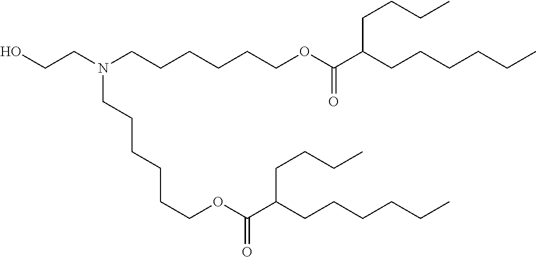 [SMILES: CCCCCCC(CCCC)C(=O)OCCCCCCN(CCO)CCCCCCOC(=O)C(CCCC)CCCCCC] |
| I-30 | 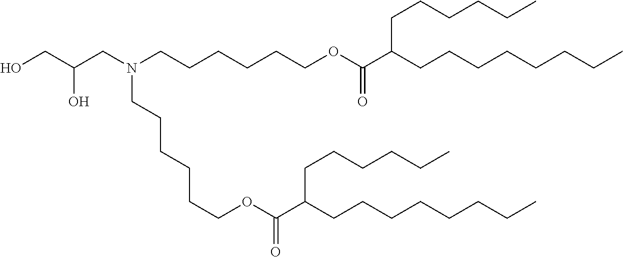 [SMILES: CCCCCCCCC(CCCCCC)C(=O)OCCCCCCN(CCCCCCOC(=O)C(CCCCCC)CCCCCCCC)CC(O)CO] |
| I-31 | 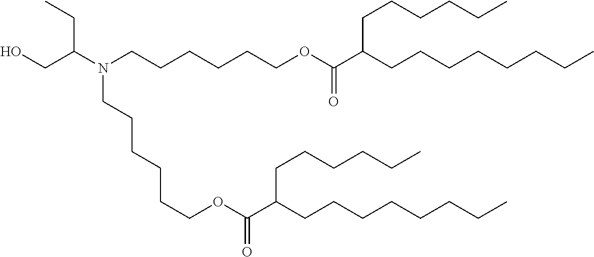 [SMILES: CCCCCCCCC(CCCCCC)C(=O)OCCCCCCN(CCCCCCOC(=O)C(CCCCCC)CCCCCCCC)C(CC)CO] |
| I-32 | 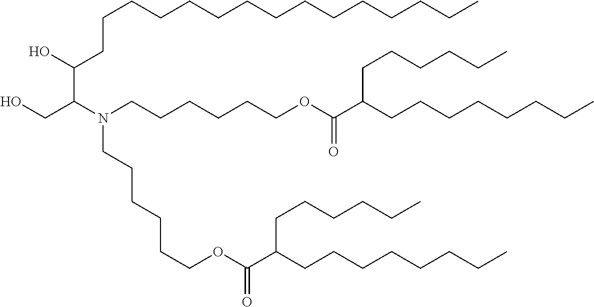 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 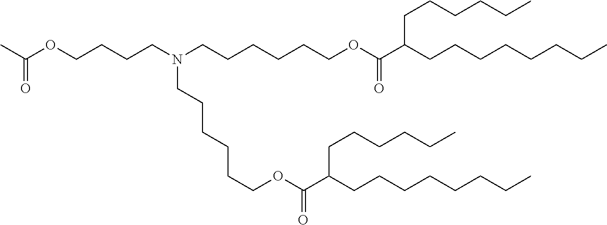 [SMILES: CCCCCCCCC(CCCCCC)C(=O)OCCCCCCN(CCCCCCOC(=O)C(CCCCCC)CCCCCCCC)CCCCOC(C)=O] |
| I-34 | 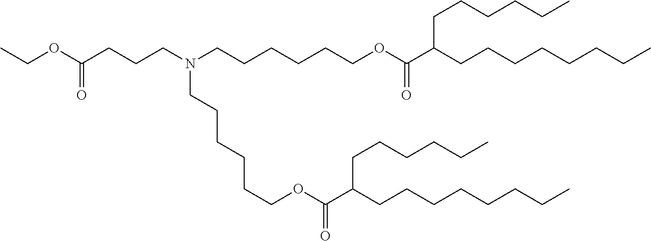 [SMILES: CCCCCCCCC(CCCCCC)C(=O)OCCCCCCN(CCCCCCOC(=O)C(CCCCCC)CCCCCCCC)CCCC(=O)OCC] |
| I-35 | 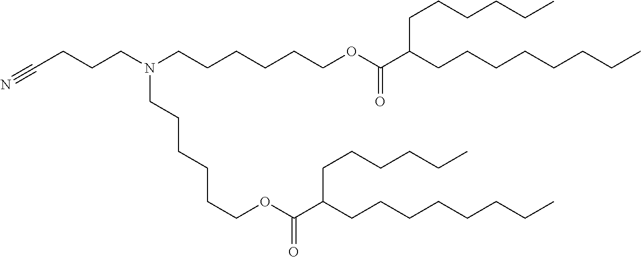 [SMILES: CCCCCCCCC(CCCCCC)C(=O)OCCCCCCN(CCCC#N)CCCCCCOC(=O)C(CCCCCC)CCCCCCCC] |
| I-36 | 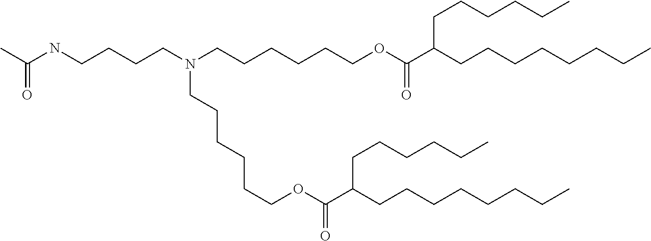 |
| I-37 | 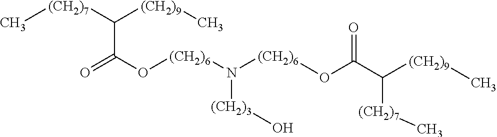 |
| I-38 | 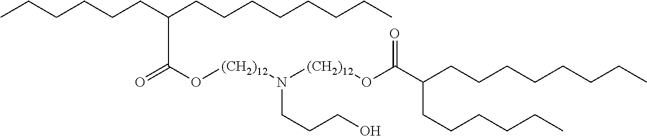 |
| I-39 | 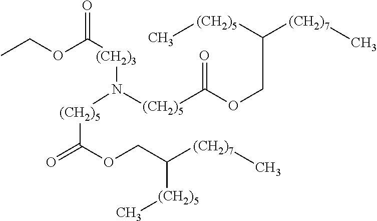 |
| I-40 | 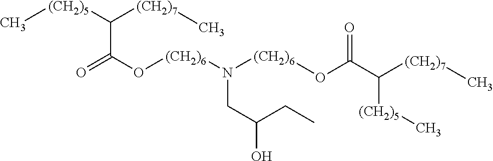 |
| I-41 | 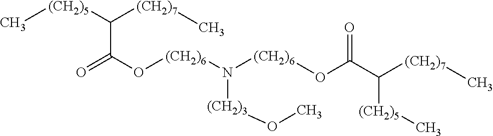 |
| I-42 | 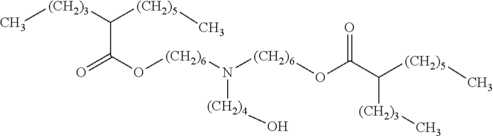 |
| I-43 | 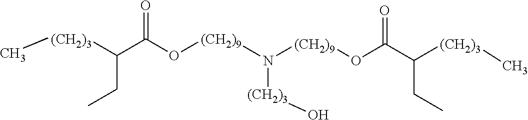 |
| I-44 | 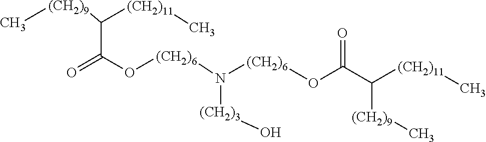 |
| I-45 | 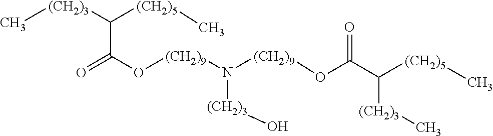 |
| I-46 | 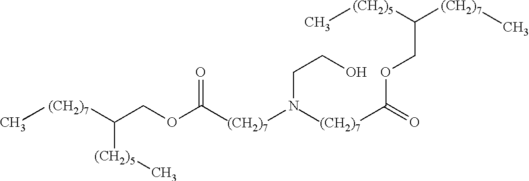 |
| I-47 | 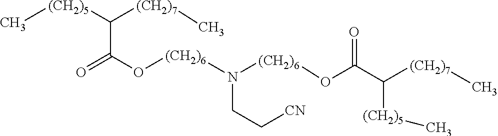 |
| I-48 | 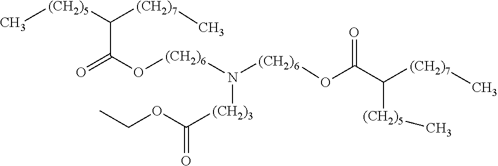 |
| I-49 | 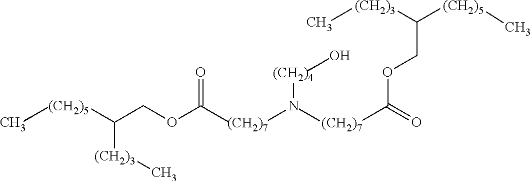 |

In certain embodiments, a cationic lipid that may be useful in accordance with the present disclosure is or comprises ((3-Hydroxypropyl)azanediyl)bis(nonane-9,1-diyl) bis(2-butyloctanoate) with a chemical structure shown in Example 14.

Cationic lipids may be used alone or in combination with neutral lipids, e.g., cholesterol and/or neutral phospholipids, or in combination with other known lipid assembly components.

3. Polymer-Conjugated Lipids

In some embodiments, a lipid nanoparticle for use in delivery of ssRNA(s) may comprise at least one polymer-conjugated lipid. A polymer-conjugated lipid is typically a molecule comprising a lipid portion and a polymer portion conjugated thereto.

In some embodiments, a polymer-conjugated lipid is a PEG-conjugated lipid. In some embodiments, a PEG-conjugated lipid is designed to sterically stabilize a lipid particle by forming a protective hydrophilic layer that shields the hydrophobic lipid layer. In some embodiments, a PEG-conjugated lipid can reduce its association with serum proteins and/or the resulting uptake by the reticuloendothelial system when such lipid particles are administered in vivo.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

Certain PEG-conjugated lipids (also known as PEGylated lipids) were clinically approved with safety demonstrated in clinical trials. PEG-conjugated lipids are known to affect cellular uptake, a prerequisite to endosomal localization and payload delivery. The present disclosure, among other things, provides an insight that the pharmacology of encapsulated nucleic acid can be controlled in a predictable manner by modulating the alkyl chain length of a PEG-lipid anchor. In some embodiments, the present disclosure, among other things, provides an insight that such PEG-conjugated lipids may be selected for an ssRNA/LNP drug product formulation to provide optimum delivery of ssRNAs to the liver. In some embodiments, such PEG-conjugated lipids may be designed and/or selected based on reasonable solubility characteristics and/or its molecular weight to effectively perform the function of a steric barrier. For example, in some embodiments, such a PEGylated lipid does not show appreciable surfactant or permeability enhancing or disturbing effects on biological membranes. In some embodiments, PEG in such a PEG-conjugated lipid can be linked to diacyl lipid anchors with a biodegradable amide bond, thereby facilitating fast degradation and/or excretion. In some embodiments, a LNP comprising a PEG-conjugated lipid retain a full complement of a PEGylated lipid. In the blood compartment, such a PEGylated lipid dissociates from the particle over time, revealing a more fusogenic particle that is more readily taken up by cells, ultimately leading to release of the RNA payload.

In some embodiments, a lipid nanoparticle may comprise one or more PEG-conjugated lipids or pegylated lipids as described in WO 2017/075531 and WO 2018/081480, the entire contents of each of which are incorporated herein by reference for the purposes described herein. For example, in some embodiments, a PEG-conjugated lipid that may be useful in accordance with the present disclosure can have a structure

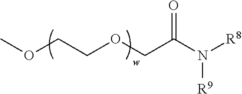

as described in WO 2017/075531, or a pharmaceutically acceptable salt, tautomer or stereoisomer thereof, wherein: R₈ and R₉ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R8 and R9 are each independently straight, saturated alkyl chains containing from 12 to 16 carbon atoms. In some embodiments, w has a mean value ranging from 43 to 53. In other embodiments, the average w is about 45. In some embodiments, a PEG-conjugated lipid is or comprises 2-[(Polyethylene glycol)-2000]-N,N-ditetradecylacetamide with a chemical structure as shown in Example 14.

In some embodiments, lipids that form lipid nanoparticles described herein comprise: a polymer-conjugated lipid; a cationic lipid; and a helper neutral lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, about 46-48 mol %, or about 47.2-47.8 mol % of the total lipids. In certain embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 40-46 mol %, about 40-44 mol %, or about 40-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in mol % of the total lipids; the non-steroid neutral lipid is present in 9-11 mol % of the total lipids; and the steroid neutral lipid is present in about 36-44 mol % of the total lipids. In many of such embodiments, a PEG-conjugated lipid is or comprises 2-[(polyethylene glycol)-2000]-N,N-ditetradecylacetamide or a derivative thereof. In many of such embodiments, a cationic lid is or comprises ((3-hydroxypropyl)azanediyl)bis(nonane-9,1-diyl) bis(2-butyloctanoate) or a derivative thereof. In many of such embodiments, a neutral lipid comprises DSPC and cholesterol, wherein DSPC is a non-steroid neutral lipid and cholesterol is a steroid neutral lipid.

B. Exemplary Methods of Making Lipid Nanoparticles

Lipids and lipid nanoparticles comprising nucleic acids and their method of preparation are known in the art, including, e.g., as described in U.S. Pat. No. 8,569,256, and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (LNP) are prepared at a total lipid to ssRNAs weight ratio of approximately 10:1 to 30:1. In some embodiments, such ssRNAs can be diluted to 0.2 mg/mL in acetate buffer.

In some embodiments, using an ethanol injection technique, a colloidal lipid dispersion comprising ssRNAs can be formed as follows: an ethanol solution comprising lipids, such as cationic lipids, neutral lipids, and polymer-conjugated lipids, is injected into an aqueous solution comprising ssRNAs (e.g., ones described herein).

In some embodiments, lipid and ssRNA solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous ssRNAs. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration, e.g., 0.2 μm filtration.

In some embodiments, particle size and/or internal structure of lipid nanoparticles (with or with ssRNs) may be monitored by appropriate techniques such as, e.g., small-angle X-ray scattering (SAXS) and/or transmission electron cryomicroscopy (CryoTEM).

### V. Provided Pharmaceutical Compositions Targeting Claudin-18.2

In some embodiments, a composition comprises provided ssRNA(s) that encodes a CLDN-18.2-targeting antibody agent. In some embodiments, such ssRNA(s) may be formulated with lipid nanoparticles (e.g., ones described herein) for administration to subject in needs thereof. Accordingly, one aspect provided herein relates to a pharmaceutical composition comprising provided ssRNA(s) that encodes a CLDN-18.2-targeting antibody agent and lipid nanoparticles (e.g., ones described herein), wherein such ssRNA(s) are encapsulated with the lipid nanoparticles.

In some embodiments where a pharmaceutical composition comprises a first ssRNA encoding a variable heavy chain (V_{H}) domain of a CLDN-18.2-targeting antibody agent (e.g., ones described herein) and a second ssRNA encoding a variable light chain (V_{L}) domain of the antibody agent (e.g., ones described herein), such a first ssRNA and a second ssRNA may be present in a molar ratio of about 1.5:1 to about 1:1.5, or in some embodiments in a molar ratio of about 1.2:1 to about 1:1.2, or in some embodiments in a molar ratio of about 1:1. In some embodiments, a first ssRNA encoding a variable heavy chain (V_{H}) domain of a CLDN-18.2-targeting antibody agent (e.g., ones described herein) and a second ssRNA encoding a variable light chain (V_{L}) domain of the antibody agent (e.g., ones described herein) may be present in a weight ratio of 3:1 to 1:1, or in some embodiments in a weight ratio of about 2:1.

In some embodiments, RNA content (e.g., one or more ssRNAs encoding a CLDN-18.2-targeting antibody agent) of a pharmaceutical composition described herein is present at a concentration of about 0.5 mg/mL to about 1.5 mg/mL, or about 0.8 mg/mL to about 1.2 mg/mL.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of ssRNAs encapsulated in LNPs, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., ssRNAs encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, a physician or veterinarian could start doses of active ingredients (e.g., ssRNAs encapsulated in lipid nanoparticles) employed in the pharmaceutical composition at levels lower than that required in order to achieve the desired therapeutic effect and gradually increase the dosage until the desired effect is achieved. For example, exemplary doses as described Example 8 may be used in preparing pharmaceutically acceptable dosage forms.

In some embodiments, a pharmaceutical composition described herein is formulated (e.g., for intravenous administration) to deliver an active dose that confers a plasma concentration of a CLDN-18.2-targeting antibody agent encoded by ssRNA(s) (e.g., ones described herein) that mediates pharmacological activity via its dominant mode of action, ADCC. For IMAB362, the dose-response correlation for ADCC is clinically well characterized and efficient lysis of CLDN-18.2+ cells through ADCC with an EC₉₅ of 0.3-28 μg/mL has been reported (Sahin et al. 2018). Thus, in some embodiments, a pharmaceutical composition described herein is formulated (e.g., for intravenous administration) to deliver an active dose that confers a plasma concentration of about 0.3-28 μg/mL of a CLDN-18.2-targeting antibody agent encoded by ssRNA(s) (e.g., ones described herein) that mediates pharmacological activity via its dominant mode of action, ADCC.

In some embodiments, a pharmaceutical composition described herein is formulated (e.g., for intravenous administration) to deliver one or more ssRNAs described herein (e.g., mRNA) encoding an antibody agent directed to CLDN-18.2 at a level expected to achieve level (e.g., plasma level and/or tissue level) of antibody above about 0.1 μg/mL; in some embodiments, above about 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2 μg/mL, 5 μg/mL, 8 μg/mL, 10 μg/mL, 15 μg/mL, 20 μg/mL, 25 μg/mL, or have a range up to and above what is observed with antibody administration.

In some embodiments, a pharmaceutical composition is formulated (e.g., for intravenous administration) to deliver a dose of 0.15 mg RNA/kg corresponding to approximately 7 μg/mL CLDN-18.2-targeting antibody agent at Cmax. FIG. 14 shows the dose-exposure correlation of RNA drug substance encoding CLDN-18.2-targeting antibody agent in cynomolgus monkey at tmax (48 hours). As will be appreciated by a skilled artisan, assuming that LNP-transfection efficacy and mRNA translation is comparable between cynomolgus monkey and humans (Coelho et al. 2013), a pharmaceutical composition, in some embodiments, is formulated (e.g., for intravenous administration) to deliver an appropriate dose corresponding to desirable plasma level of CLDN-18.2-targeting antibody agent encoded by ssRNA(s) as shown in FIG. 14.

In some embodiments, a pharmaceutical composition described herein is formulated (e.g., for intravenous administration) to deliver a dose of one or more ssRNAs (e.g., mRNA) encoding an antibody agent directed to CLDN-18.2 at a dose as described in Example 8, including, e.g., at a dose of 0.15 mg/kg, 0.2 mg/kg, 0.225 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.80 mg/kg, 0.85 mg/kg, 0.9 mg/kg, 0.95 mg/kg, 1.0 mg/kg, 1.25 mg/kg, 1.5 mg/kg, 1.75 mg/kg, 2.0 mg/kg, 2.25 mg/kg, 2.5 mg/kg, 2.75 mg/kg, 3.0 mg/kg, 3.25 mg/kg, 3.5 mg/kg, 4 mg/kg, 5 mg/kg, or higher. In some embodiments, a pharmaceutical composition described herein is formulated (e.g., for intravenous administration) to deliver a dose of one or more ssRNAs (e.g., mRNA) encoding an antibody agent directed to CLDN-18.2 at a dose of 1.5 mg/kg. In some embodiments, a pharmaceutical composition described herein is formulated to deliver a dose of one or more ssRNAs (e.g., mRNA) encoding an antibody agent directed to CLDN-18.2 at a dose of 5 mg/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition described herein may further comprises one or more active agents other than RNA (e.g., an ssRNA such as an mRNA) encoding a CLDN-18.2-targeting agent (e.g., antibody agent). For example, in some embodiments, such an other active agent may be or comprise a chemotherapeutic agent. In some embodiments, an exemplary chemotherapeutic agent may be or comprise a chemotherapeutic agent indicated for treatment of pancreatic cancer, including, e.g., but not limited to gemcitabine, and/or paclitaxel (e.g., nab-paclitaxel), folinic acid, fluorouracil, irinotecan, and/or oxaliplatin, etc. In some embodiments, an exemplary chemotherapeutic agent may be or comprise a chemotherapeutic agent indicated for treatment of biliary tract cancer, including, e.g., but not limited to gemcitabine and/or cisplatin.

In some embodiments, an active agent that may be included in a pharmaceutical composition described herein is or comprises a therapeutic agent administered in a combination therapy described herein. Pharmaceutical compositions described herein can be administered in combination therapy, i.e., combined with other agents. For example, in some embodiments, a combination therapy can include a provided pharmaceutical composition with at least one anti-inflammatory agent or at least one immunosuppressive agent. Examples of such therapeutic agents include but are not limited to one or more anti-inflammatory agents, such as a steroidal drug or a NSAID (nonsteroidal anti-inflammatory drug), aspirin and other salicylates, Cox-2 inhibitors, such as rofecoxib (Vioxx) and celecoxib (Celebrex), NSAIDs such as ibuprofen (Motrin, Advil), fenoprofen (Nalfon), naproxen (Naprosyn), sulindac (Clinoril), diclofenac (Voltaren), piroxicam (Feldene), ketoprofen (Orudis), diflunisal (Dolobid), nabumetone (Relafen), etodolac (Lodine), oxaprozin (Daypro), and indomethacin (Indocin). In some embodiments, such therapeutic agents may include agents leading to depletion or functional inactivation of regulatory T cells, e.g., low dose cyclophosphamid, anti-CTLA4 antibodies, anti-IL2 or anti-IL2-receptor antibodies.

In some embodiments, such therapeutic agents may include one or more chemotherapeutics, such as Taxol derivatives, taxotere, paclitaxel (e.g., nab-paclitaxel), gemcitabin, 5-Fluoruracil, doxorubicin (Adriamycin), cisplatin (Platinol), cyclophosphamide (Cytoxan, Procytox, Neosar), folinic acid, irinotecan, oxaliplatin. In some embodiments, pharmaceutical compositions described herein may be administered in combination with one or more chemotherapeutic agents, which can increase CLDN-18.2 expression level in a tumor of a cancer patient to be treated, e.g., by at least 10% or more, including, e.g., at least 20%, at least 30%, at least 40%, at least 50%, at least 60%, at least 70%, at least 80%, at least 90%, or more.

In some embodiments, pharmaceutical composition described herein may be administered in conjunction with radiotherapy and/or autologous peripheral stem cell or bone marrow transplantation.

In some embodiments, pharmaceutical compositions described herein may be administered in combination with one or more antibodies selected from anti-CD25 antibodies, anti-EPCAM antibodies, anti-EGFR, anti-Her2/neu, and anti-CD40 antibodies.

In some embodiments, pharmaceutical compositions described herein may be administered in combination with an anti-C3b(i) antibody in order to enhance complement activation.

In some embodiments, a pharmaceutical composition provided herein is a preservative-free, sterile RNA-LNP dispersion in an aqueous buffer for intravenous administration. In some embodiments, a RNA drug substance (e.g., ssRNAs described herein) included in a pharmaceutical composition is filled at 0.8 to 1.2 mg/mL, to a 5.0 mL nominal fill volume. A pharmaceutical composition is stored at −80 to −60° C.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

A. Identification and/or Characterization of Useful Components

To ensure appropriate quality of useful components (e.g., ssRNA(s) encoding CLDN-18.2-targeting antibody agent) in pharmaceutical compositions described herein, one or more quality assessments and/or relevant criteria (e.g., as described in Examples 11-12) may be performed and/or monitored.

Among other things, the present disclosure provides methods of characterizing one or more features of an ssRNA or composition thereof, which ssRNA encodes part or all of an antibody agent.

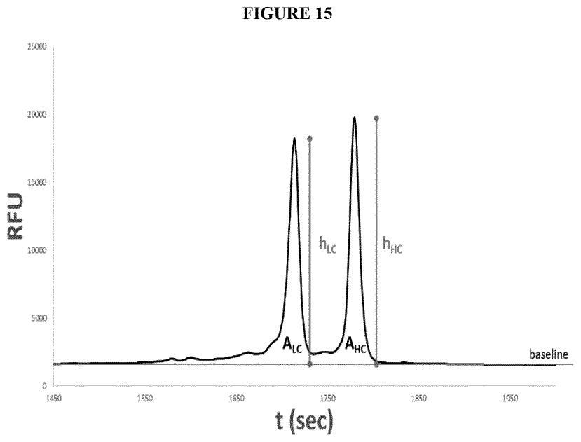

In some embodiments, RNA integrity assessment of ssRNA(s) (e.g., in some embodiments a composition comprising at least two ssRNAs each encoding a heavy chain or light chain of a CLDN-18.2-targeting antibody agent) can be performed by adaptation of a capillary gel electrophoresis assay. In some embodiments, the proportion of the area of the longer HC-coding RNA is evaluated to describe the integrity of both RNAs encoding different chains of a CLDN-18.2-targeting antibody agent. For example, an RNA composition comprising two or more RNAs can be analyzed by capillary gel electrophoresis, which gives an electropherogram as a result. By way of example only, an RNA composition comprising two different RNAs elutes in two separated peaks, for example, each corresponding to RNA encoding for a distinct chain (e.g., heavy chain or light chain) of an antibody. See, e.g., FIG. 15.

The present disclosure, among other things, provides an insight that the molecular ratio strongly influences this parameter and the specification of the mixture is set dependent on the molecular ratio measured by droplet digital PCR. This specification is reliant on a given mixture defined by the exact sequences and weight ratio.

Additionally or alternatively, in some embodiments, RNA ratio of an ssRNA encoding a heavy chain of a CLDN-18.2-targeting antibody agent to an ssRNA encoding a light chain of the CLDN-18.2-targeting antibody agent can be measured by droplet digital PCR.

Additionally or alternatively, in some embodiments, residual DNA template and residual dsRNA are measured as in-process controls with acceptance criteria on the level of the drug substance intermediates to ensure individual RNA quality before mixing to the drug substance, for example, before mixing two ssRNAs encoding different chains of a CLDN-18.2-targeting antibody agent. In some embodiments, relevant acceptance criteria are used for in-process controls of the quality of individual ssRNAs.

Additionally or alternatively, in some embodiments, residual host cell DNA and/or host cell protein may be measured in compositions comprising ssRNAs.

B. Characterization of Effective Delivery (e.g., Plasma Concentration)

In some embodiments, compositions and components thereof can be assessed to determine their efficacy. In some embodiments, primary pharmacodynamics and/or pharmacokinetics of pharmaceutical compositions described herein in vitro and/or in vivo can be determined. Examples of useful pharmacokinetics measurements may include one or more parameters:
Cₘₐₓ corresponds to maximum (or peak) plasma/serum concentration that a drug achieves in a specified compartment or test area of the body after the drug has been administered and before the administration of a second dose. The related pharmacokinetics parameter tₘₐₓ is the time at which the Cmax is observed.
Cₘᵢₙ corresponds to minimum plasma/serum concentration that a drug achieves after dosing.
C_{trough} corresponds to trough plasma concentration at the end of a dosing interval at steady state (typically taken directly before next administration)
Area under the curve (AUC) is the definite integral of a curve that describes variation of a drug concentration in blood plasma as a function of time. The AUC (from zero to infinity) represents the total drug exposure across time.

In some embodiments, functional assembly of a CLDN-18.2-targeting antibody agent encoded by ssRNAs can be determined in vitro and in vivo in a dose-dependent manner, e.g., as described in Example 6.

In some embodiments, binding specificity, mediation of ADCC and CDC, and/or anti-tumor activity of CLDN-18.2-targeting antibody agent encoded by ssRNA(s) described herein can be determined, e.g., as described in Examples 1-4.

Among other things, the present disclosure provides a method comprising a step of: determining one or more features of an antibody agent expressed from at least one mRNA introduced into cells, wherein such at least one mRNA comprises one or more of features of at least one or more ssRNA comprising a coding region that encodes an antibody agent that binds preferentially to a Claudin-18.2 (CLDN-18.2) polypeptide relative to a Claudin-18.1 polypeptide, wherein such one or more features comprises: (i) protein expression level of an antibody agent; (ii) binding specificity of an antibody agent to CLDN-18.2; (iii) efficacy of an antibody agent to mediate target cell death through ADCC; and (iv) efficacy of an antibody agent to mediate target cell death through complement dependent cytotoxicity (CDC).

In some embodiments, provided herein is a method of characterizing a pharmaceutical composition targeting CLDN-18.2. Such a method comprises steps of: (a) contacting cells with at least one composition or pharmaceutical composition described herein (which encodes part or all of a CLDN-18.2-targeting antibody agent); and detecting an antibody agent produced by the cells. In some embodiments, the cells may be or comprise liver cells.

In some embodiments, such a method may further comprise determining one or more features of an antibody agent expressed from one or more ssRNAs described herein, wherein such one or more features comprises: (i) protein expression level of the antibody agent; (ii) binding specificity of the antibody agent to a CLDN-18.2 polypeptide; (iii) efficacy of the antibody agent to mediate target cell death through ADCC; and (iv) efficacy of the antibody agent to mediate target cell death through complement dependent cytotoxicity (CDC). In some embodiments, a step of determining one or more features of an antibody agent expressed from one or more ssRNAs described herein may comprise comparing such features of the CLDN-18.2-targeting antibody agent with that of a reference CLDN-18.2-targeting antibody.

In some embodiments, a step of determining one or more features of an antibody agent expressed from one or more ssRNAs described herein may comprise assessing the protein expression level of the antibody agent above a threshold level. For example, in some embodiments, a threshold level corresponds to a therapeutically relevant plasma concentration.

In some embodiments, a step of determining one or more features of an antibody agent expressed from one or more ssRNAs described herein may comprise assessing binding of the antibody agent to a CLDN-18.2 polypeptide. In some embodiments, such binding assessment may comprise determining binding of the antibody agent to a CLDN-18.2 polypeptide relative to its binding to a CLDN18.1 polypeptide. In some embodiments, such binding assessment may comprise determining a binding preference profile of the antibody agent at least comparable to that of a reference CLDN-18.2-targeting antibody. For example, in some embodiments, a reference CLDN-18.2-targeting antibody is Zolbetuximab or Claudiximab.

In some embodiments, a provided method of characterizing a pharmaceutical composition targeting CLDN-18.2 or components thereof may further comprise characterizing an antibody agent expressed from one or more ssRNAs described herein as a CLDN-18.2-targeting antibody agent if the antibody agent comprises the following features: (a) protein level of the antibody agent expressed by the cells above a threshold level; (b) preferential binding of the antibody agent to CLDN-18.2 relative to CLDN18.1; and (c) killing of at least 50% target cells (e.g., cancer cells) mediated by ADCC and/or CDC.

In some embodiments, a provided method of characterizing a pharmaceutical composition targeting CLDN-18.2 or components thereof may further comprise characterizing an antibody agent expressed from one or more ssRNAs described herein as a Zolbetuximab or Claudiximab-equivalent antibody if tested features of the antibody are at least comparable to that of Zolbetuximab or Claudiximab.

In some embodiments involving a step of determining one or more features of an antibody agent expressed from one or more ssRNAs described herein, such a step may comprise determining one or more of the following features:
whether, when assessed 48 hours after contacting or administering, cells express a CLDN-18.2-targeting antibody agent encoded by at least one ssRNA;
whether the antibody agent expressed by the cells binds preferentially to a CLDN-18.2 polypeptide relative to a CLDN18.1 polypeptide;
whether the antibody agent expressed by the cells exhibit comparable target specificity to CLDN-18.2 as observed in a flow cytometric binding assay with a reference CLDN-18.2-targeting monoclonal antibody;
whether, when assessed 48 hours after incubating immune effector cells (e.g., PBMC cells) and CLDN-18.2 positive cells or CLDN-18.2 negative control cells in the presence of the antibody agent, the CLDN-18.2 positive cells, not the control cells, were lysed;
whether the antibody agent expressed by the cells exhibit at least comparable ADCC profile of targeted CLDN-18.2 positive cells as observed with a reference CLDN-18.2-targeting monoclonal antibody in the same concentration; and
whether, when assessed 2 hours after incubating CLDN-18.2 positive cells or CLDN-18.2 negative control cells with human serum in the presence of the antibody agent, the CLDN-18.2 positive cells, not the control cells, were lysed.

In some embodiments, cells used in provided methods of characterizing a pharmaceutical composition targeting CLDN-18.2 or components thereof are present in vivo, e.g., in a subject (e.g., a mammalian subject such as a mammalian non-human subject, e.g., a mouse or monkey subject). In some such embodiments, a step of determining one or more features of an antibody agent expressed from one or more ssRNAs described herein may include determining antibody level in one or more tissues in such a subject. In some embodiments, such a method of characterizing may further comprise administering a composition or pharmaceutical composition described herein to a group of animal subjects each bearing a human CLDN-18.2 positive xenograft tumor to determine anti-tumor activity, if such a composition or pharmaceutical composition is characterized as a CLDN-18.2-targeting antibody agent.

Also within the scope of the present disclosure includes a method of manufacture, which comprises steps of:
(A) determining one or more features of an ssRNA or composition thereof, which ssRNA encodes part or all of an antibody agent, which one or more features are selected from the group consisting of:
   (i) length and/or sequence of the ssRNA;
   (ii) integrity of the ssRNA;
   (iii) presence and/or location of one or more chemical moieties of the ssRNA;
   (iv) extent of expression of the antibody agent when the ssRNA is introduced into a cell;
   (v) stability of the ssRNA or composition thereof;
   (vi) level of antibody agent in a biological sample from an organism into which the ssRNA has been introduced;
   (vii) binding specificity of the antibody agent expressed from the ssRNA, optionally to CLDN-18.2 and optionally relative to CLDN18.1;
   (viii) efficacy of the antibody agent to mediate target cell death through ADCC;
   (ix) efficacy of the antibody agent to mediate target cell death through complement dependent cytotoxicity (CDC);
   (x) lipid identity and amount/concentration within the composition;
   (xi) size of lipid nanoparticles within the composition;
   (xii) polydispersity of lipid nanoparticles within the composition;
   (xiii) amount/concentration of the ssRNA within the composition;
   (xiv) extent of encapsulation of the ssRNA within lipid nanoparticles; and
   (xv) combinations thereof;
(B) comparing such one or more features of the ssRNA or composition thereof with that of an appropriate reference standard; and
(C) (i) designating the ssRNA or composition thereof for one or more further steps of manufacturing and/or distribution if the comparison demonstrates that the ssRNA or composition thereof meets or exceeds the reference standard; or
   (ii) taking an alternative action if the comparison demonstrates that the ssRNA or composition thereof does not meet or exceed the reference standard.

In some embodiments, a reference standard can be any quality control standard, including, e.g., a historical reference, a set specification. As will be understood by a skilled artisan, in some embodiments, a direct comparison is not required. In some embodiments, a reference standard is an acceptance criterion based on, for example, physical appearance, lipid identity and/or content, LNP size, LNP polydispersity, RNA encapsulation, RNA length, identity (as RNA), integrity, sequence, and/or concentration, pH, osmolality, RNA ratio (e.g., ratio of a HC RNA to a LC RNA), potency, bacterial endotoxins, bioburden, residual organic solvent, osmolality, pH, and combinations thereof.

In some embodiments, pharmaceutical compositions described herein can be determined by one or more potency assays, namely, e.g., but not limited to in vitro translation, enzyme-linked immunosorbent assay (ELISA) and/or a T-cell activation bioassay. For example, in some embodiments, expression of a CLDN-18.2-targeting antibody encoded by RNA compositions (e.g., ones described herein) in cells can be measured in the culture supernatant of lipofected production cells by ELISA. In some such embodiments, supernatant of lipofected production cells may be added to a co-culture of CLDN-18.2-expressing target cells and FcRIIIa-positive luciferase reporter cells as effector cells. Simultaneous binding of the antibody to CLDN-18.2 and to the FcγRIIIa receptor leads to the activation of the effector cells and results in luciferase expression, which is quantified by luminescence readout.

In some embodiments of a method of manufacture, when an ssRNA (e.g., ones described herein) is assessed and one or more features of the ssRNA meets or exceeds an appropriate reference standard, such an ssRNA is designated for formulation, e.g., in some embodiments involving formulation with lipid particles described herein.

In some embodiments of a method of manufacture, when a composition comprising an ssRNA (e.g., ones described herein) is assessed and one or more features of the composition meets or exceeds an appropriate reference standard, such a composition is designated for release and/or distribution of the composition.

In some embodiments of a method of manufacture, when an ssRNA (e.g., ones described herein) is designated for formulation, and/or a composition comprising an ssRNA (e.g., ones described herein) is designated for release and/or distribution of the composition, such a method may further comprise administering the formulation and/or composition to a group of animal subjects each bearing a human CLDN-18.2 positive xenograft tumor to determine anti-tumor activity.

Methods of producing a CLDN-18.2-targeting antibody agent are also within the scope of the present disclosure. In some embodiments, a method of producing a CLDN-18.2-targeting antibody agent comprises administering to cells a composition comprising at least one ssRNA (e.g., ones as described herein) comprising one or more coding regions that encode a CLDN-18.2-targeting antibody agent so that such cells express and secrete a CLDN-18.2-targeting antibody agent encoded by such ssRNA(s). In some embodiments, cells to be administered or targeted are or comprise liver cells.

In some embodiments, cells are present in a cell culture.

In some embodiments, cells are present in a subject. In some such embodiments, a pharmaceutical composition described herein may be administered to a subject in need thereof. In some embodiments, such a pharmaceutical composition may be administered to a subject such that a CLDN-18.2-targeting antibody agent is produced at a therapeutically relevant plasma concentration. In some embodiments, a therapeutically relevant plasma concentration is sufficient to mediate cancer cell death through antibody-dependent cellular cytotoxicity (ADCC). For example, in some embodiments, a therapeutically relevant plasma concentration is 0.3-28 μg/mL.

VI. Exemplary Cancers Associated with High Expression of CLDN-18.2

### A. Solid Tumors

Cancer is the second leading cause of death globally and is expected to be responsible for an estimated 9.6 million deaths in 2018 (Bray et al. 2018). In general, once a solid tumor has metastasized, with a few exceptions such as germ cell and some carcinoid tumors, 5-year survival rarely exceeds 25%.

Treatment of Advanced and Metastatic Solid Tumors

Refinements in conventional therapies such as chemotherapy, radiotherapy, surgery, and targeted therapies and recent advances in immunotherapies have improved outcomes in patients with advanced solid tumors. In the last few years, the Food and Drug Administration (FDA) and European Medicines Agency (EMA) have approved eight checkpoint inhibitors (one monoclonal antibody targeting the CTLA-4 pathway, ipilimumab, and seven antibodies targeting programmed death receptor/ligand [PD/PD-L1], including atezolizumab, avelumab, durvalumab, nivolumab, cemiplimab and pembrolizumab), for the treatment of patients with multiple cancer types, mainly solid tumors. These approvals have dramatically changed the landscape of cancer treatment. However, certain cancers such as pancreatic adenocarcinoma or metastatic biliary tract cancers still do not yet benefit from existing immunotherapies. This phenomenon is multifactorial, attributed to pancreatic ductal adenocarcinoma (PDAC)'s systemic and aggressive nature, its complex mutational landscape, its desmoplastic stroma, and a potently immunosuppressive tumor microenvironment.

The poor prognosis of these two cancer types highlights the need for additional treatment approaches. The present disclosure, among other things, provides an insight that CLDN-18.2 represents a particularly useful tumor-associated antigen against which therapies may be targeted. To date, no therapy targeting CLDN-18.2 has been approved for any cancer indication. Accordingly, in some embodiments, the present disclosures provides an insight that RNA-encoded antibodies targeting CLDN-18.2 can induce ADCC and/or CDC and/or augment cytotoxic effect(s) of chemotherapy and/or other anti-cancer therapy, thus translating into prolonged progression-free and/or overall survival, e.g., relative to the individual therapies administered alone and/or to another appropriate reference.

### B. Pancreatic Ductal Adenocarcinoma

Pancreatic ductal adenocarcinoma (PDAC) is the most prevalent neoplastic disease of the pancreas accounting for more than 90% of all pancreatic malignancies (Kleeff et al. 2016). To date, PDAC is the fourth most frequent cause of cancer-related deaths worldwide with a 5-year overall survival of less than 8% (Siegel et al. 2018). The incidence of PDAC is expected to rise further in the future, and projections indicate a more than 2-fold increase in the number of cases within the next 10 years, both in terms of new diagnoses as well as in terms of PDAC-related deaths in the United States and European countries (Quante et al. 2016; Rahib et al. 2014; Cancer Research UK).

The efficacy and outcome of PDAC treatment are largely determined by the stage of disease at the time of diagnosis. Surgical resection followed by adjuvant chemotherapy is the only possibly curative therapy available, yet only 10-20% of PDAC patients present with resectable PDAC stages, while the residual 80-90% show locally advanced, non-resectable stages or—in the majority of cases—distant metastases (Gillen et al. 2010; Werner et al. 2013). Systemic chemotherapy is commonly employed as a first-line treatment in patients with non-resectable or borderline-resectable tumors. This encompasses nucleoside analogues, including gemcitabine and capecitabine, or the pyrimidine analogue 5-fluorouracil either in monotherapy settings or in combination with other treatment modalities, such as radiotherapy (Werner et al. 2013; Manji et al. 2017; Teague et al. 2015). FOLFIRINOX, a poly-chemotherapeutic regimen composed of folinic acid, 5-fluorouracil, irinotecan, and oxaliplatin, has been reported to nearly double median survival in the metastasized stage as compared to gemcitabine alone (Conroy et al. 2011), and the combination of gemcitabine and a nanoparticle albumin-bound paclitaxel (nab-paclitaxel) has also been shown to significantly improve overall survival (Von Hoff et al. 2013). These treatments are associated with relatively high toxicity, thus often preventing their application in elderly patients and/or patients with poor performance status, however, overall quality of life was reported to increase during use (Gourgou-Bourgade et al. 2013).

Erlotinib, an epidermal growth factor receptor inhibitor, is the only targeted therapy approved in the US, in combination with gemcitabine, for the first-line treatment of patients with locally advanced, unresectable or metastatic pancreatic cancer. The randomized controlled trial comparing erlotinib with placebo showed a 0.4-month median OS benefit and a median PFS benefit. BNT141, targeting the CLDN-18.2+ subpopulation of PDAC, could potentially address a population with significantly high unmet medical need. The sponsor aims to accelerate the clinical development of BNT141 in this indication by establishing a safe dose to be carried forward with the SOC (chemotherapy) during the first-in-human study.

### C. Biliary Tract Cancers

Biliary tracts cancers constitute epithelial malignancies of the biliary tree and include the following: gallbladder cancer, ampulla of Vater cancer, (the extra-hepatic and intra-hepatic bile ducts). Historically, the term encompasses extra hepatic and intra hepatic bile ducts, excluding gallbladder cancer and ampulla of Vater cancer (de Groen et al. 1999).

Biliary tracts cancers constitute approximately 3% of all gastrointestinal malignancies (Charbel et al. 2011) and is the most common hepatobiliary cancer after hepatocellular carcinoma (Hennedige et al. 2014). Unfortunately, the mortality rate (3.58 per 100,000) is very high. This is comparable to the incidence rate (3.64 per 100,000) in England (National Cancer Intelligence Network 2015) and equates to a 5-year survival of 2% in the metastatic setting (National Cancer Institute Seer Data 2015; Seer Data 2014). The global prevalence of BTC has risen by 22%, and 150,000 patients were diagnosed with BTC in 2015 (Vos et al. 2015). Overall, there is a large variation in incidence with certain areas depicting high prevalence (e.g., Japan and South Korea). This can be accounted for by liver fluke (Opisthorchis viverrini and Clonorchiasis sinensis) infestation in zones (north-east Thailand and China), where cholangiocarcinoma is more common (Parkin et al. 1991; Kahn et al. 2008). Areas with high prevalence of cholelithiasis correspond to a high prevalence of gallbladder cancer, such as India and Chile (Randi et al. 2009; Khan et al. 1999; Kirstein and Vogel. 2016). Geographical regions where the above mentioned risk factors are uncommon have fewer cases of BTC (Kahn et al. 1999).

Apart from the risk factors mentioned above, primary sclerosing cholangitis, primary biliary cirrhosis, cirrhosis due to other causes, hepatitis C and congenital malformations such as choledochal cysts and multiple biliary papillomatosis are also associated with an increased risk of developing BTC (Kahn et al. 2008; Lee et al. 2004; Chapman 1999). In addition, patients with germline mutations resulting in Lynch syndrome and BRCA1 and BRCA2 (breast cancer gene 1 and 2) genetic aberrations are also predisposed to BTC. There is a lifetime risk of 2% of developing BTC with Lynch syndrome and a relative risk of 4.97% of developing cholangiocarcinoma in carriers of BRCA2 (Golan et al. 2017; Shigeyasu et al. 2014).

Treatments for BTC are stratified according to the stage of the disease, where surgery remains the mainstay of cure in early stages, although this represents a small minority of patients (10-40%) (Cidon 2016). For the first-line treatment of advanced disease, the Phase 3 trial ABC-02 confirmed the superiority of the combination of gemcitabine and cisplatin over single-agent gemcitabine. Reported median OS was 11.7 months vs 8.1 months, respectively (hazard ratio [HR] 0.64; 95% confidence interval [CI] 0.52-0.80; p<0.001) (Valle et al. 2010), and since then this has become a global standard of care for late-stage BTC. Although the modest survival benefit gained from this regimen has not yet been surpassed in a randomized Phase 3 trial, the combination of gemcitabine with an oral fluoropyrimidine S-1, in a Phase 3 trial, reported a median OS of 15.1 months for the gemcitabine and S-1 arm vs 13.4 months in the gemcitabine/cisplatin arm (HR 0.95; 90% CI 0.78-1.15; p=0.046 for non-inferiority) (Morizane et al. 2018). This regimen may be considered as an alternative treatment for patients where comorbidities restrict the use of platinum agents. A Phase 2 trial evaluating the combination of gemcitabine, cisplatin and nab-paclitaxel in the first-line setting in patients with advanced BTC has reported a superior median PFS than that associated historically with the standard gemcitabine/cisplatin regimen (11.4 months versus 8.0 months) in the preliminary results with a median OS of 19.2 months. This trial (NCT02392637) was ongoing in 2019 (Shroff et al. 2017; Shroff et al. 2018).

### VII. Patient Populations

Technologies provided herein can be useful for treatment of diseases or conditions associated with elevated expression and/or activity of CLDN-18.2. In some embodiments, technologies provided herein can be useful for treatment of CLDN-18.2 positive solid tumors. In some embodiments, CLDN-18.2 positive solid tumors are determined by immunohistochemical analysis with a staining intensity score of 2 or higher in accordance with the practice of skilled pathologists.

The present disclosure, among other things, recognizes that pancreatic cancers and biliary cancers typically have high expression of CLDN-18.2 Accordingly, in some embodiments, technologies provided herein can be useful for treatment of pancreatic cancers. For example, in some embodiments, technologies provided herein can be useful for treatment of pancreatic ductal adenocarcinoma (PDAC). In some embodiments, technologies provided herein can be useful for treatment of biliary cancers.

In some embodiments, technologies provided herein can be useful for treatment of gastroesophageal cancer that are determined to be CLDN-18.2 positive, e.g., by immunohistochemical analysis. In some embodiments, technologies provided herein can be useful for treatment of non-small cell lung cancer (NSCLC) that are determined to be CLDN-18.2 positive, e.g., by immunohistochemical analysis.

In some embodiments, technologies provided herein can be useful for treatment of patients (e.g., adult patients) with CLDN-18.2+ solid tumors that are metastatic. In some embodiments, technologies provided herein can be useful for treatment of patients (e.g., adult patients) with CLDN-18.2+ solid tumors that are unresectable, e.g., in some embodiments where surgical resection is likely to result in severe morbidity. In some embodiments, technologies provided herein can be useful for treatment of patients (e.g., adult patients) with CLDN-18.2+ solid tumors that are locally advanced. Additionally or alternatively, in some embodiments, cancer in such patients may have progressed following treatment or such cancer patients may have no satisfactory alternative therapy.

In some embodiments, technologies provided herein can be useful for treatment of adult patients with locally advanced, unresectable or metastatic CLDN-18.2+ pancreatic cancer. In some embodiments, technologies provided herein can be useful for treatment of adult patients with locally advanced, unresectable or metastatic CLDN-18.2+ biliary tract cancer. In some embodiments, patients who are receiving a treatment described herein may have received other cancer therapy, e.g., but not limited to chemotherapy.

In some embodiments, a subject suffering from a CLDN-18.2 positive solid tumor may have received a pre-treatment sufficient to increase CLDN-18.2 level/activity such that his/her solid tumor is characterized as a CLDN-18.2-positive solid tumor (e.g., ones described herein). For example, in some embodiments, such a cancer patient may have received chemotherapy that is expected or predicted to elevate expression and/or activity of CLDN-18.2, or may result or have resulted in expression and/or activity of CLDN-18.2. For example, in some embodiments, such chemotherapy may be expected or predicted to elevate expression and/or activity of CLDN-18.2, or may result or have resulted in expression and/or activity of CLDN-18 by at least 50% or more, including, e.g., at least 60%, at least 70%, at least 80%, at least 90%, at least 95%, or higher, as compared to expression and/or activity of CLDN-18.2 in the absence of such chemotherapy. In some embodiments, such chemotherapy may be expected or predicted to elevate expression and/or activity of CLDN-18.2, or may result or have resulted in expression and/or activity of CLDN-18 by at least 2-fold or more, including, e.g., at least 2.5-fold, at least 3-fold, at least 3.5-fold, at least 4-fold, at least 4.5-fold, at least 5-fold, at least 5.5-fold, at least 6-fold, at least 7-fold, at least 8-fold, at least 9-fold, at least 10-fold, or higher, as compared to expression and/or activity of CLDN-18.2 in the absence of such chemotherapy. Examples of such chemotherapeutic agents include, but are not limited to nab-paclitaxel, gemcitabine, cisplatin, and/or FOLFIRINOX.

In some embodiments, a cancer patient who meets one or more of the disease-specific inclusion criteria as described in Example 16 are amenable to treatment described herein (e.g., receiving a provided pharmaceutical composition as monotherapy or as part of a combination therapy). In some embodiments, such a cancer patient that is administered a treatment described herein may further meets one or more of the other inclusive criteria as described in Example 16.

In some embodiments, a cancer patient who meets one or more of the disease-specific inclusion criteria as described in Example 16 are amenable to treatment described herein (e.g., receiving a provided pharmaceutical composition as monotherapy or as part of a combination therapy). In some embodiments, such a cancer patient that is administered a treatment described herein may further meets one or more of the other inclusive criteria as described in Example 16.

In some embodiments, a cancer patient whose tumor does not express CLDN-18.2 or is determined to be not CLDN-18.2 positive (e.g., in accordance with the present disclosure described herein) is not administered a treatment described herein.

In some embodiments, a cancer patient who has a CLDN-18.2 positive tumor but meets one or more of the exclusion criteria as described in Example 17 is not administered a treatment described herein.

### VIII. Treatment (e.g., Dosing Regimens)

In some embodiments, pharmaceutical compositions described herein can be taken up by target cells for production of an encoded CLDN-18.2-targeting antibody agent at therapeutically relevant plasma concentrations. In some embodiments, such pharmaceutical compositions described herein can deliver an encoded CLDN-18.2-targeting antibody agent at a plasma concentration that is sufficient to induce antibody-dependent cellular cytotoxicity (ADCC) and complement-dependent cytotoxicity (CDC) against target cells (e.g., tumor cells).

Accordingly, another aspect of the present disclosure relates to methods of using pharmaceutical compositions described herein. For example, one aspect provided herein is a method comprising administering a provided pharmaceutical composition to a subject suffering from a CLDN-18.2-positive solid tumor. In some embodiments, a provided pharmaceutical composition is administered by intravenous injection or infusion. Examples of a CLDN-18.2-positive solid tumor include but are not limited to a biliary tract tumor, a gastric tumor, a gastro-esophageal tumor, an ovarian tumor, a pancreatic tumor, and a tumor that expresses or exhibits a level of a CLDN-18.2 polypeptide above a threshold level (e.g., a CLDN-18.2 level as observed in normal tissues), for example, in some embodiments by at least 50% or more, including, e.g., at least 60%, at least 70%, at least 80%, at least 90%, at least 95%, or higher, or in some embodiments by at least 2-fold or more, including, e.g., at least 2.5-fold, at least 3-fold, at least 3.5-fold, at least 4-fold, at least 4.5-fold, at least 5-fold, at least 5.5-fold, at least 6-fold, at least 7-fold, at least 8-fold, at least 9-fold, at least 10-fold, or higher.

Another aspect of the present disclosure relates to certain improvement in a method of delivering a CLDN-18.2-targeting antibody agent for cancer treatment in a subject, which method comprises administering to a cancer subject a provided pharmaceutical composition. In some embodiments, pharmaceutical compositions described herein may achieve one or more improvements such as effective administration with reduced incidence (e.g., frequency and/or severity) of TEAEs, and/or with improved relationship between efficacy level and TEAE level (e.g., improved therapeutic window) relative to those observed when a corresponding (e.g., encoded) protein (e.g., antibody) agent itself is administered. In particular, the present disclosure teaches that such improvements in particular may be achieved by delivering IMAB362 via administration of a nucleic acid, and in particular of RNA(s) (e.g., ssRNA(s) such as mRNA(s))) encoding it.

Dosing schedule: Those skilled in the art are aware that cancer therapeutics often administered in dosing cycles. In some embodiments, pharmaceutical compositions described herein are administered in one or more dosing cycles.

In some embodiments, one dosing cycle is at least 3 or more days (including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 days. In some embodiments, one dosing cycle is at least 21 days.

In some embodiments, one dosing cycle may involve multiple doses, e.g., according to a pattern such as, for example, a dose may be administered daily within a cycle, or a dose may be administered every 2 days, every 3 days, every 4 days, every 5 days, every 6 days, every 7 days within a cycle.

In some embodiments, multiple cycles may be administered. For example, in some embodiments, at least 2 cycles (including, e.g., at least 3 cycles, at least 4 cycles, at least 5 cycles, at least 6 cycles, at least 7 cycles, at least 8 cycles, at least 9 cycles, at least 10 cycles, or more) can be administered. In some embodiments, the number of dosing cycles to be administered may vary with types of treatment (e.g., monotherapy vs. combination therapy). In some embodiments, at least 3-8 dosing cycles may be administered.

In some embodiments, there may be a “rest period” between cycles; in some embodiments, there may be no rest period between cycles. In some embodiments, there may be sometimes a rest period and sometimes no rest period between cycles.

In some embodiments, a rest period may have a length within a range of several days to several months. For example, in some embodiments, a rest period may have a length of at least 3 days or more, including, e.g., at least 4 days, at least 5 days, at least 6 days, at least 7 days, at least 8 days, at least 9 days, at least 10 days, at least 11 days, at least 12 days, at least 13 days, at least 14 days or more. In some embodiments, a rest period may have a length of at least 1 week or more, including, e.g., at least 2 weeks, at least 3 weeks, at least 4 weeks, or more.

In some embodiments, pharmaceutical compositions described herein, for example, for use in monotherapy, can be administered in at least three cycles, wherein in some embodiments each cycle is 21 days. In some embodiments, pharmaceutical compositions described herein, for example, for use in combination therapy, can be administered in at least eight cycles, wherein in some embodiments each cycle is 21 days.

In some embodiments, a pharmaceutical composition provided herein can be administered on Day 1 of each 3-week dosing cycle (21 days/Q3W). In some embodiments, a cancer patient suffering from a CLDN-18.2+ solid tumor can receive a maximum of three cycles of treatment. In some embodiments, a cancer patient suffering from a CLDN-18.2+ solid tumor can receive a maximum of eight cycles.

Dose: Dosage of pharmaceutical compositions described herein may vary with a number of factors including, e.g., but not limited to body weight of a subject to be treated, cancer types and/or cancer stages, and/or monotherapy or combination therapy. In some embodiments, a dosing cycle involves administration of a set number and/or pattern of doses. For example, in some embodiments, a pharmaceutical composition described herein is administered at least one dose per dosing cycle, including, e.g., at least two doses per dosing cycle, at least three doses per dosing cycle, at least four doses per dosing cycle, or more.

In some embodiments, a dosing cycle involves administration of a set cumulative dose, e.g., over a particular period of time, and optionally via multiple doses, which may be administered, for example, at set interval(s) and/or according to a set pattern. In some embodiments, a set cumulative dose may be administered via multiple doses at set intervals such that there is at least some temporal overlap in biological and/or pharmacokinetics effects generated by such multiple doses on a target cell or on a subject being treated. In some embodiments, a set cumulative dose may be administered via multiple doses at set intervals such that biological and/or pharmacokinetics effects generated by such multiple doses on a target cell or on a subject being treated may be additive. By way of example only, in some embodiments, a set cumulative dose of X mg may be administered via two doses with each dose of X/2 mg, wherein such two doses are administered sufficiently close in time such that biological and/or pharmacokinetics effects generated by each X/2-mg dose on a target cell or on a subject being treated may be additive.

In some embodiments, each dose or a cumulative dose (e.g., for intravenous administration) is administered at a level such that a CLDN-18.2-targeting antibody agent expressed from provided single-stranded RNA(s) is expected to achieve level (e.g., plasma level and/or tissue level) that is high enough to trigger antibody-dependent cellular cytotoxicity against target cells (e.g., cancer cells) throughout a dosing cycle. For IMAB362, the dose-response correlation for ADCC is clinically well characterized and efficient lysis of CLDN-18.2+ cells through ADCC with an EC₉₅ of 0.3-28 μg/mL has been reported (Sahin et al. 2018). Thus, in some embodiments, each dose or a cumulative dose (e.g., for intravenous administration) is administered in an amount that confers a plasma concentration of about 0.3-28 μg/mL of a CLDN-18.2-targeting antibody agent encoded by ssRNA(s) (e.g., ones described herein).

In some embodiments, each dose or a cumulative dose (e.g., for intravenous administration) is administered at a level such that a CLDN-18.2-targeting antibody agent expressed from provided single-stranded RNA(s) is expected to achieve level (e.g., plasma level and/or tissue level) comparable to the therapeutically relevant level (e.g., plasma level and/or tissue level) observed with administration of IMAB362. In some embodiments, each dose or a cumulative dose (e.g., for intravenous administration) is administered at a level such that a CLDN-18.2-targeting antibody agent expressed from provided single-stranded RNA(s) is expected to achieve level (e.g., plasma level and/or tissue level) above about 0.05-3 μg/mL; in some embodiments, above about 0.1-10 μg/mL; in some embodiments above about 0.2-15 μg/mL; in some embodiments, above about 0.3-30 μg/mL; in some embodiments, above about 0.3-28 μg/mL. In some embodiments, each dose or a cumulative dose (e.g., for intravenous administration) is administered at a level such that a CLDN-18.2-targeting antibody agent expressed from provided single-stranded RNA(s) is expected to achieve C_{trough} level (e.g., plasma level and/or tissue level) above about 5 μg/mL; in some embodiments above about 10 μg/mL; in some embodiments above about 15 μg/mL.

In some embodiments, each dose or a cumulative dose (e.g., for intravenous administration) is administered to deliver one or more ssRNAs described herein (e.g., mRNA) encoding a CLDN-18.2-targeting antibody agent at a level expected to achieve level (e.g., plasma level and/or tissue level) of such an antibody above about 0.1 μg/mL; in some embodiments, above about 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2 μg/mL, 5 μg/mL, 8 μg/mL, 10 μg/mL, 15 μg/mL, 20 μg/mL, 25 μg/mL, or have a range up to and above what is observed with IMAB362 antibody administration.

Without wishing to be bound by any particular theory, the present disclosure provides an insight that AUC of IMAB362 may not accurately elucidate a concentration that is pharmacologically active over a dosing cycle (e.g., over a 21-day dosing cycle) when applied to an mRNA encoded antibody. In some embodiments, AUC is monitored or measured at least once. In some embodiments, AUC is not monitored or measured. Regardless, in many embodiments, a dosing amount and/or frequency may be independent of AUC of IMAB362.

Without wishing to be bound by any particular theory, the present disclosure, among other things, provides an insight that reaching the Cₘₐₓ reported for IMAB362 may not be necessary and may increase the risk of toxicities induced by pharmaceutical compositions described herein and the respective antibody agent expressed therefrom. For example, in some embodiments, pharmaceutical compositions described herein can have an improved pharmacokinetics profile that keeps a biological active dose of the antibody over a prolonged period of time due to continued expression from the RNA. Accordingly, in some embodiments, pharmaceutical compositions described herein may be dosed at a level such that a RiboMab targeting CLDN-18.2 that is expressed from provided single-stranded RNA(s) is expected to achieve level (e.g., plasma level and/or tissue level) below Cₘₐₓ reported for IMAB362. In some embodiments, a dosing amount and/or frequency may be independent of Cₘₐₓ reported for IMAB362.

In some embodiments, each dose or a cumulative dose of a pharmaceutical composition described herein (e.g., for intravenous administration) may comprise one or more ssRNAs encoding a CLDN-18.2-targeting antibody agent (whether encoded by a single ssRNA or two or more ssRNAs) in an amount within a range of 0.1 mg RNA/kg to 5 mg RNA/kg body weight of a subject to be administered. In some embodiments, each dose or a cumulative dose may comprise ssRNA(s) (e.g., ones described herein) in an amount of 0.1 mg RNA/kg, 0.15 mg RNA/kg, 0.2 mg RNA/kg, 0.225 mg RNA/kg, 0.25 mg RNA/kg, 0.3 mg RNA/kg, 0.35 mg RNA/kg, 0.4 mg RNA/kg, 0.45 mg RNA/kg, 0.5 mg RNA/kg, 0.55 mg RNA/kg, 0.6 mg RNA/kg, 0.65 mg RNA/kg, 0.7 mg RNA/kg, 0.75 mg RNA/kg, 0.80 mg RNA/kg, 0.85 mg RNA/kg, 0.9 mg RNA/kg, 0.95 mg RNA/kg, 1.0 mg RNA/kg, 1.25 mg RNA/kg, 1.5 mg RNA/kg, 1.75 mg RNA/kg, 2.0 mg RNA/kg, 2.25 mg RNA/kg, 2.5 mg RNA/kg, 2.75 mg RNA/kg, 3.0 mg RNA/kg, 3.25 mg RNA/kg, 3.5 mg RNA/kg, 4 mg RNA/kg, 5 mg RNA/kg, or higher. In some embodiments, each dose or a cumulative dose may comprise ssRNA(s) (e.g., ones described herein) in an amount of 1.5 mg RNA/kg. In some embodiments, each dose or a cumulative dose may comprise ssRNA(s) (e.g., ones described herein) in an amount of 5 mg RNA/kg.

In some embodiments, each dose or a cumulative dose of a provided pharmaceutical composition (e.g., for intravenous administration) is administered to deliver a dose of 0.15 mg RNA/kg, which in some embodiments may correspond to approximately 7 μg/mL CLDN-18.2-targeting antibody agent at Cmax. FIG. 14 shows the dose-exposure correlation of RNA drug substance encoding CLDN-18.2-targeting antibody agent in cynomolgus monkey at tmax (48 hours). As will be appreciated by a skilled artisan, assuming that LNP-transfection efficacy and mRNA translation is comparable between cynomolgus monkey and humans (Coelho et al. 2013), in some embodiments, each dose or a cumulative dose of a provided pharmaceutical composition (e.g., for intravenous administration) may be administered to deliver an appropriate dose corresponding to desirable plasma level of CLDN-18.2-targeting antibody agent encoded by ssRNA(s) as shown in FIG. 14.

In some embodiments, dosing may be adjusted based on response of a subject receiving the therapy. For example, in some embodiments, dosing may involve administration of a higher dose followed later by administration of a lower dose if one or more parameters for safety pharmacology assessment (e.g., as described in Example 5) indicates that the prior dose may not satisfy the medical safety requirement according to a physician. In some embodiments, dose escalation may be performed at one or more of the levels shown in Table 13 of Example 8; in some embodiments, dose escalation may involve administration of at least one lower dose from Table 13 followed later by administration of at least one higher dose from Table 13. Without wishing to be bound by any particular theory, the present disclosure, among other things, provides an insight that a pharmaceutically guided dose escalation (PGDE) method may be applied to determine an appropriate dose of pharmaceutical compositions described herein. An exemplary dose escalation study is provided in Example 8.

Also provided herein is also a method of determining a dosing regimen of a pharmaceutical composition targeting CLDN-18.2. For example, in some embodiments, such a method comprises steps of: (A) administering a pharmaceutical composition (e.g., ones described herein) to a subject suffering from a CLDN-18.2 positive solid tumor under a pre-determined dosing regimen; (B) monitoring or measuring tumor size of the subject periodically over a period of time; (C) evaluating the dosing regimen based on the tumor size measurement(s). For example, a dose and/or dosage frequency can be increased if reduction in tumor size after the administration of a pharmaceutical composition (e.g., ones described herein) is not therapeutically relevant; or a dose and/or dosage frequency can be decreased if reduction in tumor size after the administration of a pharmaceutical composition (e.g., ones described herein) is therapeutically relevant, but adverse effect (e.g., toxicity effect) is shown in the subject. If reduction in tumor size after the administration of a pharmaceutical composition (e.g., ones described herein) is therapeutically relevant, and no adverse effect (e.g., toxicity effect) is shown in the subject, no changes is made to a dosage regimen.

In some embodiments, such a method of determining a dosing regimen of a pharmaceutical composition targeting CLDN-18.2 may be performed in a group of animal subjects (e.g., mammalian non-human subjects) each a bearing a human CLDN-18.2 positive xenograft tumor. In some such embodiments, a dose and/or dosage frequency can be increased if less than 30% of the animal subjects exhibit reduction in tumor size after the administration of a pharmaceutical composition (e.g., ones described herein) and/or extent of reduction in tumor size exhibited by the animal subjects is not therapeutically relevant; or a dose and/or dosage frequency can be decreased if reduction in tumor size after the administration of a pharmaceutical composition (e.g., ones described herein) is therapeutically relevant, but significant adverse effect (e.g., toxicity effect) is shown in at least 30% of the animal subjects. If reduction in tumor size after the administration of a pharmaceutical composition (e.g., ones described herein) is therapeutically relevant, and no significant adverse effect (e.g., toxicity effect) is shown in the animal subjects, no changes is made to a dosage regimen.

Although the dosing regimens (e.g., dosing schedule and/or doses) provided herein are principally suitable for administration to humans, it will be understood by the skilled artisan that dose equivalents can be determined for administration to animals of all sorts. The ordinarily skilled veterinary pharmacologist can design and/or perform such determination with merely ordinary, if any, experimentation.

In some embodiments, pharmaceutical compositions described herein can be administered patients with CLDN-18.2+ solid tumors as monotherapy.

Combination therapy: The present disclosure, among other things, provides an insight that the capability of pharmaceutical compositions targeting CLDN-18.2 as described herein to induce antibody-dependent cellular cytotoxicity (ADCC) and/or complement-dependent cytotoxicity (CDC) against target cells (e.g., tumor cells) while leveraging immune system of recipient subjects can augment cytotoxic effect(s) of chemotherapy and/or other anti-cancer therapy. In some embodiments, such a combination therapy may prolong progression-free and/or overall survival, e.g., relative to individual therapies administered alone and/or to another appropriate reference. Accordingly, in some embodiments, pharmaceutical compositions described herein can be administered in combination with other anti-cancer agents in patients with CLDN-18.2+ solid tumors.

Without wishing to be bound by a particular theory, the present disclosure observes that certain chemotherapeutic agents, for example such as gemcitabine, oxaliplatin, and 5-fluorouracil were shown to upregulate existing CLDN-18.2 expression levels in pancreatic cancer cell lines; moreover, these agents were not observed to increase de novo expression in CLDN-18.2—negative cell lines. See, e.g., Tureci et al. (2019) “Characterization of Zolbetuximab in pancreatic cancer models” In Oncoimmunology 8 (1), pp. e1523096.

The present disclosure, among other things, provides an insight that CLDN-18.2-targeted therapy as described herein may be particularly useful and/or effective when administered to tumor(s) (e.g., tumor cells, subjects in whom such tumor(s) and/or tumor cell(s) are suspected and/or have been detected, etc.) characterized by (e.g., that have been determined to display and/or that are expected or predicted to display) elevated expression and/or activity of CLDN-18.2 expression in tumor cells (e.g., as may result or have resulted from exposure to one or more chemotherapeutic agents). Indeed, among other things, the present disclosure teaches that provided CLDN-18.2-targeted therapy (e.g., administration of a nucleic acid such as an RNA and, more particularly an mRNA encoding a CLDN-18.2-targeting antibody agent) as described herein may provide synergistic therapeutic when administered in combination with (e.g., to a subject who has received and/or is receiving or has otherwise been exposed to) one or more CDLN-18.2-enhancing agents (e.g., one or more certain chemotherapeutic agents). Accordingly, in some embodiments, CLDN-18.2-targeted therapy as described herein can be useful in combination with other anti-cancer agents that are expected to and/or have been demonstrated to up-regulate CLDN-18.2 expression and/or activity in tumor cells. For example, in some embodiments, pharmaceutical compositions described herein may be combined with an already efficient but not durable cytotoxic treatment.

In some embodiments, a provided pharmaceutical composition may be administered as part of combination therapy comprising such a pharmaceutical composition and a chemotherapeutic agent. Accordingly, in some embodiments, a provided pharmaceutical composition may be administered to a subject suffering from a CLDN-18.2+ solid tumor who has received a chemotherapeutic agent. In some embodiments, a provided pharmaceutical composition may be co-administered with a chemotherapeutic agent to a subject suffering from a CLDN-18.2+ solid tumor. In some embodiments, a provided pharmaceutical composition and a chemotherapeutic agent may be administered concurrently or sequentially. For example, in some embodiments, a first dose of chemotherapeutic agent may be administered after (e.g., at least four hours after) administration of a provided pharmaceutical composition. In some embodiments, a chemotherapeutic agent and a provided pharmaceutical composition are concomitantly administered.

In some embodiments where a chemotherapeutic agent is expected to elevate expression and/or activity of CLDN-18.2 in a cancer subject, such a chemotherapeutic agent can be administered prior to administration of a provided pharmaceutical composition. In some embodiments, a pharmaceutical composition described herein can be administered at a time such that a CLDN-18.2-targeting antibody agent expressed from ssRNA(s) described herein reaches its therapeutically relevant plasma concentration (e.g., as described herein) during elevation in expression and/or activity of CLDN-18.2 in response to administration of such a chemotherapeutic agent. In some embodiments, a pharmaceutical composition described herein can be administered at a time such that a CLDN-18.2-targeting antibody agent expressed from ssRNA(s) described herein reaches its therapeutically relevant plasma concentration (e.g., as described herein) while expression and/or activity of CLDN-18.2 is elevated in response to such a chemotherapeutic agent by at least 50% or more, including, e.g., at least 60%, at least 70%, at least 80%, at least 90%, at least 95%, or higher, as compared to expression and/or activity of CLDN-18.2 in the absence of such a chemotherapeutic agent. In some embodiments, a pharmaceutical composition described herein can be administered at a time such that a CLDN-18.2-targeting antibody agent expressed from ssRNA(s) described herein reaches its therapeutically relevant plasma concentration (e.g., as described herein) while expression and/or activity of CLDN-18.2 is elevated in response to such a chemotherapeutic agent by at least 1.5-fold, at least 2-fold or more, including, e.g., at least 2.5-fold, at least 3-fold, at least 3.5-fold, at least 4-fold, at least 4.5-fold, at least 5-fold, at least 5.5-fold, at least 6-fold, at least 7-fold, at least 8-fold, at least 9-fold, at least 10-fold, or higher, as compared to expression and/or activity of CLDN-18.2 in the absence of such a chemotherapeutic agent. Examples of such chemotherapeutic agents include, but are not limited to nab-paclitaxel, gemcitabine, cisplatin, and/or FOLFIRINOX.

Combination treatment with an anti-cancer therapy comprising gemcitabine: In some embodiments, an administered therapy comprising a provided pharmaceutical composition may be co-administered or overlap with an anti-cancer therapy comprising gemcitabine. Gemcitabine kills cells undergoing deoxyribonucleic acid (DNA) synthesis and blocks the progression of cells through the G1/S-phase boundary. Gemcitabine is metabolized by nucleoside kinases to diphosphate and triphosphate (dCTP) nucleosides. Gemcitabine diphosphate inhibits ribonucleotide reductase, an enzyme responsible for catalyzing the reactions that generate deoxynucleoside triphosphates for DNA synthesis, resulting in reductions in deoxynucleotide concentrations, including dCTP. Gemcitabine triphosphate competes with dCTP for incorporation into DNA. The reduction in the intracellular concentration of dCTP by the action of the diphosphate enhances the incorporation of gemcitabine triphosphate into DNA (self-potentiation). After the gemcitabine nucleotide is incorporated into DNA, only one additional nucleotide is added to the growing DNA strands, which eventually results in the initiation of apoptotic cell death.

Combination treatment with an anti-cancer therapy comprising nab paclitaxel: In some embodiments, an administered therapy comprising a provided pharmaceutical composition may be co-administered or overlap with an anti-cancer therapy comprising nab-paclitaxel. Nab-paclitaxel is an albumin-bound form of paclitaxel with a mean particle size of approximately 130 nm. It is a microtubule inhibitor that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. Paclitaxel induces abnormal arrays or ‘bundles’ of microtubules throughout the cell cycle and multiple asters of microtubules during mitosis.

Combination treatment with an anti-cancer therapy comprising cisplatin: In some embodiments, an administered therapy comprising a provided pharmaceutical composition may be co-administered or overlap with an anti-cancer therapy comprising cisplatin. Cisplatin is a heavy metal complex containing a central atom of platinum surrounded by two chloride atoms and two ammonia molecules in the cis position. Without wishing to be bound by theory, cisplatin is believed to kill cancer cells by binding to DNA and interfering with its repair mechanism, eventually leading to cell death.

Combination treatment with an anti-cancer therapy comprising FOLFIRINOX: In some embodiments, an administered therapy comprising a provided pharmaceutical composition may be co-administered or overlap with an anti-cancer therapy comprising FOLFIRINOX, which is a combination of cancer drugs that includes: FOL-folinic acid (also called leucovorin, calcium folinate, or FA); F-fluorouracil (also called 5FU); Irin-irinotecan; Ox-oxaliplatin.

Leucovorin is a mixture of the diastereoisomers of the 5-formyl derivative of tetrahydrofolic acid. The biologically active compound of the mixture is the (−)-1-isomer, known as citrovorum factor or (−)-folinic acid. Leucovorin does not require reduction by the enzyme dihydrofolate reductase in order to participate in reactions utilizing folates as a source of “one-carbon” moieties. 1-Leucovorin (1-5-formyltetrahydrofolate) is rapidly metabolized (via 5, 10-methenyltetrahydrofolate then 5, 10-methylenetetrahydrofolate) to 1,5 methyltetrahydrofolate. 1,5-Methyltetrahydrofolate can in turn be metabolized via other pathways back to 5,10-methylenetetrahydrofolate, which is converted to 5-methyltetrahydrofolate by an irreversible, enzyme catalyzed reduction using the cofactors flavin adenine dinucleotide and nicotinamide-adenine dinucleotide phosphate.

Leucovorin can enhance the therapeutic and toxic effects of fluoropyrimidines used in cancer therapy, such as 5-fluorouracil. Concurrent administration of leucovorin does not appear to alter the plasma PK of 5-fluorouracil. 5-Fluorouracil is metabolized to fluorodeoxyuridylic acid, which binds to and inhibits the enzyme thymidylate synthase (an enzyme important in DNA repair and replication). Leucovorin is readily converted to another reduced folate, 5,10-methylenetetrahydrofolate, which acts to stabilize the binding of fluorodeoxyridylic acid to thymidylate synthase and thereby enhances the inhibition of this enzyme.

Fluorouracil is a nucleoside metabolic inhibitor that interferes with the synthesis of DNA and to a lesser extent inhibits the formation of RNA; these affect rapidly growing cells and may lead to cell death. Fluorouracil is converted to three main active metabolites: 5-fluoro-2′-deoxyuridine-5′-monophosphate, 5-fluorouridine-5′ triphosphate and 5-fluoro-2′-deoxyuridine-5′-triphosphate. These metabolites have several effects including the inhibition of thymidylate synthase by 5-fluoro-2′-deoxyuridine-5′-monophosphate, incorporation of 5-fluorouridine-5′ triphosphate into RNA and incorporation of 5-fluoro-2′-deoxyuridine-5′-triphosphate into DNA.

Irinotecan is a derivative of camptothecin. Camptothecins interact specifically with the enzyme topoisomerase I, which relieves torsional strain in DNA by inducing reversible single-strand breaks. Irinotecan and its active metabolite SN-38 bind to the topoisomerase I-DNA complex and prevent religation of these single-strand breaks. Current research suggests that the cytotoxicity of irinotecan is due to double-strand DNA damage produced during DNA synthesis when replication enzymes interact with the ternary complex formed by topoisomerase I, DNA, and either irinotecan or SN-38. Mammalian cells cannot efficiently repair these double-strand breaks.

Oxaliplatin undergoes non-enzymatic conversion in physiologic solutions to active derivatives via displacement of the labile oxalate ligand. Several transient reactive species are formed, including monoaquo and diaquo DACH platinum, which covalently bind with macromolecules. Both inter and intrastrand plasma tumor DNA crosslinks are formed. Crosslinks are formed between the N7 positions of two adjacent guanines, adjacent adenine-guanines, and guanines separated by an intervening nucleotide. These crosslinks inhibit DNA replication and transcription. Cytotoxicity is cell-cycle nonspecific.

In some embodiments, technologies provided herein are useful for administration to a subject suffering from a CLDN-18.2 positive pancreatic tumor. In some embodiments, such a subject may be receiving a provided pharmaceutical composition as a monotherapy or as part of a combination therapy comprising such a provided pharmaceutical composition and a chemotherapeutic agent indicated for treatment of pancreatic tumor. In some embodiments, such a chemotherapeutic agent may be or comprise FOLFIRINOX, which is a combination of cancer drugs including: folinic acid (FOL), fluorouracil (F), irinotecan (IRIN), and oxalipatin (OX). In some embodiments, such a chemotherapeutic agent may be or comprise gemcitabine and/or paclitaxel (e.g., nab-paclitaxel). In some embodiments, a pharmaceutical composition described herein can be administered in combination with gemcitabine according to the approved dose and treatment schedule of gemicitabine (e.g., Gemzar) as monotherapy for treatment of pancreatic cancer as described in Example 18. In some embodiments, a pharmaceutical composition described herein can be administered in combination with gemcitabine at a lower dose (e.g., less than 10%, less than 20%, less than 30%, or more) and/or under a less aggressive treatment schedule (e.g., every 10 days, or biweekly, etc.) than the approved dose and treatment schedule for gemicitabine (e.g., Gemzar) as monotherapy for treatment of pancreatic cancer as described above. In some embodiments, a pharmaceutical composition described herein can be administered in combination with gemcitabine and nab-paclitaxel according to the approved dose and treatment schedule of nab-paclitaxel/gemcitabine combination treatment as described in Example 18. In some embodiments, a provided pharmaceutical composition described herein can be administered in combination with nab-paclitaxel and gemcitabine, at least one of which is at a lower dose (e.g., less than 10%, less than 20%, less than 30%, or more) and/or under a less aggressive treatment schedule (e.g., every 10 days, or biweekly, etc.) than the approved dose and treatment schedule of nab-paclitaxel/gemcitabine combination treatment as described in Example 18. In some embodiments, a provided pharmaceutical composition described herein can be administered in combination with nab-paclitaxel and gemcitabine following the dosing schedule as described in Table 17 (Example 18).

In some embodiments, technologies provided herein are useful for administration to a subject suffering from a CLDN-18.2 positive biliary tract tumor. In some embodiments, such a subject may be receiving a provided composition as a monotherapy or as part of a combination therapy comprising such a provided pharmaceutical composition and a chemotherapeutic agent indicated for treatment of biliary tract tumor. In some embodiments, such a chemotherapeutic agent may be or comprise gemcitabine and/or cisplatin.

Efficacy monitoring: In some embodiments, patients receiving a provided treatment may be monitored periodically over the dosing regimen to assess efficacy of the administered treatment. For example, in some embodiments, efficacy of an administered treatment may be assessed by on-treatment imaging periodically, e.g., every 4 weeks, every 5 weeks, every 6 weeks, every 7 weeks, every 8 weeks, or longer. In some embodiments, one or more efficacy assessments as described in Example 19 may be performed.

In some embodiments, one or more of various pharmacokinetics and pharmacodynamics markers (e.g., as described in Example 6), which might act as anti-tumor and safety indicators of activity of provided pharmaceutical compositions (e.g., as monotherapy or as combination therapy, e.g., with standard of care, can be evaluated.

### EXEMPLIFICATION

### Example 1: In Vitro Characterization of a CLDN-18.2-Targeting Antibody Agent Expressed from One or More Exemplary mRNAs

The present Example demonstrates in vitro characterization of an exemplary CLDN-18.2-targeting antibody agent expressed from one or more mRNAs encoding the same upon introduction into cells.

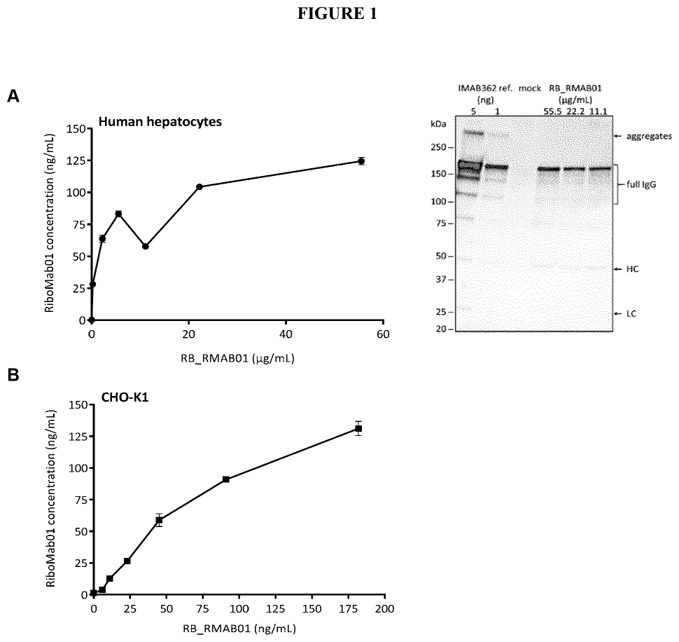

Assembly of full IgG after RNA transfection of hepatocytes. This Example shows translation, assembly and secretion of a CLDN-18.2-targeting antibody agent expressed from one or more exemplary mRNAs (e.g., ones described herein) (hereinafter “CLDN-18.2-targeting RiboMab”) after cellular uptake of the respective mRNAs in vitro. In this Example, two different expression systems, primary human hepatocytes to resemble liver targeting in vitro and Chinese hamster ovary cells (CHO-K1) were utilized. Lipofections of cells were performed with compositions comprising mRNAs encoding CLDN-18.2-targeting antibody agents described herein. Cell supernatants containing secreted CLDN-18.2-targeting RiboMab were harvested, for example, after 48 hours and analyzed, for example, via Western Blot and ELISA. Fully assembled CLDN-18.2-targeting RiboMab (e.g., CLDN-18.2-targeting IgG antibody) was generated in both expression systems (FIG. 1).

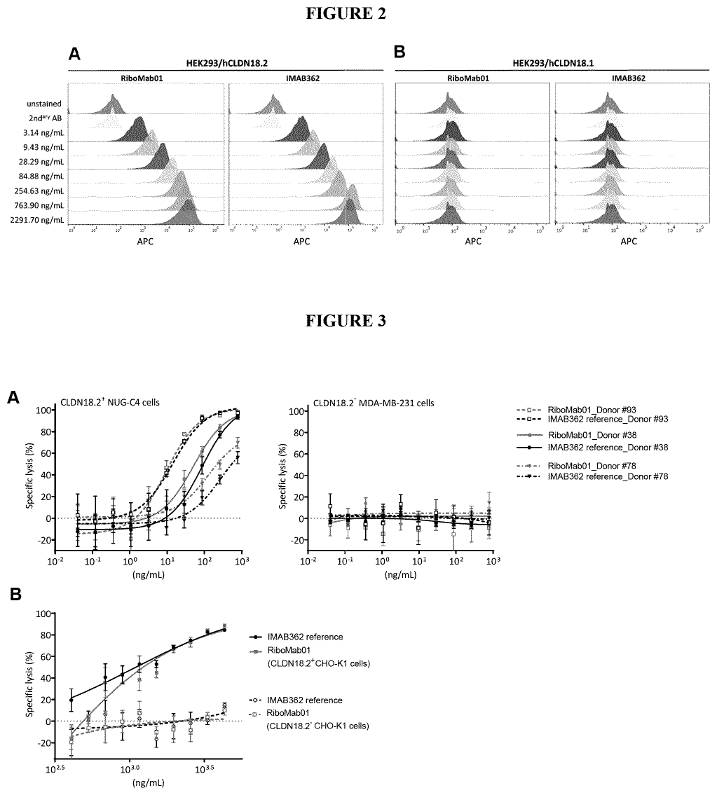

Binding specificity of an exemplary CLDN-18.2-targeting RiboMab. To determine the target specificity of an exemplary CLDN-18.2-targeting antibody agent expressed from one or more exemplary mRNAs (e.g., ones described herein) to a CLDN-18.2 polypeptide, flow cytometric binding assays were conducted using cell culture supernatant containing CLDN-18.2-targeting RiboMab expressed in CHO-K1 cells and CLDN-18.2+ HEK293 transfectants as target cells. To assess cross reactivity of CLDN-18.2-targeting RiboMab to the closely related splice variant CLDN18.1, binding of CLDN-18.2-targeting RiboMab to CLDN18.1 transfected cells was tested. CLDN-18.2-targeting RiboMab expressed from one or more exemplary mRNAs (e.g., ones described herein) bound preferentially to a tight junction polypeptide CLDN-18.2 polypeptide relative to a CLDN18.1 polypeptide. In some embodiments, the binding of CLDN-18.2-targeting RiboMab expressed from one or more exemplary mRNAs (e.g., ones described herein) was restricted or specific to CLDN-18.2 polypeptide and showed concentration dependency, comparable to the reference protein IMAB362 (or known as Zolbetuximab or Claudiximab) (FIG. 2).

Mode of action analysis: Antibody-dependent cellular cytotoxicity (ADCC) and complement-dependent cytotoxicity (CDC) in vitro. The bioactivity of CLDN-18.2-targeting RiboMab, expressed by CHO-K1 cells following in vitro translation from one or more mRNAs encoding the same (e.g., ones described herein), was assessed by analyzing antibody-dependent cellular cytotoxicity (ADCC) and complement-dependent cytotoxicity (CDC). Exemplary ADCC assays were conducted, for example, using the CLDN-18.2+ gastric carcinoma transfectants (e.g., NUG-C4) and the target-negative breast cancer cell line (e.g., MDA-MB-231) to assess specific lysis. For exemplary CDC assays, the CLDN-18.2+ transfectants (e.g., CHO-K1) and a CLDN-18.2-negative (e.g., CHO-K1) cell line were utilized. To simulate in vivo conditions, human PBMCs from three different healthy donors were used as effector cells in ADCC assays in an effector to target (E:T) ratio of 30:1 and human serum (e.g., commercially available human serum) was used as a source of complement in CDC assays. CLDN-18.2-targeting RiboMab efficiently mediated a target specific and dose-dependent cellular cytotoxicity comparable to the reference protein IMAB362 in ADCC [FIG. 3, Panel A; EC₅₀ 10-127 ng/mL (CLDN-18.2-targeting RiboMab), 14-265 ng/mL (IMAB362)] and CDC assays (FIG. 3, Panel B).

### Example 2: Characterization of a CLDN-18.2-Targeting Antibody Agent Expressed In Vivo from One or More Exemplary mRNAs in Rodents

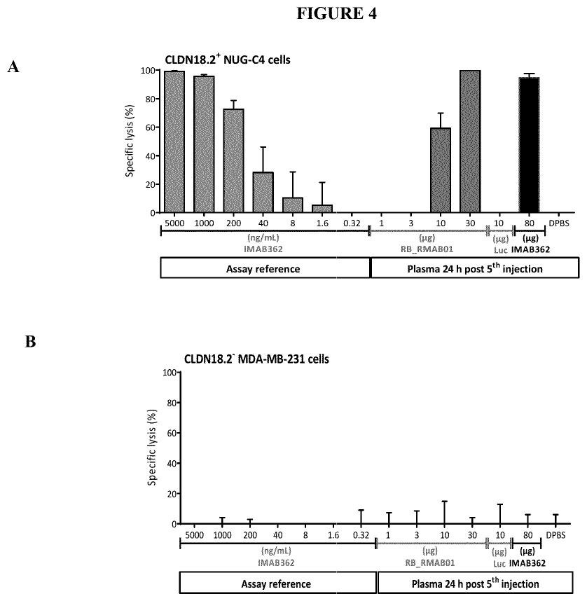

The bioactivity of CLDN-18.2-targeting RiboMab expressed in vivo from exemplary mRNAs (e.g., ones described herein) was assessed in ex vivo ADCC assays. ADCC assays were conducted using CLDN-18.2-targeting RiboMab or IMAB362-containing plasma of Balb/cJRj mice sampled 24 hours post 5^{th} IV dosing of 1 (˜0.04 mg/kg), 3 (˜0.10 mg/kg), 10 μg (˜0.40 mg/kg) and 30 μg (˜1.20 mg/kg) pharmaceutical composition comprising at least one or more mRNAs encoding a CLDN-18.2-targeting antibody agent (“CLDN-18.2-targeting RNA composition”) or 80 μg (˜3.20 mg/kg) IMAB362. Plasma of untreated mice spiked with IMAB362 served as assay reference. The CLDN-18.2+ gastric carcinoma transfectants (e.g., NUG-C4) were used as target and human PBMCs from healthy donors as effector cells. Target and effector cells were incubated for 48 hours in an E:T (effector to target) ratio of 30:1 with 1% of CLDN-18.2-targeting RiboMab-containing plasma and the ADCC was determined in a luciferase-based assay. CLDN-18.2-targeting RiboMab expressed in rodents exhibited a high and dose-dependent target cell lysis similar to 80 μg (˜3.20 mg/kg) of the reference protein IMAB362 (FIG. 4, Panel A). No unspecific lysis was seen on the target-negative breast cancer cell line MDA-MB-231 used as control indicating the target specificity of CLDN-18.2-targeting RiboMab (FIG. 4, Panel B). The results show that CLDN-18.2-targeting RiboMab expressed in rodents can mediate high ADCC of targeted tumor cells.

### Example 3: Characterization of a CLDN-18.2-Targeting Antibody Agent Expressed In Vivo from One or More Exemplary mRNAs in Non-Human Primates

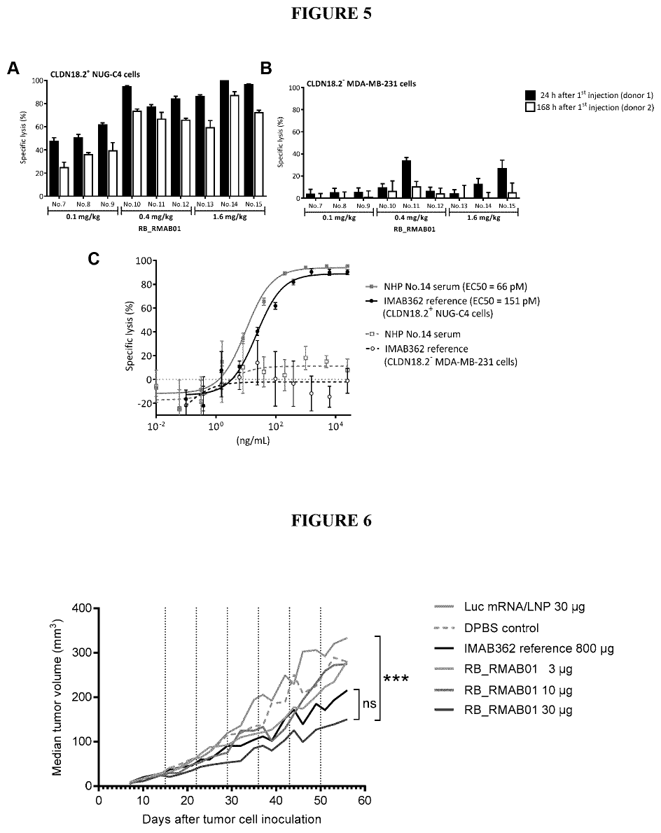

To determine the bioactivity of CLDN-18.2-targeting RiboMab in a phylogenetic and physiological closely related organism to humans, ADCC studies with CLDN-18.2-targeting RiboMab-containing serum of non-human primates (NHP), e.g., Cynomolgus monkey, sampled 24 hours and 168 hours post IV dosing of 0.1 mg/kg, 0.4 mg/kg and 1.6 mg/kg CLDN-18.2-targeting RNA composition were conducted. ADCC assays were performed as described in Example 2. CLDN-18.2-targeting RiboMab expressed in NHP exhibited a high and dose-dependent target cell lysis (FIG. 5, Panel A). Low effector, donor-dependent unspecific lysis was seen on the target-negative breast cancer cell line MDA-MB-231 (FIG. 5, Panel B). Serum of monkey No. 14, the animal with the highest determined CLDN-18.2-targeting RiboMab concentration (232 μg/mL), collected 48 hours after the 3^{rd} injection of CLDN-18.2-targeting RNA composition, was subjected to a luciferase-based ADCC assay in a 10-point dilution row. Purified IMAB362 served as assay reference protein. CLDN-18.2-targeting RiboMab expressed by NHP mediated a high and specific lysis of NUG-C4 target cells (FIG. 5, Panel C) with an EC₅₀ of 10 ng/mL (66 pM). These results show that CLDN-18.2-targeting RiboMab expressed by NHP mediates can mediate potent and target specific ADCC.

### Example 4: Intravenously Administered CLDN-18.2-Targeting RNA Composition Mediates Tumor Growth Inhibition In Vivo

To determine the anti-tumor activity of intravenously (IV) administered CLDN-18.2-targeting RNA composition in a CLDN-18.2+ human gastric carcinoma xenograft tumor model, Hsd:Athymic Nude-Foxn1 nu/nu mice were subcutaneously inoculated with 5×10⁶ CLDN-18.2+ NCI-N87 transfectants. Mice with established tumors (mean ≥30 mm 3) received six single IV bolus injections of 3 μg, 10 μg and 30 μg CLDN-18.2-targeting RNA composition, 30 μg control mRNA encoding luciferase, saline or 800 μg of the reference protein IMAB362 on test days 15, 22, 29, 36, 43 and 50. Significant tumor growth inhibition compared to the controls was observed after the 3^{rd} dosing cycle with 30 μg CLDN-18.2-targeting RNA composition. The anti-tumor activity of 30 μg CLDN-18.2-targeting RNA composition was comparable to the tumor growth retardation gained with 800 μg of the reference protein IMAB362 (FIG. 6).

### Example 5: Safety Pharmacology Assessment of CLDN-18.2-Targeting RNA Compositions

GLP compliant assessment of CNS and respiratory safety was conducted in mice after repetitive dosing. Potential effects of CLDN-18.2-targeting RNA compositions on the blood pressure of non-human primates (NHPs) after repetitive dosing were assessed in a non-GLP PK/tolerability study. All studies were designed in an ICH S7A-compliant manner (Table 4).

TABLE 4
Overview of exemplary safety pharmacology studies.

| | | Dose levels | |
|---|---|---|---|
| Type of study | Animal species | tested (mg/kg) | Parameters measured |
| Respiratory | Balb/c mice/ | 1: Saline | Respiratory rate |
| safety | | 2: Empty lipid | Tidal volume |
| | | nanoparticles | Minute volume |
| | | (LNP) | Peak inspiratory flow (PIF) |
| | | 3: 1.5 | Peak expiratory flow (PEF) |
| | | 4: 5 | Inspiratory time (Ti) |
| | | | Expiratory time (Te) |
| Neurological | Balb/c mice/ | 1: Saline | Observational screening |
| safety | | 2: Empty LNP | (awareness, mood, motor activity, |
| | | 3: 1.5 | CNS excitation, posture, muscle |
| | | 4: 5 | tone, reflexes, autonomy) |
| | | | Hind leg splay |
| | | | Functional tests (grip strength, |
| | | | locomotor activity) |
| Cardiovascular | Cynomolgus | 1: Saline | Systolic blood pressure |
| safety | monkeys | 2: Empty LNP | Diastolic blood pressure |
| | | 3: 0.1 | |
| | | 4: 0.4 | |
| | | 5: 1.6 | |

Central nervous and respiratory system safety. GLP-compliant sub-chronic toxicity study was conducted to assess the effect of repeat intravenous bolus injection of CLDN-18.2-targeting RNA composition in male and female mice. The study included safety pharmacology assessments of satellite animals, as indicated below (Table 5).

TABLE 5
Neurological and respiratory safety assessment within
the GLP sub-chronic repeated dose toxicity study

| Test Item | Exemplary CLDN-18.2-targeting RNA composition | |
|---|---|---|
| Test system | Balb/c mice | |
| Administration | 4 administrations on day 1, 8, 15 and 22 followed by a | |
| | 2-week recovery period | |
| Group size | 4/sex/group 1-4 (SA2 satellite animals) | |
| Route | intravenous slow bolus into the tail vein | |
| Dose groups | 1. Control (Saline) | |
| Dose level^{a}/ | 2. Control (Empty LNP) | |
| animal | 3. Exemplary CLDN-18.2-targeting RNA composition | |
| | 30 μg (equivalent to approx. 1.5 mg/kg) | |
| | 4. Exemplary CLDN-18.2-targeting RNA composition | |
| | 100 μg(equivalent to approx. 5 mg/kg) | |
| | Test | Times of whole body plethysmography |
| | day | in relation to dosing |
| Time points | 8 | Predose |
| [Respiratory | | 4-8 h after 2nd dosing |
| Safety] | 9 | 24-28 h after 2nd dosing |
| | 22 | Predose |
| | | 4-8 h after 4th dosing |
| | 23 | 24-28 h after 4th dosing |
| | Test | Times of neurological screening |
| | day | in relation to dosing |
| Time points | 1 | Prior to 1st dosing |
| [Neurological | 3 | 48 h after 1st dosing |
| Safety] | 22 | Prior to 4th dosing |
| | 24 | 48 h after 4th dosing |

^{a}Dose levels expressed as the total mRNA dose

To assess the respiratory safety of a CLDN-18.2-targeting RNA composition, plethysmography was conducted pre-dose, four hours and 24 hours post-dose for the second and fourth injection. Respiratory rate, tidal volume, minute volume, peak inspiratory flow, peak expiratory flow, inspiratory time, expiratory time and airway resistance index were assessed every 10 minutes from 10 to 60 minutes (pre- and post-dose) and every 30 minutes from 1 to 4 hours (post-dose) for the measurement after test item administration to give a mean value for each time period.

Animals underwent neurological testing pre-dose and 48 hours after the first and fourth injection. Awareness, mood, motor activity, CNS excitation, posture, muscle tone, reflexes and autonomic body temperature, hind leg splay, grip strength and locomotor activity were tested.

Statistically-significant changes (p≤0.05) were seen for one parameter: male mice receiving 100 μg CLDN-18.2-targeting RNA composition/animal showed a decrease in grip strength 48 hours after the first dose (p≤0.01); female mice receiving 100 μg CLDN-18.2-targeting RNA composition/animal showed a similar decrease in griping strength after the first injection (p≤0.05).

Gastric safety. Without wishing to be bound by theory, CLDN-18.2 target is expressed in healthy tissues of stomach in human and murine (Tureci et al. 2011). Macroscopical and histopathological assessment of the stomach was included in the GLP-compliant repeated-dose toxicity study in mouse (See Example 7).

Cardiovascular safety. In a PK/Tolerability study blood pressure measurements were performed before first dosing and 24 h after the third dosing of the animals (study designed is described in Example 7).

The peripheral arterial systolic and diastolic blood pressure as well as the resulting mean blood pressure were within the normal physiological limits in the test item-treated animals.

### Example 6: Pharmacokinetics Assessment of CLDN-18.2-Targeting RNA Compositions

The pharmacokinetics of the lipid nanoparticles (LNP) formulated RNAs can be split into two phases: after intravenous injection, the LNPs are distributed systemically in the circulation and deliver the RNA to the intended target organ, the liver. Secondly, liver cells are transfected by the LNP formulation, translate the RNA and secrete the encoded proteins.

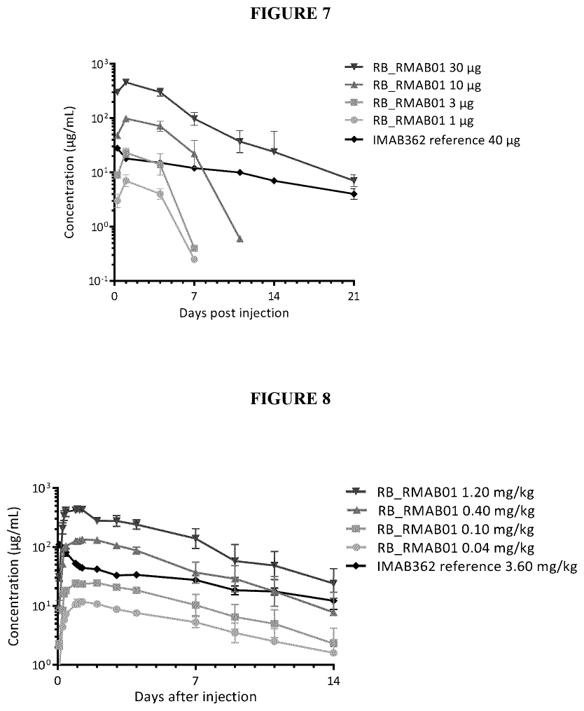

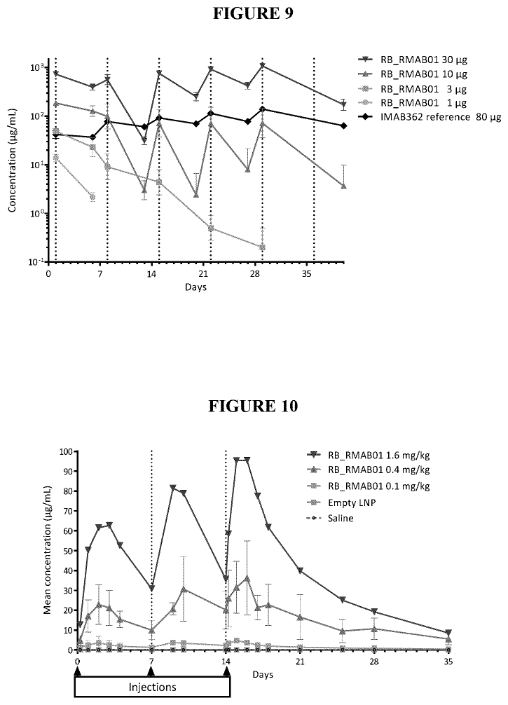

The pharmacokinetic profile of CLDN-18.2-targeting RiboMab was characterized in three different species after single dose administration [in mice (FIG. 7) and in rats (FIG. 8) and repeated dose administration [in mice (FIG. 9) and in non-human primates (FIG. 10)].

TABLE 6
Summary of exemplary studies on pharmacokinetics.

| Species | Type | Result |
|---|---|---|
| Mouse | Kinetics in blood | CLDN-18.2-targeting RiboMab is expressed in a dose- |
| | (single dose) | dependent manner with a Cₘₐₓ at approx. 450 μg/mL |
| | | with the highest dose after 24 hours post administration. |
| | | A CLDN-18.2-targeting antibody agent was detectable |
| | | up to 504 hours post dosing. |
| Rat | Kinetics in blood | Peak concentrations, similar to mice, of approx. 450 |
| | (single dose) | μg/mL were reached with the highest dose and CLDN- |
| | | 18.2-targeting RiboMab was detectable until the |
| | | termination of the study 336 hours post administration |
| | | in all dose groups |
| Mouse | Kinetics in blood | Sustained CLDN-18.2-targeting RiboMab |
| | (repeated dose) | concentrations achieved by weekly administration of |
| | | CLDN-18.2-targeting RNA composition in mice. |
| Non- | Kinetics in blood | CLDN-18.2-targeting RNA composition is sustainably |
| Human | (repeated dose) | expressed in NHP following weekly administration |
| Primates | | (detection for up to 35 days after last dosing with a Cₘₐₓ |
| (NHP) | | after 48 to 72 hours). |
| Mouse | LNP organ | LNP encapsulated mRNA is targeted to and mainly |
| | targeting | expressed in the liver with a duration of up to 72 hours. |

To assess the PK of CLDN-18.2-targeting RiboMab translated from CLDN-18.2-targeting RNA composition, a single dose PK study in Balb/c JRj mice was performed. Treatment groups received an IV bolus injection of 1 μg (0.040 mg/kg), 3 μg (˜0.10 mg/kg), 10 μg (˜0.40 mg/kg) or 30 μg (˜1.20 mg/kg) CLDN-18.2-targeting RNA composition and 40 μg (˜1.60 mg/kg) IMAB362 reference protein as internal control. Plasma was sampled 6, 24, 96, 168, 264, 336 and 504 hours post administration and CLDN-18.2-targeting RiboMab concentrations were assessed via ELISA. CLDN-18.2-targeting RiboMab concentrations displayed a CLDN-18.2-targeting RNA composition concentration-dependent expression with a peak at 24 hours post administration and a gradual decrease thereafter. Peak concentrations of approximately 450 μg/mL were reached with the highest dose and CLDN-18.2-targeting RiboMab concentrations were detectable up to 504 hours post administration (FIG. 7). The results show that CLDN-18.2-targeting RiboMab is expressed in a dose-dependent manner in mice after single dosing.

To assess the PK of CLDN-18.2-targeting RiboMab translated from CLDN-18.2-targeting RNA composition in a larger rodent organism a single dose study was performed in RjHan:Wister rats. Treatment groups received an IV bolus dose of either 0.04 mg/kg, mg/kg, 0.40 mg/kg or 1.20 mg/kg CLDN-18.2-targeting RNA composition and 3.60 mg/kg IMAB362 reference protein. Plasma was sampled 2, 6, 8, 10, 22, 24, 27, 30, 48, 72, 96, 168, 216, 264 and 336 hours post administration and CLDN-18.2-targeting RiboMab concentrations were determined via ELISA. CLDN-18.2-targeting RiboMab revealed a CLDN-18.2-targeting RNA composition concentration-dependent expression with a peak at 24 hours post administration and a gradual decrease thereafter. Peak concentrations, similar to mice (FIG. 7), of approximately 450 μg/mL were reached with the highest dose and CLDN-18.2-targeting RiboMab concentrations were detectable until the termination of the study 336 hours post administration in all dose groups (FIG. 8). The results show that CLDN-18.2-targeting RiboMab expression level in rats can be similar to mice.

A repetitive dose PK study was performed in Balb/cJRj mice to assess whether CLDN-18.2-targeting RiboMab concentrations were maintained by weekly administration of CLDN-18.2-targeting RNA composition. Treatment groups received five IV bolus doses of 1 μg (˜0.04 mg/kg), 3 μg (˜0.10 mg/kg), 10 μg (˜0.40 mg/kg) or 30 μg (˜1.20 mg/kg) CLDN-18.2-targeting RNA composition and 80 μg (˜3.20 mg/kg) IMAB362 reference protein as internal control at a weekly interval. Plasma was sampled 24 hours pre- and 24 hours post-dosing (Cₘₐₓ) respectively and concentrations of CLDN-18.2-targeting RiboMab were determined via ELISA. Repeated administration of CLDN-18.2-targeting RNA composition resulted in sustained CLDN-18.2-targeting RiboMab levels with a peak concentration of up to ˜1000 μg/mL (30 μg CLDN-18.2-targeting RNA composition) without loss in translation (FIG. 9). The results show that sustained CLDN-18.2-targeting RiboMab concentrations can be reached by weekly administration of CLDN-18.2-targeting RNA composition in mice.

A repetitive dose PK study of CLDN-18.2-targeting RNA composition was conducted in NHP as a phylogenetic and physiological closely related organism to humans (see Table 7 for description of an exemplary study design).

TABLE 7
Exemplary study design of the PK/Tolerability study in NHPs

| Test Item | CLDN-18.2-targeting RNA composition |
|---|---|
| Test system | M. fascicularis |
| Administration | 3 Administrations on day 1, 8 and 15 followed by a 3- |
| | week recovery period |
| Group size | 3 Females/group |
| Route | Intravenous bolus |
| Dose groups - | Control (Saline) - 1.6 mL/kg |
| Dose level^{a}/ | Control (Empty LNP) - 1.6 mL/kg |
| animal | RB_RMAB01 0.1 mg/kg-0.1 mL/kg |
| | RB_RMAB01 0.4 mg/kg-0.4 mL/kg |
| | RB_RMAB01 1.6 mg/kg-1.6 mL/kg |
| Safety readouts | Mortality, clinical observations, cage side observations, |
| | body weight, food consumption, and local tolerance |
| | Ophthalmology (pre-dose, +24 h post third dosing and |
| | at the end of the 3-week recovery period) |
| | Hematology, coagulation and clinical chemistry (pre- |
| | dose, +24 h post third dosing and at the end of the 3- |
| | week recovery period) |
| | Cytokines IFNα, IFNγ, IL-1β, IL-2, IL-6, IL-8 IL-10, |
| | IL-12p70 and TNFα (pre-dose, +6 h, +24 h and +48 |
| | h after first and third dosing) |
| | Urinalysis (pre-dose, +48 h post last dosing and at the |
| | end if the 3-week recovery period) |
| PK sampling | +6 h, +24 h, +48 h, +72 h, +96 h and +7 d after first dosing (prior to second |
| | dosing) |
| | +48 h, +72 h and +7 d after second dosing (prior to third |
| | dosing) |
| | +6 h, +24 h, +48 h, +72 h, +96 h, +7 d, +11 d, +14 d and |
| | +21 d after third dosing |
| Time points | Prior to first dose, +24 h post third dose and at the end |
| | of the 3-week |
| [Cardiovascular | observation period |
| safety] | |

^{a}Dose levels expressed as the total mRNA dose

Treatment groups received three IV bolus injections of either 0.1 mg/kg, 0.4 mg/kg or 1.6 mg/kg at weekly intervals. As controls, saline or empty LNPs were administered likewise. Serum was sampled 6, 24, 48, 72, 96 and 168 hours post 1^{st} and 3^{rd} dosing and 48, 72 and 168 hours post 2^{nd} dosing as well as 264, 336 and 504 hours post 3^{rd} dosing. Concentrations of CLDN-18.2-targeting RiboMab were analyzed via ELISA. CLDN-18.2-targeting RiboMab displayed a CLDN-18.2-targeting RNA composition dose-dependent expression with a peak between 48-72 hours post administration and a gradual decrease thereafter. Peak serum concentrations of 231.7 μg/mL were reached with the highest dose 48-72 hours post 3^{rd} administration of CLDN-18.2-targeting RNA composition and CLDN-18.2-targeting RiboMab was detectable until the termination of the study 840 hours post 1^{st} dosing (FIG. 10). The results show that weekly administration of CLDN-18.2-targeting RNA composition can result in sustainable CLDN-18.2-targeting RiboMab expression in NHP.

Distribution: Biodistribution of CLDN-18.2-targeting RNA composition was studied in mice after a single IV injection. Messenger RNA and lipid nanoparticles (LNPs) in murine tissues were quantified via digital droplet PCR (mRNA) or liquid scintillation spectrometry (radiolabeled LNP), respectively. Organ targeting and expression of LNPs encapsulating luciferase-encoding mRNA were studied via bioluminescence imaging.

mRNA distribution: A single dose of 100 μg CLDN-18.2-targeting RNA composition/animal was administered to Balb/c mice (3/sex/time point) IV and blood and tissues (spleen, lungs, liver, kidneys, heart and brain) were sampled 0.083 (5 minutes), 0.5, 6, 24, 72 and 168 hours post administration.

TABLE 8
Exemplary design of mRNA biodistribution study

| | | | # of | | |
|---|---|---|---|---|---|
| Group | | Dose^{a} | Animals | | Time Points |
| Number | Test Item | (mg/kg) | M | F | [h] |
| 1 | PBS | 0 | 4 | 4 | N/A |
| 2 | RB_RMAB01 | 5 | 21 | 21 | 0.083 [5 min], 0.5, |
| | | | | | 6, 24, 72, 168 |

| | |
|---|---|
| Tissues | Blood, spleen, liver, kidneys, heart, thymus, lungs, brain |

^{a}Dose levels expressed as the total mRNA dose

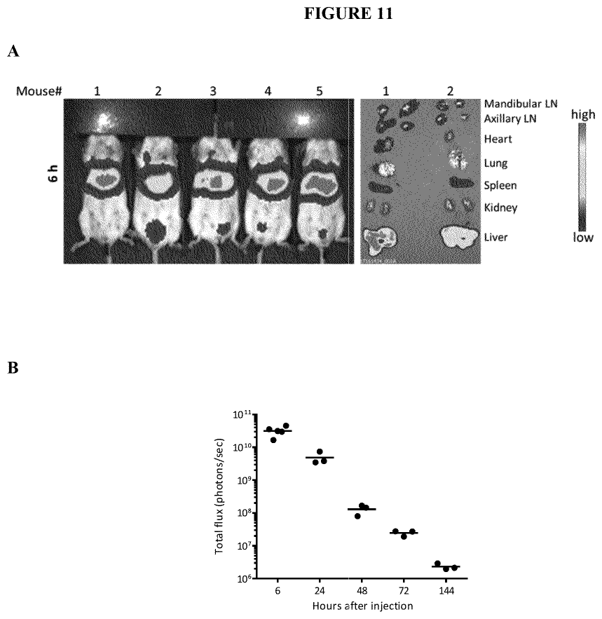

LNP distribution: Biodistribution of lipid nanoparticles (LNP) was assessed with modified mRNA encoding firefly luciferase formulated with lipid nanoparticles (LNPs) to assess liver targeting and kinetics of in vivo translated mRNA. Following IV administration, the luciferase protein showed a time-dependent translation with high bioluminescence signals mainly located in the liver (FIG. 11). The results show that LNP encapsulated mRNA can be targeted to and expressed in the liver.

The tissue distribution profile of LNP of CLDN-18.2-targeting RNA composition was investigated in CD-1 mice (4/sex/time point) after a single IV bolus injection at 1 mg/kg. [³H]-CLDN-18.2-targeting RNA composition was used for this analysis and the particles contained a non-exchangeable, non-metabolizable LNP marker, [³H]-cholesteryl hexadecyl ether ([³H]-CHE). An exemplary study design is depicted in Table 9 below.

TABLE 9
Exemplary study design of LNP biodistribution study

| | | | # of | | |
|---|---|---|---|---|---|
| Group | | Dose^{a} | Animals | | Time Points |
| Number | Test Item | (mg/kg) | M | F | [h] |
| 1 | PBS | 0 | 4 | 4 | N/A |
| 2 | [³H]-CLDN- | 1 | 32 | 32 | 0.083, 0.25 ^{b}, 0.5, |
| | 18.2-targeting | | | | 1 ^{b}, 2, 4 ^{b}, 8, 24^{b} |
| | RNA | | | | |
| | composition | | | | |

| | |
|---|---|
| Tissues | Blood, plasma, spleen, pancreas, liver, kidneys, adrenal glands, |
| | ovaries/testes, small intestine, large intestine, lymph nodes |
| | [brachial/inguinal/mesenteric], heart, thymus, lungs, muscle, |
| | brain [no brainstem], bone marrow |

^{a}Dose levels expressed as the total mRNA dose
^{b} At bold time points tissues were collected in addition to blood and plasma

Mice were euthanized, blood and plasma collected at 0.083 (5 min), 0.25, 0.5, 1, 2, 4, 8 and 24 hours post-dose. Tissues were only sampled at 0.25, 1, 4 and 24 hours post-dose. Radioactivity in all samples was determined by standard liquid scintillation counting (LSC) and the resulting values used to calculate total and relative lipid concentrations.

[³H]-CLDN-18.2-targeting RNA composition exhibited bi-phasic kinetics in blood and plasma in mice, with a rapid initial decline in blood/plasma concentrations, followed by a slower elimination phase. The distribution of [³H]-CLDN-18.2-targeting RNA composition into tissues was rapid, with peak levels observed in all tissues by 0.5-2 hours post-dose. The principal tissues/organs of distribution for [³H]-CLDN-18.2-targeting RNA composition were the liver and the spleen (˜70-74% and ˜0.8-1.2% of the injected dose present in the liver and the spleen, respectively, at 4 hours after injection) and minimal distribution was observed into other tissues. A summary of the calculated total lipid concentrations (i.e., of all 4 administered lipids) and calculated % of injected dose of [³H]-CLDN-18.2-targeting RNA composition in various tissues is shown in Table 10.

TABLE 10
Tissue levels of total lipids (from CLDN-18.2-targeting RNA
composition) at 4 hours after IV bolus injection in CD-1 mice

| CLDN-18.2-targeting RNA composition | |
|---|---|
| μg Lipid/g tissue^{a} | % Injected Dose |

| Tissue | Females | Males | Females | Males |
|---|---|---|---|---|
| Liver | 355.19 | 282.43 | 70.12 | 74.12 |
| Spleen | 77.08 | 61.67 | 1.15 | 0.84 |
| Plasma | 28.54 | 26.24 | 4.18 | 4.06 |
| Adrenal glands | 17.64 | 6.65 | 0.03 | 0.02 |
| Lymph nodes | 7.66 | 5.24 | 0.06 | 0.04 |
| Thymus | 4.80 | 3.86 | 0.07 | 0.04 |
| Small intestine | 4.23 | 3.72 | 0.65 | 0.53 |
| Lung | 3.81 | 3.58 | 0.10 | 0.10 |
| Heart | 2.90 | 2.71 | 0.05 | 0.05 |
| Large intestine | 1.68 | 2.36 | 0.06 | 0.07 |
| Pancreas | 3.51 | 1.91 | 0.07 | 0.04 |
| Ovaries/testes | 5.88 | 1.53 | 0.03 | 0.04 |
| Kidney | 2.40 | 1.52 | 0.12 | 0.10 |
| Muscle | 0.55 | 0.69 | 0.03 | 0.05 |
| Brain | 0.04 | 0.03 | 0.00 | 0.00 |
| Bone marrow | NC | NC | 0.09^{d} | 0.06^{d} |

^{a}Tissues listed according to the rank-ordered mean concentrations (highest to lowest) in male mice at 4 hours post-injection
^{b}NC, Not calculated; weight of entire tissue was not available
^{c}BLQ, Below limit of quantification
^{d}Based on both femurs

Metabolism and excretion: Messenger RNA, including pseudouridine modified mRNA is generally sensitive to degradation by cellular RNases and subjected to nucleic acid metabolism. Nucleotide metabolism occurs continuously within the cell with the nucleoside being degraded to waste products and excreted or recycled for nucleotide synthesis.

In some embodiments of a CLDN-18.2-targeting RNA composition described herein, such a composition comprises a plurality of lipids, some of which can be naturally occurring (e.g., in some embodiments neutral lipids such as, e.g., cholesterol and DSPC). A skilled artisan reading the present disclosure may expect that metabolism and excretion of naturally occurring lipids can be similar to that of endogenous lipids. A skilled artisan reading the present disclosure will also understand that the metabolism and excretion of other lipids within a CLDN-18.2-targeting RNA composition (e.g., a conjugated lipid and a cationic lipid) can be characterized using methods known in the art.

In some embodiments, the structure of an expressed CLDN-18.2-targeting RiboMab is based on an IgG1 antibody. In some such embodiments, its metabolism can be similar to that of endogenous IgG1 molecules. Exemplary metabolism includes, but is not limited to, degradation to small peptides and amino acids.

### Example 7: Toxicology Assessment of CLDN-18.2-Targeting RNA Compositions

The toxicology assessment of CLDN-18.2-targeting RNA compositions can comprise in vitro studies using human blood components and in vivo studies in mouse and cynomolgus monkey. Drug product haematocompatibility with human blood can be assessed in vitro, while toxicities mediated by the CLDN-18.2-targeting RNA compositions (RNA and LNP) as well as by the translated CLDN-18.2-targeting RiboMab (protein) can be detected in the selected in vivo models. A summary of certain features assessed in non-clinical studies is given in Table 11 below.

TABLE 11
Exemplary non-clinical safety and toxicology studies of CLDN-
18.2-targeting RNA compositions and the encoded antibody.

| Topic | Species | Type | Result/Observations | Reference |
|---|---|---|---|---|
| Immunotoxicity | Human | Non-GLP | No activation of human | Example 7 |
| | (serum) - | complement | complement in normalized | |
| | in vitro | activation | serum through | |
| | | | quantification of C3a, C4a, | |
| | | | C5a and the terminal | |
| | | | complement complex SC5b- | |
| | | | 9 was observed | |
| Immunotoxicity | Human | Non-GLP | No cytokine release was | Example 7 |
| | (whole | Cytokine | measurable upon drug | |
| | blood) - | Release | product incubation in vitro | |
| | in vitro | | with human whole blood | |
| Toxicology/ | Cynomolgus- | Non-GLP | No test-item mediated | Example 7 |
| PK | in vivo | PK/Tolerability | changes in blood | |
| | | Study | parameters (hematology, | |
| | | | clinical chemistry), cytokine | |
| | | | release were observed upon | |
| | | | repeated drug product | |
| | | | administration | |
| Safety | Mouse - | GLP Single- | Assessing 40 | Example 5 |
| Pharmacology | in vivo | Dose CNS | neuropharmacological | |
| | | Safety Study | parameters after drug | |
| | | | product application | |
| Safety | Mouse - | GLP Single- | Determining 8 respiratory | Example 5 |
| Pharmacology | in vivo | Dose | parameters after drug | |
| | | Respiratory | product application using | |
| | | Safety Study | whole body | |
| | | | plethysmography | |
| Safety | Cynomolgus- | Non-GLP | No effect of the drug | Example 5 |
| Pharmacology | in vivo | PK/Tolerability | product on the blood | |
| | | Study | pressure was observed after | |
| | | | repeated administration | |
| Toxicology | Mouse - | Non-GLP | Assessing toxicity after | Example 7 |
| | in vivo | Single Dose | single drug product | |
| | | Toxicity Study | application (final report | |
| | | | pending) | |
| Toxicology | Mouse - | GLP 3-week | Assessing toxicity of weekly | Example 7 |
| | in vivo | Repeated-Dose | drug product application | |
| | | Toxicity Study | | |

In some embodiments, relevant species for assessment of the antibody (CLDN-18.2-targeting RiboMab) mediated toxicity are mouse and cynomolgus monkey, due to the highly conserved protein sequence and equal expression pattern of the CLDN-18.2 target in these species (Türeci et al. 2011).

Single-dose toxicology. A single-dose toxicity study was conducted in male and female CD-1 mice to: i) characterize the potential toxicity of CLDN-18.2-targeting RNA compositions, ii) compare the toxicity of CLDN-18.2-targeting RNA compositions with the respective control item (e.g., empty lipid nanoparticles), and iii) assess the reversibility, progression and/or potential delayed effects of CLDN-18.2-targeting RNA compositions after a 4-week observation period (termination on Day 29).

Mice received a single IV dose of a CLDN-18.2-targeting RNA composition (at a total mRNA dose level of 1, 2, or 4 mg/kg) or control item (e.g., empty nanoparticles or saline control) on Day 1 by IV administration. Animals were euthanized on Day 3 (main animals) and Day 29 (recovery/delayed findings). Study endpoints included mortality, clinical observations, body weight changes, clinical chemistry, necropsy observations, organ weights, and histopathology (liver, spleen and stomach).

A single IV dose of CLDN-18.2-targeting RNA composition at 1, 2 and 4 mg/kg, or Empty LNP, was generally well-tolerated in male and female CD-1 mice. There was no mortality during the 28-day observation period. Minor findings were noted on Day 3 regarding liver parameter and spleen weight increase. Minor findings in microscopic assessment of liver and spleen were considered non-adverse. All findings were resolved after the recovery period at Day 29.

Repeated-dose toxicology. A 21-day GLP-compliant repeated-dose toxicity study was conducted in Balb/c mice with weekly intravenous bolus administrations of CLDN-18.2-targeting RNA composition followed by a 2-week recovery period (see Table 12 for an exemplary study design). Study readouts include, but are not limited to, clinical signs of intolerance (e.g., ptosis, piloerection, reduced motility and/or cold to touch), mortality, body weight and food consumption, local tolerance, hematology, clinical chemistry (e.g., globulin, albumin, cholesterol, creatinine, total protein, blood glucose, alkaline phosphatase (aP), lactate dehydrogenase (LDH), and aspartate aminotransferase (ALAP), and glutamate dehydrogenase (GLDH) blood levels), urine analysis, ophthalmology and auditory system, macroscopic post-mortem findings, organ weights, bone marrow, histopathology, and cytokines (e.g., IL-6, TNF-α, IFN-α, IFN-γ, IL-1β, IL-2, IL-10, and/or IL-12p70).

TABLE 12
Exemplary design of the GLP-compliant
repeated-dose toxicity study

| Test Item | CLDN-18.2-targeting RNA composition |
|---|---|
| Test system | Balb/c mice |
| Administration | 4 administrations on day 1, 8, 15 and 22 followed by |
| | a 2-week recovery period |
| Route | intravenous slow bolus into the tail vein |
| Dose groups | 1. Control (Saline) |
| Dose level/animal | 2. Control (Empty LNP) |
| | 3. RB_RMAB01 30 μg (equivalent to approx. 1.5 |
| | mg/kg) |
| | 4. RB_RMAB01 100 μg (equivalent to approx. 5 |
| | mg/kg) |
| Satellite groups | SA1: for cytokine and dose exposure sampling |
| | SA2: for safety pharmacology assessment |
| | (respiratory and neurological safety) |
| Group size | Group 1-4: 10 + 5 recovery/sex/group |
| | SA1: 9/sex/group 1-4 |
| | SA2: 4/sex/group 1-4 |

Immunotoxicity: Haematocompatibility of CLDN-18.2-targeting RNA compositions was determined in vitro in human serum and blood, testing for drug product mediated complement activation and cytokine release, respectively. Furthermore, immunotoxicity in vivo was assessed as part of the repeated-dose toxicity study in mice and a pharmacokinetic study in cynomolgus monkey. All studies were designed in accordance with the ICH S8 guideline (Immunotoxicity studies for human pharmaceuticals).

Preliminary results of the toxicity study show that IL-6 and TNF-α were transiently elevated 6 h post administration in the empty LNP control group and in both dose groups (30 and 100 μg CLDN-18.2-targeting RNA composition/animal), while IFN-α and IFN-γ were transiently elevated in both dose groups. Plasma levels returned to baseline by 48 h post administration. No elevation of IL-1β, IL-2, IL-10 or IL-12p70 was observed in any of the groups.

In the PK/Tolerability study in cynomolgus monkey as described in Example 6, no cytokine elevation was observed in any of the groups.

In vitro complement activation of human serum. The potential of CLDN-18.2-targeting RNA composition to activate human complement in vitro was evaluated through incubation in normal human serum with drug product concentrations selected based on plasma Cₘₐₓ levels at doses associated with toxicity for similar lipid nanoparticle products (e.g., containing siRNA) administered to humans (Fitzgerald et al. 2014; Coelho et al. 2013; Tabernero et al. 2013; Patisaran FDA approval 2017). Complement activation was assessed by evaluating levels of complement split products, C3a, C4a, C5a, using a multiplex cytometric bead array, and the terminal complement complex, SC5b-9, using an enzyme immunoassay.

The in vitro incubation of CLDN-18.2-targeting RNA composition with normal human serum complement resulted in no increases in complement split products or terminal complement complex when compared with negative controls, while expected activation was induced by the positive control. In summary, CLDN-18.2-targeting RNA composition did not activate human complement in vitro under the conditions tested.

Whole blood cytokine release. In some embodiments, a CLDN-18.2-targeting RNA composition described herein may be administered parenterally. In such embodiments, a CLDN-18.2-targeting RNA composition may be in contact with peripheral blood mononuclear cells (PBMCs) during circulation in the blood. One of ordinary skill in the art reading the present disclosure will appreciate that interaction between the drug product and blood components may lead to an induction of cytokine secretion. Therefore, in vitro tolerability of an exemplary CLDN-18.2-targeting RNA composition was investigated using human whole blood. For example, secretion of pro-inflammatory cytokines (e.g., but not limited to IFN-α, IFN-γ, IL-1β, IL 2, IL-6, IL-8, IL-12p70, IP-10, and/or TNF-α) was evaluated after incubation of a dilution range representative of anticipated concentrations in human blood. No test item-related induction of cytokine secretion was detectable in this assay and the in vitro tolerability could be shown.

### Example 8: Exemplary Dosing (e.g. Dose Escalation)

In some embodiments, pharmaceutical compositions provided herein can be administered to patients with CLDN-18.2 positive cancer as monotherapy and/or in combination with other anti-cancer therapies.

In some embodiments, administration involves one or more cycles. In some embodiments, pharmaceutical compositions provided herein can be administered in at least 3-8 cycles.

In some embodiments, a dosing regimen, and in particular a monotherapy dosing regimen, may be or comprise dosing every 21 days (Q3W).

In some embodiments, dose escalation may be performed. In some such embodiments, dosing may be performed at one or more of the levels shown in Table 13 below; in some embodiments, dose escalation may involve administration of at least one lower dose from Table 13 followed later by administration of at least one higher dose from Table 13.

TABLE 13
Exemplary Dosing

| | | |
|---|---|---|
| Dose Level | Dose¹ | Dose Increment² |

| | | | |
|---|---|---|---|
| 1 | 0.15 | mg/kg | Exemplary starting dose |
| 2 | 0.30 | mg/kg | 100% |
| 3 | 0.60 | mg/kg | 100% |
| 4 | 1.0 | mg/kg | 66.7% |
| 5 | 1.5 | mg/kg | 50% |
| 6 | 2.0 | mg/kg | 33.3% |
| 7 | 2.5 | mg/kg | 25% |
| 8 | 3.0 | mg/kg | 20% |

¹As presented in Table 13, a “dose” refers to total RNA dose.
²Dose Increment presented in Table 13 relative to the dose immediately above, beginning with the indicated exemplary starting dose

In some embodiments, additional or alternative doses levels may be evaluated, for example, including, e.g., dose levels at 0.2, 0.225, 0.25, 0.35, 0.4, 0.45, 0.5, 0.55, 0.65, 0.7, 0.75, 0.80, 0.85, 0.9, 0.95, 1.25, 1.75, 2.25, 2.75, 3.25, 3.5, and 4 mg/kg.

Efficacy of a treatment can be assessed by on-treatment imaging, for example, at Week 6 (+7 days), every 6 weeks (±7 days) for 24 weeks, and every 12 weeks (±7 days) thereafter.

### Example 9: Approved Therapies for Treatment of Certain Cancers

Approved therapies are available for certain cancers associated with CLDN-18.2 expression. For example, erlotinib, an epidermal growth factor receptor (EGFR) inhibitor is the only targeted therapy approved in the US in combination with gemcitabine for the first-line treatment of patients with locally advanced, unresectable or metastatic pancreatic cancer. However, a randomized controlled trial (RCT) comparing erlotinib versus placebo showed a 0.4-month median overall survival (OS) benefit and 0.3-month median progression-free survival (PFS) benefit.

In some embodiments, the recommended daily dose of erlotinib (e.g., erlotinib hydrochloride) for treatment of pancreatic cancer is about 109 mg taken at least one hour before or two hours after the ingestion of food, in combination with gemcitabine. In some embodiments, the recommended dose of gemcitabine (Gemzar) for treatment of pancreatic cancer is 1000 mg/m² over 30 minutes once weekly for the first 7 weeks, then one week rest, the one once weekly for 3 weeks of each 28-day cycle.

### Example 10: Exemplary Adverse Events

In some embodiments, subjects to whom a pharmaceutical composition as described herein is administered may be monitored over a period of treatment regimen for one or more indicators of a potential adverse event. For example, in some embodiments, subjects may be monitored for one or more hematologic toxicities (e.g., presence of neutropenia, thrombocytopenia, and/or anemia, etc.) and/or non-hematologic toxicities (e.g., elevation of alanine aminotransferase (ALT), aspartate aminotransferase (AST), and/or bilirubin, etc.).

### Example 11: Exemplary Assessments and/or Criteria for Single-Stranded RNAs Described Herein

In some embodiments, one or more assessments as described herein may be utilized during manufacture, or other preparation or use of single stranded RNAs (e.g., as a release test).

In some embodiments, one or more quality control parameters may be assessed to determine whether single-stranded RNAs described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Methods for assessing RNA quality are known in the art; for example, one of skill in the art will recognize that in some embodiments, one or more analytical tests as described in Table 14 can be used for RNA quality assessment.

In some embodiments, a batch of single stranded RNAs may be assessed for the following features listed in Table 14 to determine next action step(s). For example, a batch of single stranded RNAs can be designated for one or more further steps of manufacturing and/or formulation and/or distribution if RNA quality assessment indicates that such a batch of single stranded RNAs meet or exceed the acceptance criteria listed in Table 14. Otherwise, an alternative action can be taken (e.g., discarding the batch) if such a batch of single stranded RNAs does not meet or exceed the acceptance criteria.

In some embodiments, a batch of single stranded RNAs with exemplary assessment results as shown in Table 14 can be utilized for one or more further steps of manufacturing and/or formulation and/or distribution.

TABLE 14
Exemplary tests and specifications for individual RNAs.

| | | | Exemplary batch results |
|---|---|---|---|
| Parameter | Example Analytical Test | Acceptance Criteria | from single stranded RNAs |
| RNA integrity | Capillary gel | ≥90.0% in the peak | 100% |
| | electrophoresis | corresponding to intact RNA | |
| Content (RNA | UV absorption | Desirable concentrations | 1.72 mg/mL |
| concentration) | spectrophotometry (Ph. | (e.g., 1-2 mg/mL) | |
| | Eur. 2.2.25); A_{260 nm} | | |
| Residual DNA | Quantitative PCR | ≤50 ng per mg RNA | 0.09-0.68 ng DNA/mg |
| template | | | RNA |
| Residual | Immuno-based assay | ≤100 pg dsRNA/μg RNA | ≤100 pg dsRNA/μg RNA |
| dsRNA | | | |
| Protein | Detection of translated | Complies | Complies |
| expression | protein | | |

### Example 12: Exemplary Assessments and/or Criteria for Compositions Containing Two or More RNAs

In some embodiments, one or more assessments as described herein may be utilized during manufacture, or other preparation or use of a drug substance (e.g., as a release test).

In some embodiments, a batch of a first single stranded RNA encoding a heavy chain of CLDN-18.2-targeting antibody and a batch of a second single stranded RNA encoding a light chain of CLDN-18.2-targeting antibody are assessed for one or more features as described in Example 11. In some such embodiments, batches of a first and a second ssRNA that both meet or exceed acceptance criteria as listed in Table 14 are then mixed together, for example, in a molar ratio of about 1.5:1 to about 1:1.5, to form an RNA drug substance. In some embodiments, such an RNA drug substance may be assessed for one or more quality control parameters (e.g., for release and/or for further manufacturing) including, e.g., but are not limited to physical appearance, RNA length, identity (as RNA), integrity, sequence, and/or concentration, pH, osmolality, RNA ratio (e.g., ratio of a HC RNA to a LC RNA), potency, bacterial endotoxins, bioburden, and combinations thereof. Such quality control parameters can be assessed by one or more of certain analytical methods known in the art, such as, e.g., visual inspection, gel electrophoresis (e.g., agarose gel electrophoresis, capillary gel electrophoresis), enzymatic degradation, sequencing, UV absorption spectrophotometry. PCR methods, bacterial endotoxin testing (e.g., limulus amebocyte lysate (LAL) testing).

### Example 13: Exemplary RNA Product Formulation

In some embodiments, an exemplary RNA product formulation is a sterile RNA-lipid nanoparticle (RNA-LNP) dispersion in aqueous buffer, for example, for intravenous administration. For example, in some embodiments, such an RNA product formulation may be filled at about 0.8 to about 1.2 mg/mL, to a 5.0 mL nominal fill volume. In some embodiments, each vial may be intended for single use. In some embodiments, an RNA product formulation (e.g., as described herein) may be stored frozen at −80 to −60° C.

In some embodiments, such an exemplary RNA product formulation may comprise two or more distinct RNAs each encoding a portion of a CLDN-18.2-targeting antibody (e.g., an RNA encoding a heavy chain of a CLDN-18.2-targeting antibody and an RNA encoding a light chain of a CLDN-18.2-targeting antibody), at least one cationic lipid, at least one conjugated lipid, at least one neutral lipid, and an aqueous buffer comprising one or more salts. In some embodiments, a polymer-conjugated lipid (e.g., a PEG-conjugated lipid such as for example in some embodiments, a PEG-conjugated lipid is or comprises 2-[(polyethylene glycol)-2000]-N,N-ditetradecylacetamide) may be present in about 1-2.5 mol % of the total lipids. In some embodiments, a cationic lipid (e.g., in some embodiments, a cationic lipid being or comprising ((3-hydroxypropyl)azanediyl)bis(nonane-9,1-diyl) bis(2-butyloctanoate)) may be present in about 35-65 mol % of the total lipids. In some embodiments, a neutral lipid (e.g., in some embodiments, a neutral lipid being or comprising 1,2-Distearoyl-sn-glycero-3-phosphocholine and/or synthetic cholesterol) may be present in about 35-65 mol % of the total lipids. In some embodiments, the composition of an exemplary RNA production formulation may be characterized as shown in Table 15.

TABLE 15
Quantitative composition of an exemplary
RNA product formulation

| | | | Quantity |
|---|---|---|---|
| | | Concentration | per Unit |
| Component | Function | (mg/mL) | (mg/Vial) |
| Composition (e.g., | Active agent | 1.0 | 5.0 |
| as described herein) | | | |
| comprising two or | | | |
| more RNAs each | | | |
| encoding a distinct | | | |
| chain of a CLDN-18.2- | | | |
| targeting antibody | | | |
| Cationic lipid A^{[1]} | Functional lipid | 13.56 | 67.80 |
| PEG-conjugated lipid | Functional lipid | 1.77 | 8.85 |
| A ^{[2]} | | | |
| DSPC ^{[3]} | Structural lipid | 3.11 | 15.55 |
| Cholesterol, synthetic | Structural lipid | 6.20 | 31.00 |
| Sucrose or equivalent | Cryoprotectant | 102.69 | 513.45 |
| NaCl | Buffer | 6.00 | 30.00 |
| KCl | Buffer | 0.15 | 0.75 |
| Na₂HPO₄ | Buffer | 1.08 | 5.40 |
| KH₂PO₄ | Buffer | 0.18 | 0.90 |
| Water for injection | Solvent/Vehicle | q.s. | to 5.0 mL |

^{[1]}Cationic lipid A = ((3-hydroxypropyl)azanediyl)bis(nonane-9,1-diyl) bis(2-butyloctanoate)
^{[2]} PEG-conjugated lipid A = 2-[(polyethylene glycol)-2000]-N,N-ditetradecylacetamide
^{[3]} DSPC = 1,2-Distearoyl-sn-glycero-3-phosphocholine
q.s. = quantum satis (as much as may suffice)

### Example 14: Exemplary Lipid Excipients in an RNA/LNP Drug Product Formulation Described Herein

Materials used in a manufacturing process of the drug product can be purchased from qualified vendors, quarantined, sampled, identified, tested and released. Tests of the excipients are conducted according to pre-determined specifications or according to Ph. Eur./USP.

In some embodiments, an RNA/LNP drug product formulation comprises four lipid excipients shown in Table 16, which provides further information on the lipid excipients. All excipients are supplied as GMP-grade material.

TABLE 16
Lipid excipients in an exemplary RNA/LNP drug product described herein

| | Molecular | |
|---|---|---|
| Lipid | Weight | Chemical Name and Structure |
| Catioinc lipid A | 724 Da | 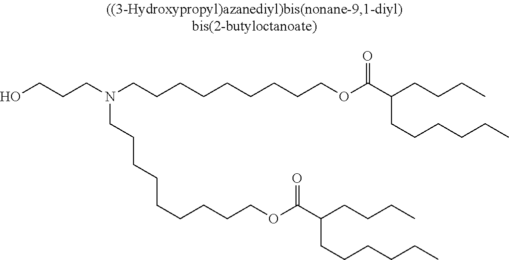 [SMILES: CCCCCCC(CCCC)C(=O)OCCCCCCCCCN(CCCO)CCCCCCCCCOC(=O)C(CCCC)CCCCCC] |
| PEG-conjugated lipid A | 2500 Da | 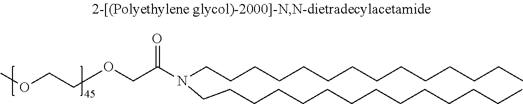 |
| Neutral lipid: DSPC | 790 Da | 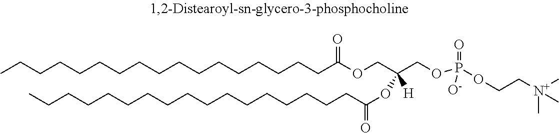 [SMILES: CCCCCCCCCCCCCCCCCC(=O)OC[C@H](COP(=O)([O-])OCC[N+](C)(C)C)OC(=O)CCCCCCCCCCCCCCCCC] |
| Neutral lipid: Cholesterol | 387 Da | 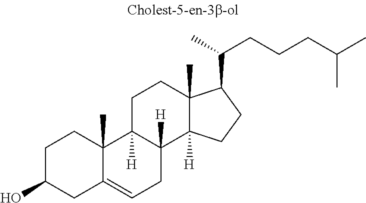 |

Cationic Lipid A: ((3-Hydroxypropyl)azanediyl)bis(nonane-9,1-diyl)bis(2-butyloctanoate)

In some embodiments, the amino lipid ((3-Hydroxypropyl)azanediyl)bis(nonane-9,1-diyl)bis(2-butyloctanoate) is a functional cationic lipid component of an RNA/LNP drug product formulation described herein. It was designed to facilitate biodegradation, metabolism and clearance in vivo. The amino lipid contains a titratable tertiary amino head group linked via ester bonds to two saturated alkyl chains which, when incorporated in LNP, confer distinct physicochemical properties that regulate particle formation, cellular uptake, fusogenicity and/or endosomal release of the RNA. The ester bonds can be hydrolyzed easily to facilitate fast degradation and excretion via renal pathways. The amino lipid has an apparent pKₐ of approximately 6.25, resulting in an essentially fully positively charged molecule at pH 5. During the manufacturing process, introduction of an aqueous RNA solution to an ethanolic lipid mixture containing the amino lipid at pH 4.0 leads to an electrostatic interaction between the negatively charged RNA backbone and the positively charged cationic lipid. This electrostatic interaction leads to particle formation coincident with efficient encapsulation of RNA drug substance. After RNA encapsulation, adjustment of the pH of the medium surrounding the resulting LNP to 7.4 results in neutralization of the surface charge of the LNP. When all other variables are held constant, charge-neutral particles display longer in vivo circulation lifetimes and better delivery to hepatocytes compared to charged particles, which are rapidly cleared by the reticuloendothelial system. Upon endosomal uptake, the low pH of the endosome renders the LNP fusogenic and allows the release of the RNA into the cytosol of the target cell.

PEG-Conjugated Lipid A: 2-[(Polyethylene glycol)-2000]-N,N-ditetradecylacetamide

In some embodiments, an RNA/LNP drug product formulation described herein contains a functional lipid excipient, 2-[(Polyethylene glycol)-2000]-N,N-ditetradecylacetamide. This PEGylated lipid is structurally similar to other clinically approved PEGylated lipids, where safety was demonstrated in clinical trials. The primary function of a PEGylated lipid is to sterically stabilize the particle by forming a protective hydrophilic layer that shields the hydrophobic lipid layer. Moreover, a PEGylated lipid reduces the association with serum proteins and the resulting uptake by the reticuloendothelial system when the particles are administered in vivo. PEG lipids are known to affect cellular uptake, a prerequisite to endosomal localization and payload delivery. It has been found that the pharmacology of encapsulated nucleic acid can be controlled in a predictable manner by modulating the alkyl chain length of the PEG-lipid anchor. In some embodiments, such PEGylated lipid was selected for an RNA/LNP drug product formulation to provide optimum delivery of RNA to the liver. In some embodiments, such selection was also based on reasonable solubility characteristics and its molecular weight to effectively perform the function of a steric barrier. Such a PEGylated lipid does not show appreciable surfactant or permeability enhancing or disturbing effects on biological membranes. Furthermore, the PEG in such a PEGylated lipid is linked to the diacyl lipid anchors with a biodegradable amide bond, facilitating fast degradation and excretion. In the vial, the particles retain a full complement of a PEGylated lipid. In the blood compartment, such a PEGylated lipid dissociates from the particle over time, revealing a more fusogenic particle that is more readily taken up by cells, ultimately leading to release of the RNA payload.

### Neutral Lipids: DSPC and Cholesterol

In some embodiments, an RNA/LNP drug product formulation comprises two or more neutral lipids. In some such embodiments, an RNA/LNP drug product formulation may comprise two or more neutral lipids, which includes DSPC and/or cholesterol. In some embodiments, such neutral lipids (e.g., DSPC and/or cholesterol) can be referred to as structural lipids with concentrations chosen to optimize LNP particle size, stability and encapsulation. For example, DSPC and cholesterol are already used in approved drug products, e.g. DSPC is used as an excipient in DaunoXome®, TOBI® Podhaler®, and Lipo-Dox®. Cholesterol is used as an excipient in Marqibo®, Doxil® and AmBisome®. Onpattro® contains both DSPC and cholesterol.

### Example 15: Exemplary Assessments and/or Criteria for RNA/LNP Drug Product Formulations Described Herein

In some embodiments, one or more assessments as described herein may be utilized during manufacture, or other preparation or use of a drug product (e.g., as a release test).

In some embodiments, a RNA/LNP drug product may be assessed for one or more quality control parameters (e.g., for release and/or for further processing) including, e.g., but are not limited to physical appearance, lipid identity and/or content, LNP size, LNP polydispersity, RNA encapsulation, RNA length, identity (as RNA), integrity, sequence, and/or concentration, pH, osmolality, RNA ratio (e.g., ratio of a HC RNA to a LC RNA), potency, bacterial endotoxins, bioburden, residual organic solvent, osmolality, pH, and combinations thereof. Such quality control parameters can be assessed by one or more of certain analytical methods known in the art, such as, e.g., visual inspection, gel electrophoresis (e.g., agarose gel electrophoresis, capillary gel electrophoresis), enzymatic degradation, sequencing, UV absorption spectrophotometry. RNA labeling dye, PCR methods, bacterial endotoxin testing (e.g., limulus amebocyte lysate (LAL) testing), dynamic light scattering, liquid chromatography with charged aerosol detector(s), gas chromatography, and/or in vitro translation system (e.g., a rabbit reticulocyte lysate translation system and ³⁵S-methionine).

In some embodiments, a batch of an RNA/LNP drug product formulation (e.g., ones described herein) may be assessed for the quality control parameters (e.g., ones described herein) to determine next action step(s). For example, a batch of an RNA/LNP drug product formulation (e.g., ones described herein) can be designated for one or more further steps of manufacturing and/or distribution if quality assessment indicates that such a batch meets or exceeds the relevant release criteria listed. Otherwise, an alternative action can be taken (e.g., discarding the batch) if such a batch does not meet or exceed the release criteria.

### Example 16: Exemplary Inclusion Criteria

In some embodiments, cancer patients whose tumors express CLDN-18.2 can be selected for treatment with compositions and/or methods described herein. In some embodiments, cancer patients are pancreatic cancer patients. In some embodiments, cancer patients are biliary cancer patients.

In some embodiments, cancer patients who meets one or more of the following disease-specific inclusion criteria are selected for treatment with compositions and/or methods described herein:
1. A CLDN-18.2-positive tumor (regardless of tumor histology) defined as ≥50% of tumor cells with ≥2+ CLDN-18.2 protein staining-intensity as assessed by central testing using a validated immunohistochemistry assay in formalin-fixed, paraffin-embedded (FFPE) neoplastic tissues;
2. Availability of a FFPE tumor tissue sample for CLDN-18.2 testing. New biopsies and archival biosamples are allowed. If archival tissue samples from several points of time are available, the most recent one is preferred;
3. Histological documentation of the original primary tumor via a pathology report.
   a. Histologically confirmed solid tumor that is metastatic or unresectable and for which there is no available standard therapy likely to confer clinical benefit, or the patient is not a candidate for such available therapy; and optionally measurable or evaluable disease per RECIST 1.1; OR
   b. Histologically confirmed unresectable locally advanced or metastatic pancreatic ductal adenocarcinoma without prior palliative chemotherapy; and optionally measurable or evaluable disease per RECIST 1.1; OR
c. Histologically confirmed, unresectable locally advanced or metastatic PDAC without prior palliative chemotherapy eligible for treatment with either nab-paclitaxel+gemcitabine or FOLFIRINOX; and optionally measurable or evaluable disease per RECIST 1.1; OR
d. Histologically confirmed, locally advanced or metastatic BTCs without prior palliative chemotherapy eligible for treatment with cisplatin+gemcitabine; and optionally measurable or evaluable disease per RECIST 1.1.

In some embodiments, cancer patients who meets at least one of the disease-specific inclusive criteria as discussed above and further meets at least one of the following other inclusive criteria are selected for treatment with compositions and/or methods described herein:
1. Be ≥18 years of age.
2. Eastern Cooperative Oncology Group (ECOG) performance status of 0 to 1.
3. Adequate coagulation function at screening as determined by:
   a. International normalized ratio (INR) or prothrombin time ≤1.5× upper limit normal (ULN; unless on therapeutic anticoagulants with values within therapeutic window).
b. Activated partial thromboplastin time (aPTT)≤1.5×ULN (unless on therapeutic anticoagulants with values within therapeutic window).
4. Adequate hematologic function at screening as determined by:
   a. White blood count (WBC)≥3×10⁹/L.
   b. Absolute neutrophil count (ANC)≥1.5×10⁹/L (patient may not use Granulocyte-colony stimulating factor or granulocyte-macrophage colony stimulating factor to achieve these WBC and ANC levels in the past 7 days).
   c. Platelet count ≥100×10⁹/L.
   d. Hemoglobin ≥9.0 g/dL (may not transfuse or use erythropoietin to obtain this level in the past 7 days).
5. Adequate hepatic function at screening as determined by:
   a. Total bilirubin ≤1.5 mg/dL (or ≤2.0 mg/dL for patients with known Gilbert's syndrome or liver metastasis).
   b. Aspartate aminotransferase (AST) and Alanine aminotransferase (ALT)≤2.5×ULN; ≤3 ULN for patients with liver metastasis.
6. Adequate renal function at screening as determined by:
   a. Glomerular filtration rate ≥45 mL/min/1.73 m²—according to the abbreviated Modification of Diet in Renal Disease equation:

GFR=186×(S_{creatinin}^{-1.154})×(age^{-0.203})
(where the serum creatinine level is expressed in mg/dL; multiply it by 0.742 if the patient is female; multiply it by 1.212, if the patient is African-American (Levey et al. 1999).
7. Women of childbearing potential (WOCBP) must have a negative serum (beta-human chorionic gonadotropin) test/value at screening. Patients who are post-menopausal or permanently sterilized can be considered as not having reproductive potential.
8. Women of childbearing potential must agree not to donate eggs (ova, oocytes) for the purposes of assisted reproduction during the treatment regimen until 6 months after the last CLDN-18.2-targeting treatment described herein.
9. Men who are sexually active with WOCBP and who have not had a vasectomy must agree to use a barrier method of birth control, e.g., either condom with spermicidal foam/gel/film/cream/suppository or partner with occlusive cap (diaphragm or cervical/vault caps) with spermicidal foam/gel/film/cream/suppository during the trial and for 6 months after receiving the last dose of a CLDN-18.2-targeting treatment described herein.
10. Men must agree to not donate sperm during the treatment regimen and for 6 months after receiving the last dose of CLDN-18.2-targeting treatment described herein.

### Example 17: Exemplary Exclusion Criteria

In some embodiments, cancer patients whose tumor do not express CLDN-18.2 are not amenable to compositions and/or methods described and/or utilized herein.

In some embodiments, cancer patients who (i) have recently received a cancer treatment; (ii) are concurrently receiving systemic steroid therapy; (iii) have recently had a major surgery; (iv) are suffering from active infection and being treated with an anti-infective therapy; and/or (v) are diagnosed with growing brain or leptomeningeal metastases, are not amenable to compositions and/or methods described and/or utilized herein.

In some embodiments, the following cancer patients may not be recommended for a CLDN-18.2-targeting treatment described herein (e.g., administration of compositions described herein and/or treatment methods described herein).

### Prior and Concomitant Therapy

1. Receiving: radiotherapy, chemotherapy, or molecularly-targeted agents or tyrosine kinase inhibitors within 2 weeks or 5 half-lives (whichever is longer) of the start of a CLDN-18.2-targeting treatment described herein; immunotherapy/monoclonal antibodies within 3 weeks of the start of a CLDN-18.2-targeting treatment described herein; nitrosoureas, antibody-drug conjugates, or radioactive isotopes within 6 weeks of the start of a CLDN-18.2-targeting treatment described herein.
2. Receives concurrent systemic (oral or IV) steroid therapy >10 mg prednisone daily or its equivalent for an underlying condition.
3. Major surgery within the 4 weeks before the first dose of a CLDN-18.2-targeting treatment described herein.
4. Ongoing or active infection requiring IV treatment with anti-infective therapy that has been administered less than 2 weeks prior to the first dose of a CLDN-18.2-targeting treatment described herein.
5. Side effects of any prior therapy or procedures for any medical condition not recovered to National Cancer Institute Common Terminology Criteria for AEs (NCI CTCAE) v.5 Grade ≤1. It should be noted that peripheral neuropathy Grade ≤2 is allowed; alopecia of any grade is allowed.

### Medical Conditions

6. Current evidence of new or growing brain or leptomeningeal metastases during screening. Patients with known brain or leptomeningeal metastases may be eligible if they have:
   a. Radiotherapy, surgery or stereotactic surgery for the brain or leptomeningeal metastases.
   b. No neurological symptoms (excluding Grade ≤2 neuropathy).
   c. Stable brain or leptomeningeal disease on the computer tomography (CT) or magnet resonance imaging (MRI) scan within 4 weeks before signing the informed consent.
   d. Not undergoing acute corticosteroid therapy or steroid taper.
      It should be noted that patients with central nervous system symptoms should undergo a CT scan or MRI of the brain to exclude new or progressive brain metastases. Spinal bone metastases are allowed, unless imminent fracture with cord compression is anticipated.
7. History of seizures other than isolated febrile seizure during childhood; has a history of a cerebrovascular accident or transient ischemic attack less than 6 months before screening.
8. Effusions (pleural, pericardial, or ascites) requiring drainage.
9. History of autoimmune disease active or past including but not limited to inflammatory bowel disease, systemic lupus erythematosus, ankylosing spondylitis, scleroderma, or multiple sclerosis.
10. Active immunologic disorder requiring immunosuppression with steroids or other immunosuppressive agents (e.g., azathioprine, cyclosporine A) with the exception of patients with isolated vitiligo, resolved childhood asthma or atopic dermatitis, controlled hypoadrenalism or hypopituitarism, and euthyroid patients with a history of Grave's disease. Patients with controlled hyperthyroidism must be negative for thyroglobulin, thyroid peroxidase antibodies, and thyroid stimulating immunoglobulin prior to administration of trial treatment.
11. Known history of seropositivity for human immunodeficiency virus with CD4+ T-cell counts <350 cells/μL and with a history of acquired immunodeficiency syndrome-defining opportunistic infections.
12. Known history/positive serology for hepatitis B requiring active antiviral therapy (unless immune due to vaccination or resolved natural infection or unless passive immunization due to immunoglobulin therapy). Patients with positive serology must have hepatitis B viral load below the limit of quantification.
13. Active hepatitis C virus (HCV) infection; patients who have completed curative antiviral treatment with HCV load below the limit of quantification are allowed.
14. Known hypersensitivity to a component of a CLDN-18.2-targeting treatment described herein.
15. Another primary malignancy that has not been in remission for at least 2 years, with the exception of those with a negligible risk of metastasis or death (such as adequately treated carcinoma in situ of the cervix, basal or squamous cell skin cancer, localized prostate cancer, or ductal carcinoma in situ).

### Other Comorbidities

16. Abnormal electrocardiograms that are clinically significant, such as Fridericia-corrected QT prolongation >480 ms.
17. In the opinion of the treating practitioner, has any concurrent conditions that could pose an undue medical hazard or interfere with the interpretation of the treatment results; these conditions include, but are not limited to:
   a. Ongoing or active infection requiring antibiotic/antiviral/antifungal therapy.
   b. Concurrent congestive heart failure (New York Heart Association Functional Classification Class III or IV).
   c. Concurrent unstable angina.
   d. Concurrent cardiac arrhythmia requiring treatment (excluding asymptomatic atrial fibrillation).
   e. Acute coronary syndrome within the previous 6 months.
   f. Significant pulmonary disease (shortness of breath at rest or on mild exertion) for example due concurrent severe obstructive pulmonary disease.
18. Cognitive, psychological or psychosocial impediment that would impair the ability of the patient to receive therapy according to the protocol or adversely affect the ability of the patient to comply with the informed consent process and compliance with the protocol-required visits and procedures.
19. Pregnant or breastfeeding.

### Example 18: Exemplary Dosing Schedule of CLDN-18.2-Targeting Composition Described Herein in Combination with Nab-Paclitaxel and/or Gemcitabine

In some embodiments, pharmaceutical compositions provided herein can be administered to patients with CLDN-18.2 positive cancer in combination with other anti-cancer therapies. In some embodiments, administration involves one or more cycles. In some embodiments, pharmaceutical compositions provided herein can be administered in at least 3-8 cycles.

In some embodiments, a dosing for a CLDN-18.2-targeting composition described herein may be performed at one or more of the levels shown in Table 13 above (see Example 8); in some embodiments, dosing may involve administration of at least one lower dose from Table 13 followed later by administration of at least one higher dose from Table 13.

When given in combination with nab-paclitaxel and gemcitabine, in some embodiments, a CLDN-18.2-targeting composition may be administered before the first infusion of cytotoxic therapy. For example, in some embodiments, a CLDN-18.2-targeting composition may be administered a minimum of 4 hours before the first infusion of cytotoxic therapy (e.g., nab-paclitaxel and gemcitabine). In some embodiments, a CLDN-18.2-targeting composition may be administered at Q3W and chemotherapy will follow the approved schedule according to local guidelines. For example, in some embodiments, a combination treatment comprising a CLDN-18.2-targeting composition and nab-paclitaxel and/or gemcitabine may be administered for at least eight cycles, e.g., in some embodiments according to the schedule as shown in Table 17.

TABLE 17
Exemplary dosing schedule for administration of a CLDN-18.2-targeting
composition and nab-paclitaxel and gemcitabine

| | Cycle | | | | | | | | | | | | | | | | | | | | | | | |
|---|---|---|---|---|---|---|---|---|---|---|---|---|---|---|---|---|---|---|---|---|---|---|---|---|
| | 1 | | | 2 | | | 3 | | | 4 | | | 5 | | | 6 | | | 7 | | | 8 | | |
| | Days | | | | | | | | | | | | | | | | | | | | | | | |
| | 1 | 8 | 15 | 1 | 8 | 15 | 1 | 8 | 15 | 1 | 8 | 15 | 1 | 8 | 15 | 1 | 8 | 15 | 1 | 8 | 15 | 1 | 8 | 15 |
| CLDN-18.2- | X | | | X | | | X | | | X | | | X | | | X | | | X | | | X | | |
| targeting | | | | | | | | | | | | | | | | | | | | | | | | |
| composition | | | | | | | | | | | | | | | | | | | | | | | | |
| Nab- | X | X | X | | X | X | X | | X | X | X | | X | X | X | | X | X | X | | X | X | X | |
| paditaxel | | | | | | | | | | | | | | | | | | | | | | | | |
| Gemcitabine | X | X | X | | X | X | X | | X | X | X | | X | X | X | | X | X | X | | X | X | X | |

As presented in Table 17, the cycle length for CLDN-18.2-targeting treatment is defined as 21 days (q3w) and a CLDN-18.2-targeting composition is given on Day 1 of each cycle. Nab-paclitaxel and gemcitabine is given on Days 1, 8, and 15 every 28 days. Highlighted with bold “x” are shown when Day 1 administration of nab-paclitaxel/gemcitabine matches with administration of an anti-CLDN18.1 composition.

Gemcitabine alone has been used for treatment of pancreatic cancer. For example, a recommended dose of gemcitabine (e.g., Gemzar) is 1000 mg/m² over 30 minutes intravenously. In some embodiments, a recommended treatment schedule is:
Weeks 1-8: weekly dosing for the first 7 weeks followed by 1-week rest.
After Week 8: weekly dosing on Days 1, 8, and 15 of 28-day cycles.

In some embodiments, a CLDN-18.2-targeting composition described herein can be administered in combination with gemcitabine according to the approved dose and treatment schedule of gemicitabine (e.g., Gemzar) as monotherapy for treatment of pancreatic cancer as described above. In some embodiments, a CLDN-18.2-targeting composition described herein can be administered in combination with gemcitabine at a lower dose (e.g., less than 10%, less than 20%, less than 30%, or more) and/or under a less aggressive treatment schedule (e.g., every 10 days, or biweekly, etc.) than the approved dose and treatment schedule for gemicitabine (e.g., Gemzar) as monotherapy for treatment of pancreatic cancer as described above.

Nab-paclitaxel is known to be used in combination with gemcitabine for treatment of metastatic pancreatic adenocarcinoma. For example, a recommended dose of nab-paclitaxel (Abraxane®) is 125 mg/m² administered as an IV infusion over 30-40 minutes on Days 1, 8 and of each 28-day cycle, while gemcitabine should be administered immediately after nab-paclitaxel on Days 1, 8 and 15 of each 28-day cycle.

In some embodiments, a CLDN-18.2-targeting composition described herein can be administered in combination with gemcitabine and nab-paclitaxel according to the approved dose and treatment schedule of nab-paclitaxel/gemcitabine combination treatment as described above. In some embodiments, a CLDN-18.2-targeting composition described herein can be administered in combination with nab-paclitaxel and gemcitabine, at least of which at a lower dose (e.g., less than 10%, less than 20%, less than 30%, or more) and/or under a less aggressive treatment schedule (e.g., every 10 days, or biweekly, etc.) than the approved dose and treatment schedule of nab-paclitaxel/gemcitabine combination treatment as described above.

In some embodiments, pre- and post-medications with antipyretics (e.g., acetaminophen, nonsteroidal anti-inflammatory drugs), anti-emetics, proton-pump inhibitors and anxiolytics per drug/regulatory guidelines may be allowed. In some embodiments, patients should be properly prehydrated before administration of a CLDN-18.2-targeting composition described herein. In some embodiments, corticosteroids should not be used as premedication for a CLDN-18.2-targeting composition described herein.

### Example 19: Exemplary Efficacy Assessments and/or Monitoring

In some embodiments, a cancer patient administered with a CLDN-18.2-targeting composition described herein as a monotherapy or in combination with an additional anti-cancer therapy may be periodically monitored for efficacy of the treatment and/or adjustment of the treatment dosage/schedule.

In some embodiments, efficacy of a treatment may be assessed by computer tomography and/or magnetic resonance imaging scans. In some embodiments, a MM scan may be performed using a 3 Tesla whole body instrument. In some embodiments, when evaluating lesions for efficacy assessments, one or more of following criteria may be used:
Complete response: disappearance of all target lesions. Any pathological lymph nodes (whether target or non-target) must have reduction in short axis to <10 mm.
Partial response: at least a 30% decrease in the sum of diameters of target lesions, taking as reference the baseline sum diameters.
Progressive disease: at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.
Stable disease: neither sufficient shrinkage to qualify for PR nor sufficient increase to qualify for progressive disease, taking as reference the smallest sum diameters while on study.

### REFERENCES

Al-Batran S E, Schuler M R, Zvirbule Z, Manikhas G, Lordick F, Rusyn A, et al. FAST: An international, multicenter, randomized, phase II trial of epirubicin, oxaliplatin, and capecitabine (EOX) with or without IMAB362, a first-in-class CLDN-18.2-targeting antibody, as first-line therapy in patients with advanced CLDN-18.2+ gastric and gastroesophageal junction (GEJ) adenocarcinoma. JCO. 2016; 34(Suppl 18): LBA4001-LBA4001. doi: 10.1200/JCO.2016.34.18_suppl.LBA4001
Al-Batran S E, Zvirbule Z, Lordick F, Thuss-Patience P, Just M, Bitzer M, et al. Phase 1 study of IMAB362 with immunomodulation in patients with advanced gastric cancer. Ann Oncol. 2017a; 28(Suppl 5): 664P. doi: 10.1093/annonc/mdx369.048
Alnylam Pharmaceuticals, I. Patisaran FDA approval (2017).
Bray F, Ferla J M E, Soerjomataram I, Siegel R L, Jemal A, (2018): Global cancer statistics 2018: GLOBOCAN estimates of incidence and mortality worldwide for 36 cancers in 185 countries. First published: 12 Sep. 2018. https://doi.org/10.3322/caac.21492
Cancer Research UK [Accessed Feb. 18, 2020]. Available from www.cancerresearchuk.org/health-professional/cancer-statistics/statistics-by-cancer-type/pancreatic-cancer#heading-Zero)
Cancer.gov Surveillance epidemiology and end results program. 2015 Seer Data. [Accessed Feb. 20, 2019]. Available from: https://seer.cancer.gov/statfacts/html/livibd.html.
Chapman R W. Risk factors for biliary tract carcinogenesis. Ann Oncol. 1999; 10(Suppl 4):S308-S311.
Charbel H, Al-Kawas F H. Cholangiocarcinoma: epidemiology, risk factors, pathogenesis, and diagnosis. Curr Gastroenterol Rep. 2011; 13(2):182-187.
Cidon E U. Resectable cholangiocarcinoma: reviewing the role of adjuvant strategies. Clin Med Insights Oncol. 2016; 10:CMO.S32821.
Coati I, Lotz G, Fanelli G N, Brignola S, Lanza C, Cappellesso R, et al. Claudin-18 expression in oesophagogastric adenocarcinomas: a tissue microarray study of 523 molecularly profiled cases. Br. J Cancer. 2019; 121(3):257-63. doi: 10.1038/s41416-019-0508-4
Coelho T, Adams D, Silva A, Lozeron P, Hawkins P N, Mant T, et al. Safety and efficacy of RNAi therapy for transthyretin amyloidosis. N Engl J Med. 2013; 369(9):819-29. doi: 10.1056/NEJMoa1208760
Conroy T, Desseigne F, Ychou M, Bouche O, Guimbaud R, et al. FOLFIRINOX versus gemcitabine for metastatic pancreatic cancer. N Engl J Med. 2011; 364(19):1817-25.
de Groen P C, Gores G J, Larusso N F, Gunderson L L, Nagorney D M. Biliary tract cancers. N Engl J Med Overseas Ed. 1999; 341(18):1368-1378.
Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45(2):228-47. doi: 10.1016/j.ejca.2008.10.026
England Public Health. National Cancer Intelligence Network: Rare and Less Common Cancers, Incidence and Mortality in England. London: 2015.
Ferlay J, Parkin D M, Steliarova-Foucher E. (2010): Estimates of cancer incidence and mortality in Europe in 2008. In European journal of cancer (Oxford, England: 1990) 46 (4), pp. 765-781. DOI: 10.1016/j.ejca.2009.12.014.
Fitzgerald K, Frank-Kamenetsky M, Shulga-Morskaya S, Liebow A, Bettencourt B R, Sutherland J E, et al. Effect of an RNA interference drug on the synthesis of proprotein convertase subtilisin/kexin type 9 (PCSK9) and the concentration of serum LDL cholesterol in healthy volunteers: a randomised, single-blind, placebo-controlled, phase 1 trial. Lancet. 2014; 383(9911):60-8; doi: 10.1016/S0140-6736(13)61914-5
Gillen S, Schuster T, Meyer Zum Buschenfelde C, Friess H, Kleeff J. Preoperative/neoadjuvant therapy in pancreatic cancer: a systematic review and meta-analysis of response and resection percentages. PLoS Med. 2010; 7(4):e1000267.
Golan T, Raitses-Gurevich M, Kelley R K, et al. Overall survival and clinical characteristics of BRCA-associated cholangiocarcinoma: a multicenter retrospective study. Oncologist. 2017; 22(7): 804-810.
Gourgou-Bourgade S, Bascoul-Mollevi C, Desseigne F, Ychou M, Bouche O, et al. Impact of FOLFIRINOX compared with gemcitabine on quality of life in patients with metastatic pancreatic cancer: results from the PRODIGE 4/ACCORD 11 randomized trial. J Clin Oncol. 2013; 31(1):23-9.
Heinz C, Mitnacht-Kraus R, Kreuzberg M, Wöll S, Sahin U, et al. (2017): 376PPreclinical evaluation of the CLDN-18.2-targeting antibody, IMAB362, in pancreatic carcinoma. In annonc 28 (suppl_5). DOI: 10.1093/annonc/mdx367.010.
Hennedige T P, Neo W T, Venkatesh S K. Imaging of malignancies of the biliary tract—an update. Cancer Imaging. 2014; 14(1):1470-7330.
Holtkamp S, Kreiter S, Selmi A, Simon P, Koslowski M, Huber C, et al. Modification of antigen-encoding RNA increases stability, translational efficacy, and T-cell stimulatory capacity of dendritic cells. Blood 2006; 108(13):4009-17. doi: 10.1182/blood-2006-04-015024
Jemal A, Bray F, Center M M, Ferlay J, Ward E, et al. (2011): Global cancer statistics. In CA: a cancer journal for clinicians 61 (2), pp. 69-90. DOI: 10.3322/caac.20107.
Karanjawala Z E. Illei P B, Ashfaq R, Infante J, Murphy K, et al. (2008): New markers of pancreatic cancer identified through differential gene expression analyses. Claudin 18 and annexin A8. In The American journal of surgical pathology 32 (2), pp. 188-196. DOI:
Khan I, Sarker S J, Hackshaw A. Smaller sample sizes for phase II trials based on exact tests with actual error rates by trading-off their nominal levels of significance and power. Br J Cancer. 2012; 107(11):1801-1809. doi:10.1038/bjc.2012.444
Khan S A, Toledano M B, Taylor-Robinson S D. Epidemiology, risk factors, and pathogenesis of cholangiocarcinoma. HPB. 2008; 10(2):77-82.
Khan Z R, Neugut A I, Ahsan H, Chabot J A. Risk factors for biliary tract cancers. Am J Gastroenterol. 1999; 94(1):149-152.
Kirstein M M, Vogel A. Epidemiology and risk factors of cholangio-carcinoma. Visc Med. 2016; 32(6):395-400.
Kleeff J, Korc M, Apte M, La Vecchia C, Johnson C D et al. Pancreatic Cancer. Nat Rev Dis Primers. 2016; 2:16022.
Lee S S, Kim M H, Lee S K, et al. Clinicopathologic review of 58 patients with biliary papillomatosis. Cancer. 2004; 100(4):783-793.
Lordick F, Schuler M, Al-Batran S E, Zvirbule, Z, Manikhas G, Rusyn A, et al. Claudin 18.2—a novel treatment target in the multicenter, randomized, phase II FAST study, a trial of epirubicin, oxaliplatin, and capecitabine (EOX) with or without the CLDN-18.2-targeting antibody IMAB362 as 1st line therapy in advanced gastric and gastroesophageal junction (GEJ) cancer. Ann Oncol. 2016; 27(suppl 9). doi: 10.1093/annonc/mdw582.001
Manji G A, Olive K P, Saenger Y M, Oberstein P. Current and Emerging Therapies in Metastatic Pancreatic Cancer. Clin Cancer Res. 2017; 23(7):1670-8.
Micke P, Mattsson J S, Edlund K, Lohr M, Jirstrom K, Berglund A, et al. Aberrantly activated claudin 6 and 18.2 as potential therapy targets in non-small-cell lung cancer. Int J Cancer. 2014; 135(9):2206-14. doi: 10.1002/ijc.28857
Mitnacht-Kraus R, Kreuzberg M, Utsch M, Sahin U, Tuereci O. (2017): Preclinical characterization of IMAB362 for the treatment of gastric carcinoma. Poster 378. In Annals of Oncology (28).
Morizane C, Okusaka T, Mizusawa J, Katatyama H, Ueno M, et al. Randomized phase III study of gemcitabine plus S-1 combination therapy versus gemcitabine plus cisplatin combination therapy in advanced biliary tract cancer: a Japan Clinical Oncology Group study (JCOG1113, FUGA-BT) J Clin Oncol. 2018; 36(4_suppl):205.
Morlock R, Krukas-Hampel MR, Türeci Ö. Patient reported outcomes (PRO) results from phase II MONO and FAST zolbetuximab (IMAB362) trials in patients with advanced CLDN-18.2+ gastric and gastroesophageal junction adenocarcinoma (GA/GEJA). Ann Oncol. 2018a; 29(suppl_8). doi: 10.1093/annonc/mdy282.042
Morlock R, Turnbull J, Blahut S, Krukas-Hampel M R, Hawryluk E, et al. Health-related quality-of-life (HRQoL) results from the FAST study: A phase II trial of epirubicin, oxaliplatin, and capecitabine with or without IMAB362 in patients with advanced CLDN-18.2+ gastric and gastroesophageal junction adenocarcinoma. JCO 2018b; 36(4_suppl):54. doi: 10.1200/JCO.2018.36.4_supp1.54
Niimi T, Nagashima K, Ward J M, Minoo P, Zimonjic D B, Popescu N C, et al. claudin-18, a novel downstream target gene for the T/EBP/NKX2.1 homeodomain transcription factor, encodes lung- and stomach-specific isoforms through alternative splicing. Mol Cell Biol. 2001; 21(21):7380-90. doi: 10.1128/MCB.21.21.7380-7390.2001
Orlandini von Niessen A G, Poleganov M A, Rechner C, Plaschke A, Kranz L M, Fesser S, et al. Improving mRNA-Based therapeutic gene delivery by expression-augmenting 3′ UTRs identified by cellular library screening. Mol Ther. 2019; 27(4):824-36. doi: 10.1016/j.ymthe.2018.12.011
Parkin D M, Srivatanakul P, Khlat M. Liver cancer in Thailand I. A case-control study of cholangiocarcinoma. Int J Cancer. 1991; 48(3):323-328.
Primrose J N, Fox R, Palmer D H, Prasad R, Mirza D, et al. Adjuvant capecitabine for biliary tract cancer: The BILCAP randomized study. American Society of Clinical Oncology. 2017; 35:4006-4183.
Quante A S, Ming C, Rottmann M, Engel J, Boeck S, Heinemann V, Westphalen C B, Strauch K. Projections of cancer incidence and cancer-related deaths in Germany by 2020 and 2030. Cancer Med. 2016; 5(9):2649-56.
Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res. 2014; 74(11):2913-21.
Randi G, Malvezzi M, Levi F, Ferlay J, Negri E, et al. Epidemiology of biliary tract cancers: an update. Ann Oncol. 2009; 20(1):146-159.
Rohde C, Yamaguchi R, Mukhina S, Sahin U, Itoh K, et al. (2019): Comparison of Claudin 18.2 expression in primary tumors and lymph node metastases in Japanese patients with gastric adenocarcinoma. In Japanese journal of clinical oncology. DOI: 10.1093/jjco/hyz068.
Sahin U, Koslowski M, Dhaene K, Usener D, Brandenburg G, Seitz G, et al. Claudin-18 splice variant 2 is a pan-cancer target suitable for therapeutic antibody development. Clin Cancer Res. 2008; 14(23):7624-34. doi: 10.1158/1078-0432.CCR-08-1547
Sahin U, Schuler M, Bauer S, Krilova A, Utsch M, Huber C, et al. First-in-human study of IMAB362, an anti-Claudin 18.2 monoclonal antibody, in patients with advanced gastroesophageal cancer. Ann Oncol. 2017; 28(suppl_5). doi: 10.1093/annonc/mdx369.044
Sahin U, Schuler M, Richly H, Bauer S, Krilova A, Dechow T, et al. A phase I dose-escalation study of IMAB362 (Zolbetuximab) in patients with advanced gastric and gastro-oesophageal junction cancer. Eur. J Cancer. 2018; 100:17-26. doi: 10.1016/j.ejca.2018.05.007
Schuler M, Al-Batran S-E, Zvirbule Z, Manikhas G, Lordick F, Rusyn A, et al. Final results of the FAST study, an international, multicenter, randomized, phase II trial of epirubicin, oxaliplatin, and capecitabine (EOX) with or without the CLDN-18.2-targeting antibody IMAB362 as first-line therapy in patients with advanced CLDN-18.2+ gastric and gastroesophageal junction (GEJ) adenocarcinoma. Ann Oncol. 2016; 27(Suppl 6):vi208. doi: 10.1093/annonc/mdw371.06
Seufferlein T, Bachet J B, van Cutsem E, Rougier P. Pancreatic adenocarcinoma. ESMO-ESDO Clinical Practice Guidelines for diagnosis, treatment and follow-up. Ann Oncol. 2012; 23(Suppl 7):vii33-40. doi: 10.1093/annonc/mds224
Shigeyasu K, Tanakaya K, Nagasaka T, Aoki H, Fujiwara T, et al. Early detection of meta-chronous bile duct cancer in Lynch syndrome: report of a case. Surg Today. 2014; 44(10):1975-1981.
Shinozaki A, Shibahara J, Noda N, Tanaka M, Aoki T, Kokudo N, et al. Claudin-18 in biliary neoplasms. Its significance in the classification of intrahepatic cholangiocarcinoma. Virchows Arch. 2011; 459(1):73-80. doi: 10.1007/s00428-011-1092-z
Shroff R T, Borad M J, Xiao L, et al. A phase II trial of gemcitabine (G), cisplatin (C), and nab-paclitaxel (N) in advanced biliary tract cancers (aBTCs) J Clin Oncol. 2017; 35(15_suppl):4018.
Shroff R T, Xiao L, Kaseb A O. A phase II trial of gemcitabine (G), cispla-tin (C), and nab-paclitaxel (N) in advanced biliary tract cancers (aBTCs): Updated survival analysis. J Clin Oncol. 2018; 36(4_suppl):350.
Siegel R, Naishadham D, Jemal A. Cancer statistics, 2013. CA Cancer J Clin. 2013; 63(1):11-30. doi: 10.3322/caac.21166
Siegel R L, Miller K D, Jemal A. Cancer statistics, 2018. CA Cancer J Clin. 2018; 68(1):7-30.
Stadler C R, Bähr-Mahmud H, Celik L, Hebich B, Roth A S, Roth R P. Elimination of large tumors in mice by mRNA-encoded bispecific antibodies. Nat Med. 2017; 23(7):815-7. doi: 10.1038/nm.4356
Tabernero J, Shapiro G I, LoRusso P M, Cervantes A, Schwartz G K, Weiss G J, Paz-Ares L, et al. First-in-humans trial of an RNA interference therapeutic targeting VEGF and KSP in cancer patients with liver involvement. Cancer Discov. 2013; 3(4):406-17. doi: 10.1158/2159-8290. CD-12-0429
Tanaka M, Shibahara J, Fukushima N, Shinozaki A, Umeda M, Ishikawa S, et al. Claudin-18 is an early-stage marker of pancreatic carcinogenesis. In The journal of histochemistry and cytochemistry 2011; 59(10):942-52. doi: 10.1369/0022155411420569
Teague A, Lim K H, Wang-Gillam A. Advanced pancreatic adenocarcinoma: a review of current treatment strategies and developing therapies. Ther Adv Med Oncol. 2015; 7(2):68-84.
Trarbach T, Schuler M, Zvirbule Z, Lordick F, Krilova A, Helbig U, et al. Efficacy and safety of multiple doses of IMAB362 in patients with advanced gastro-esophageal cancer: results of a phase II study. Ann Oncol. 2014; 25(suppl_4)iv218-iv218. doi: 10.1093/annonc/mdu334.21
Türeci O, Koslowski M, Helftenbein G, Castle J, Rohde C, Dhaene K, et al. Claudin-18 gene structure, regulation, and expression is evolutionary conserved in mammals. Gene. 2011; 481(2):83-92. doi: 10.1016/j.gene.2011.04.007
Türeci O, Sahin U, Schulze-Bergkamen H, Zvirbule Z, Lordick F, Koeberle D, et al. A multicentre, phase 2a study of zolbetuximab as a single agent in patients with recurrent or refractory advanced adenocarcinoma of the stomach or lower oesophagus. The MONO study. Ann Oncol. 2019; 30(9):1487-95. doi: 10.1093/annonc/mdz199
Türeci Ö, Mitnacht-Kraus R, Wöll S, Yamada T, Sahin U (2019b): Characterization of zolbetuximab in pancreatic cancer models. In Oncoimmunology 8 (1), pp. e1523096. DOI:
Valle J, Wasan H, Palmer D H, Cunningham D, Anthoney A, et al. Cisplatin plus gemcitabine versus gemcitabine for biliary tract cancer. N Engl J Med. 2010; 362(14):1273-1281.
Von Hoff D D, Ervin T, Arena F P, Chiorean E G, Infante J, Moore M, Seay T, Tjulandin S A, Ma W W, Saleh M N, et al. Increased survival in pancreatic cancer with nab-paclitaxel plus gemcitabine. N Engl J Med. 2013; 369(18):1691-703.
Vos T, Allen C, Arora M, Barber R M, Bhutta Z A, et al. Global, regional, and national incidence, prevalence, and years lived with disability for 310 diseases and injuries, 1990-2015: a systematic analysis for the Global Burden of Disease Study 2015. Lancet. 2016; 388(10053):1545-1602.
Werner J, Combs S E, Springfeld C, Hartwig W, Hackert T, et al. Advanced-stage pancreatic cancer: therapy options. Nat Rev Clin Oncol. 2013; 10(6):323-33.
Wöll S, Schlitter A M, Dhaene K, Roller M, Esposito I, Sahin U, et al. Claudin 18.2 is a target for IMAB362 antibody in pancreatic neoplasms. Int J Cancer. 2014; 134(3):731-9. doi: 10.1002/ijc.28400

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A pharmaceutical composition comprising:
a) at least one single-stranded RNA comprising one or more coding regions that encode an antibody agent that binds preferentially to a Claudin-18.2 (CLDN-18.2) polypeptide relative to a Claudin-18.1 (CLDN-18.1) polypeptide; and
b) lipid nanoparticles;
wherein the at least one single-stranded RNA is encapsulated within at least one of the lipid nanoparticles.

2-4. (canceled)

5. The pharmaceutical composition of any one of claim 1, wherein:
a) the at least one single-stranded RNA encodes both of:
a heavy chain-coding region that encodes at least a variable heavy chain (VH) domain of the antibody agent; and
a light chain-coding region that encodes at least a variable light chain (VL) domain of the antibody agent; or
b) the at least one single-stranded RNA is a first single-stranded RNA comprising a heavy chain-coding region that encodes at least a VH domain of the antibody agent; and
wherein the pharmaceutical composition further comprises a second single stranded RNA comprising a light chain-coding region that encodes at least a VL domain of the antibody agent.

6. (canceled)

7. The pharmaceutical composition of claim 5, wherein:
a) the heavy chain-coding region further encodes a constant heavy chain (CH) domain; and/or
the light chain-coding region further encodes a constant light chain (CL) domain; or
b) the heavy chain-coding region encodes a VH domain, a CHI domain, a CH2 domain, and a C H3 domain of the antibody agent in an immunoglobulin G (IgG) form; and/or
the light chain-coding region encodes a VL domain and a CL domain of the antibody agent in an IgG form.

8. (canceled)

9. The pharmaceutical composition of claim 7, wherein the IgG is IgG 1.

10. The pharmaceutical composition of any one of claim 7,
wherein the heavy chain-coding region consists of or comprises a nucleotide sequence that encodes a full-length heavy chain of Zolbetuximab or Claudiximab; and/or
wherein the light chain-coding region consists of or comprises a nucleotide sequence that encodes a full-length light chain of Zolbetuximab or Claudiximab.

11. (canceled)

12. The pharmaceutical composition of claim 5, wherein the first single-stranded and/or the second single-stranded RNA each independently comprise:
a) a secretion signal encoding region;
b) at least one non-coding sequence element, optionally wherein the at least one non-coding sequence element comprises:
c) a 3′ untranslated region (UTR),
d) a 5′ UTR,
e) a cap structure for co-transcriptional capping of mRNA, and/or
f) a poly adenine (poly A) tail.

13-14. (canceled)

15. The pharmaceutical composition of any one of claim 5, wherein the first single-stranded RNA and/or second single-stranded RNA comprises, in a 5′ to 3′ direction:
a. a 5′ UTR-coding region;
b. a secretion signal-coding region;
c. the heavy chain-coding region;
d. a 3′ UTR-coding region; and
e. a poly A tail-coding region.

16. (canceled)

17. The pharmaceutical composition of claim 15, wherein
a) the poly A tail is or comprises a modified poly A sequence; and/or
b) the first single-stranded and/or the second single-stranded RNA comprises a 5′ cap; and/or
c) the first single-stranded and/or the second single-stranded RNA comprises at least one modified ribonucleotide; and/or
d) the modified ribonucleotide comprises pseudouridine.

18-21. (canceled)

22. The pharmaceutical composition of claim 5, wherein the first single-stranded RNA and the second single-stranded RNA are present in a weight ratio of 3:1 to 1:1, optionally wherein the at least one single-stranded RNA is present at a concentration of 0.5 mg/mL to 1.5 mg/mL.

23. The pharmaceutical composition of any one of claim 5,
wherein the lipid nanoparticles are liver-targeting lipid nanoparticles, optionally wherein the lipid nanoparticles are cationic lipid nanoparticles, optionally wherein the the lipid nanoparticles comprise a (i) polymer-conjugated lipid, (ii) cationic lipid, and (iii) neutral lipid.

24-25. (canceled)

26. The pharmaceutical composition of claim 23, wherein:
a) the polymer-conjugated lipid is present in about 1-2.5 mol % of the total lipids;
b) the cationic lipid is present in 35-65 mol % of the total lipids; and)
c) the neutral lipid is present in 35-65 mol % of the total lipids.

27. The pharmaceutical composition of claim 26, wherein:
the polymer-conjugated lipid is a PEG-conjugated lipid (e.g., 2-[(polyethylene glycol)-2000]-N,N-ditetradecylacetamide),
a) the cationic lipid is ((3-hydroxypropyl)azanediyl)bis(nonane-9, 1-diyl) bis(2-butyloctanoate), and
b) the neutral lipid comprises 1,2-Distearoyl-sn-glycero-3-phosphocholine (DPSC) and/or cholesterol,
wherein the lipid nanoparticles have an average size of about 50-150 nm,
optionally wherein the pharmaceutical composition further comprises:
c) a cryoprotectant; and/or
d) an aqueous buffered solution, optionally wherein the aqueous buffered solution includes sodium ions; and
e) wherein the pharmaceutical composition optionally further comprises a chemotherapeutic agent, optionally wherein the chemotherapeutic agent is a chemotherapeutic agent indicated for treatment of pancreatic cancer.

28-36. (canceled)

37. A method comprising administering a pharmaceutical composition of any one of claim 5 to a subject suffering from a CLDN-18.2-positive solid tumor, optionally wherein the CLDN-18.2-positive tumor is:
a) a pancreatic tumor, a gastric tumor, or a biliary tract tumor; and/or
b) is locally advanced, unresectable, or metastatic,
optionally wherein:
c) the subject has received a pre-treatment sufficient to increase CLDN-18.2 level such that a solid tumor from which the subject is suffering is characterized as a CLDN-18.2-positive solid tumor,
d) the CLDN-18.2-positive tumor is characterized in that ≥50% of tumor cells show ≥2+ CLDN-18.2 protein staining intensity as assessed by an immunohistochemistry assay in formalin-fixed, paraffin-embedded neoplastic tissue from the subject,
d) the pharmaceutical composition is administered as monotherapy,
e) the pharmaceutical composition is administered as part of combination therapy comprising the pharmaceutical composition and a chemotherapeutic agent, and/or
f) the subject has received the chemotherapeutic agent.

38-46. (canceled)

47. The method of claim 37, further comprising:
administering to the subject the chemotherapeutic agent such that the subject is receiving the combination therapy,
optionally wherein:
the chemotherapeutic agent is administered at least four hours after the administration of the pharmaceutical composition,
the chemotherapeutic agent is or comprises gemcitabine and/or paclitaxel for a subject suffering from a CLDN-18.2-positive pancreatic tumor,
the chemotherapeutic agent is or comprises FOLFIRINOX for a subject suffering from a CLDN-18.2-positive pancreatic tumor,
the chemotherapeutic agent is or comprises gemcitabine and/or cisplatin for a subject suffering from a CLDN-18.2-positive biliary tract cancer,
the subject is an adult subject,
the administering is performed by intravenous injection.
the pharmaceutical composition is administered in at least one, at least two, at least three or more dosing cycles,
the pharmaceutical composition is administered as one or more doses per dosing cycle, optionally wherein each dosing cycle is a three-week dosing cycle, and/or
the one or more doses comprise the at least one single-stranded RNA within a range of 0.1 mg/kg to 5 mg/kg body weight of the subject.

48-57. (canceled)

58. In a method of delivering a CLDN-18.2-targeting antibody for cancer treatment in a subject, the improvement comprising administering to the subject the pharmaceutical composition of claim 47.

59. A method of producing a CLDN-18.2-targeting antibody comprising administering to cells the pharmaceutical composition of claim 47 so that the cells express and secrete the CLDN-18.2-targeting antibody encoded by the at least one single-stranded RNA of the pharmaceutical composition, optionally wherein:
the cells are liver cells,
the cells are in a subject,
the CLDN-18.2-targeting antibody is produced at a therapeutically relevant plasma concentration,
the therapeutically relevant plasma concentration is sufficient to mediate cancer cell death through antibody-dependent cellular cytotoxicity (ADCC), and/or
the therapeutically relevant plasma concentration is 0.3-28 μg/mL.

60-64. (canceled)

65. A method comprising a step of:
determining one or more features of an antibody agent expressed from at least one mRNA introduced into cells,
wherein the at least one mRNA comprises one or more of features of at least one or more single-stranded RNA comprising a coding region that encodes an antibody agent that binds preferentially to a Claudin-18.2 (CLDN-18.2) polypeptide relative to a Claudin-18.1 (CLDN-18.1) polypeptide, wherein the one or more features comprises:
(i) protein expression level of the antibody agent;
(ii) binding specificity of the antibody agent to CLDN-18.2;
(iii) efficacy of the antibody agent to mediate target cell death through ADCC; and
(iv) efficacy of the antibody agent to mediate target cell death through complement dependent cytotoxicity (CDC),
optionally wherein the cells are liver cells.

66. A method of characterizing a pharmaceutical composition targeting CLDN-18.2, the method comprising steps of:
contacting cells with at least one pharmaceutical composition as set forth in claim 47; and
detecting the antibody agent produced by the cells,
optionally wherein the method further comprises:
determining one or more features of the antibody agent, wherein the one or more features comprises:
(i) protein expression level of the antibody agent;
(ii) binding specificity of the antibody agent to a CLDN-18.2 polypeptide;
(iii) efficacy of the antibody agent to mediate target cell death through ADCC; and
(iv) efficacy of the antibody agent to mediate target cell death through complement dependent cytotoxicity (CDC),
optionally wherein the cells are liver cells.

67-68. (canceled)

69. The method of claim 65 or 66, wherein the step of determining comprises comparing the one or more features of the antibody agent with that of a reference CLDN-18.2-targeting antibody, optionally wherein:
the step of determining comprises assessing the protein expression level of the antibody agent above a threshold level,
the threshold level is a level that is sufficient to induce ADCC, and/or
the step of determining comprises assessing binding of the antibody agent to a CLDN-18.2 polypeptide, wherein:
the assessing comprises determining binding of the antibody agent to a CLDN-18.2 polypeptide relative to its binding to a CLDN-18.1 polypeptide, or
the assessing comprises determining a binding preference profile of the antibody agent at least comparable to that of a reference CLDN-18.2-targeting antibody, and
wherein the reference CLDN-18.2-targeting antibody is Zolbetuximab or Claudiximab.

70-75. (canceled)

76. The method of claim 69, further comprising:
characterizing the antibody agent as a CLDN-18.2-targeting antibody agent if the antibody agent comprises the following features:
a) protein level of the antibody agent expressed by the cells above a threshold level that is sufficient to induce ADCC;
b) preferential binding of the antibody agent to CLDN-18.2 relative to CLDN-18.1; and
c) killing of at least 50% target cells mediated by ADCC and/or CDC.

77. The method of claim 76, further characterizing the antibody agent as a Zolbetuximab or Claudiximab-equivalent antibody if the features of the antibody are at least comparable to that of Zolbetuximab or Claudiximab.

78. (canceled)

79. The method of any one of claim 65 or 66, wherein the step of determining comprises determining whether, when assessed 48 hours after the contacting, the cells express an antiCLDN-18-2 antibody agent encoded by the at least one single-stranded RNA

80. The method of any one of claim 65, 66 or 76, wherein the step of determining comprises determining one or more of the following features:
a) whether the antibody agent expressed by the cells binds preferentially to a CLDN-18.2 polypeptide relative to a CLDN-18.1 polypeptide;
b) whether the antibody agent expressed by the cells exhibit comparable target specificity to CLDN-18.2 as observed in a flow cytometric binding assay with a reference CLDN-18.2-targeting monoclonal antibody;
c) whether, when assessed 48 hours after incubating immune effector cells (e.g., PBMC cells) and CLDN-18.2 positive cells or CLDN-18.2 negative control cells in the presence of the antibody agent, the CLDN-18.2 positive cells, not the control cells, were lysed;
d) whether the antibody agent expressed by the cells exhibit at least comparable ADCC profile of targeted CLDN-18.2 positive cells as observed with a reference CLDN-18.2-targeting monoclonal antibody in the same concentration; and
e) whether, when assessed 2 hours after incubating CLDN-18.2 positive cells or CLDN-18.2 negative control cells with human serum in the presence of the antibody agent, the CLDN-18.2 positive cells, not the control cells, were lysed.

81. The method of any one of claim 65, 66 or 76, wherein the cells are present in a subject.

82. The method of claim 81, wherein the one or more features include antibody level in one or more tissues in the subject.

83. The method of any one of claim 65, 66 or 76, further comprising: administering the pharmaceutical composition to a group of animal subjects each bearing a human CLDN-18.2 positive xenograft tumor to determine anti-tumor activity if the pharmaceutical composition is characterized as CLDN-18.2-targeting.

84. A method of manufacture, the method comprising steps of:
(A) determining one or more features of a single stranded RNA (ssRNA) or composition thereof, which ssRNA encodes part or all of an antibody agent, which one or more features are selected from the group consisting of:
(i) length and/or sequence of the ssRNA;
(ii) integrity of the ssRNA;
(iii) presence and/or location of one or more chemical moieties of the ssRNA;
(iv) extent of expression of the antibody agent when the ssRNA is introduced into a cell;
(v) stability of the ssRNA or composition thereof;
(vi) level of antibody agent in a biological sample from an organism into which the ssRNA has been introduced;
(vii) binding specificity of the antibody agent expressed from the ssRNA, optionally to CLDN-18.2 and optionally relative to CLDN18.1;
(viii) efficacy of the antibody agent to mediate target cell death through ADCC;
(ix) efficacy of the antibody agent to mediate target cell death through complement dependent cytotoxicity (CDC);
(x) lipid identity and amount/concentration within the composition;
(xi) size of lipid nanoparticles within the composition;
(xii) polydispersity of lipid nanoparticles within the composition;
(xiii) amount/concentration of the ssRNA within the composition;
(xiv) extent of encapsulation of the ssRNA within lipid nanoparticles; and
(xv) combinations thereof;
(B) comparing the one or more features of the ssRNA or composition thereof with that of an appropriate reference standard; and
(C) (i) designating the ssRNA or composition thereof for one or more further steps of manufacturing and/or distribution if the comparison demonstrates that the ssRNA or composition thereof meets or exceeds the reference standard; or
(ii) taking an alternative action if the comparison demonstrates that the ssRNA or composition thereof does not meet or exceed the reference standard.

85. The method of claim 84, wherein the ssRNA is assessed and the one or more further steps of step (C)(i) are or comprise at least formulation of the ssRNA.

86. The method of claim 84 or 85, wherein the composition is assessed and the composition comprises lipid nanoparticles and the one or more further steps of step (C)(i) are or comprise include release and distribution of the composition.

87. The method of claim 85, further comprising administering the formulation to a group of animal subjects each bearing a human CLDN-18.2 positive xenograft tumor to determine anti-tumor activity.

88. A method of determining a dosing regimen of a pharmaceutical composition targeting CLDN-18.2-targeting, the method comprising steps of:
(A) administering a pharmaceutical composition set forth in claim 1 to a
group of animal subjects each bearing a human CLDN-18.2 positive xenograft tumor under a pre-determined dosing regimen;
(B) measuring tumor size of the animal subjects periodically;
(C) (i) increasing the dose and/or dosage frequency if reduction in tumor size after the administration of the pharmaceutical composition is not therapeutically relevant; or
(ii) decreasing the dose and/or dosage frequency if reduction in tumor size after the administration of the pharmaceutical composition is therapeutically relevant, and toxicity effect is shown in at least 30% of the animal subjects; or
(iii) making no changes to the dosage regimen if reduction in tumor size after the administration of the pharmaceutical composition is therapeutically relevant, and no toxicity effect is shown in the animal subjects.
